(19) United States
(12) Patent Application Publication
(10) Pub. No.: US 2022/0333055 A1
(43) Pub. Date: Oct. 20, 2022

(54) EDIBLE MYCELIA AND METHODS OF MAKING THE SAME

(71) Applicant: Ecovative Design LLC, Green Island, NY (US)

(72) Inventors: Jacob Michael Winiski, Troy, NY (US); Jessie Hannah Kaplan-Bie, Somerville, MA (US); Gavin Reim McIntyre, Troy, NY (US); Peter Mueller, Green Island, NY (US); Meghan O'Brien, Halfmoon, NY (US); Alex Carlton, Troy, NY (US); Eben Bayer, Troy, NY (US); Russell Hazen, Clifton Park, NY (US); Stephen Lomnes, Delmar, NY (US); Asa Trench Snyder, Petersburg, NY (US)

(21) Appl. No.: 17/428,946

(22) PCT Filed: Nov. 4, 2020
(86) PCT No.: PCT/US2020/058934

§ 371 Date: Aug. 5, 2021

Related U.S. Application Data
(60) Provisional application No. 62/930,829, filed on Nov. 5, 2019.
(60) Provisional application No. 62/946,752, filed on Dec. 11, 2019.
(60) Provisional application No. 63/028,361, filed on May 21, 2020.
(60) Provisional application No. 63/075,694, filed on Sep. 8, 2020.

(51) Int. Cl. C12M 1/36; C12M 1/12; C12M 1/00; C12M 1/34; A23L 31/00; A01G 18/69

(52) U.S. Cl. CPC C12M 41/48; C12M 25/14; C12M 29/06; C12M 41/12; C12M 41/34; A23L 31/00; A01G 18/69; C12N 1/14

(57) ABSTRACT

An improved mycelium in the form of an edible aerial mycelium that is suitable for use as a food product, including a food ingredient for making mycelium-based food, such as bacon. A method of making an edible aerial mycelium suitable for use as a food product, including a food ingredient. An edible product containing an edible aerial mycelium, and a method of making an edible product comprising an edible aerial mycelium, such as a mycelium-based bacon. A mycelium-based food product having a texture that is analogous to a whole-muscle meat product, wherein that whole-muscle meat product is bacon.

### CROSS-REFERENCE TO RELATED APPLICATIONS

This application claims the benefit of U.S. Provisional Patent Application No. 62/930,829, filed Nov. 5, 2019, entitled TUNABLE MYCOLOGICAL BIOPOLYMER; U.S. Provisional Patent Application No. 62/946,752, filed Dec. 11, 2019, entitled MUSHROOM MYCELIUM AS A MATRIX FOR PRODUCING NON-ANIMAL DERIVED PRODUCTS; U.S. Provisional Patent Application No. 63/028,361, filed May 21, 2020, entitled EDIBLE MYCELIA AND METHODS OF MAKING THE SAME; and U.S. Provisional Patent Application No. 63/075,694, filed Sep. 8, 2020, entitled EDIBLE MYCELIA AND METHODS OF MAKING THE SAME, the disclosures of which are incorporated herein by reference in their entirety.

### INCORPORATION BY REFERENCE TO ANY PRIORITY APPLICATIONS

Any and all applications for which a foreign or domestic priority claims is identified in the Application Data Sheet as filed with the present application are hereby incorporated by reference under 37 C.F.C. § 1.57.

### FIELD

This application relates generally to edible mycelia suitable for use as food products, methods of making edible mycelia and edible mycelial food products, and in particular, to edible aerial mycelia and edible appressed aerial mycelia and methods of making the same.

### BACKGROUND

The projected global population growth to 9.8 billion by 2050 demands an expansion of the scope, diversity, sustainability, and economics of food production (Food and Agriculture Organization of the United Nations, 2019). Over the past two years, the domestic market for alternative protein based products, colloquially “plant-based” foods, has increased dramatically, especially in the refrigerated, plant-based meat category (38% growth over the 104 weeks ending on Dec. 29, 2019 (SPINSscan 2020)). In comparison, conventional meat categories in the United States only grew 0.6% over the 52-week period concluding on Mar. 23, 2019 (IRI 2019). Analysts at the multinational investment bank Barclays estimate the global alternative meat market to reach $140B or 10% of the current meat industry by 2029 (Franck 2019). A bottleneck to achieving this projected growth is an inherent limitation to how plant-based foods are formulated and manufactured.

The vast majority of plant-based foods are categorical alternatives to ground meat products, such as grounds, sausage, burgers, and nuggets. According to retail data compiled weekly by the United States Department of Agriculture's (USDA) Agricultural Marketing Service (AMS), only 36% of beef consumed is ground while the remaining product consists of whole-muscle products such as steak (Agriculture 2020). The fundamental limitation to formulating a whole-muscle meat analog is the present manufacturing processes employed to produce such products from globular protein sources including soy, pea, and wheat.

Since the 1960s, textured soy protein that has undergone High Moisture Extrusion Cooking (HMEC) processes has served as a substitute for minced meat products (Wild 2016). This technology was later applied to Textured Vegetable Proteins (TVP), including wheat and pea, which again have served as grounds. Protein sources derived from fungal mycelium (“mycoprotein”) are inherently grown in fibrils or pellets, but since these cell-lines are cultivated in submerged liquid fermentation the resultant tissue has to be aligned via post processing (Trinci 1992). Shear cell technology, spinning and electrospinning processes have recently been investigated as means to transform dried protein powders into fibrils.

The biological kingdoms of Plantae and Animalia form the core of global agriculture, while in the whole of the kingdom of Fungi, with an estimated 1.5 million species and 117 known edible food species, use of fungal biomass in U.S. food supply is dominated primarily by mushroom production of a single species—Agaricus bisporus (Zarafi, 2019) (USDA National Agricultural Statistics Service (NASS), Agricultural Statistics Board, 2018). The existing production of fungal biomass for use in food supply can be broadly split into two categories: mushroom production, which has been practiced for thousands of years, requires harvest cycles long enough to produce the fruiting body and is limited in final shape and size; and liquid mycoprotein production (introduced in the 1980s as Quorn™) (WIEBE, 2004), which results in a fungal cell “paste” without any fiber alignment, and thus requires further processing to create a desirable cohesive texture (Miri, 2005). By contrast, solid-state mycelium culturing processes can rapidly generate cohesive edible fungal biomass with a structure and texture that may offer unique nutrient profiles as well as sensory and textures suitable for meat alternatives, which may greatly expand the agricultural potential of Fungi.

There remains a need for mycelial food production methods that are more energy and resource efficient, and for novel mycelium-based foods that can serve as meat alternatives and offer unique sensory, nutritive, sustainability, and economic advantages.

### SUMMARY

In a first general aspect, a method of making an edible aerial mycelium is disclosed that includes: providing a growth matrix containing a substrate and a fungal inoculum, wherein the fungal inoculum contains a fungus; incubating the growth matrix as a solid-state culture in a growth environment for an incubation time period; and introducing aqueous mist into the growth environment throughout the incubation time period, or a portion thereof, wherein the aqueous mist has a mist deposition rate and a mean mist deposition rate, and the mean mist deposition rate is less than or equal to about 10 microliter/cm²/hour; thereby producing extra-particle aerial mycelial growth from the growth matrix.

In some aspects, the method of making an edible aerial mycelium can include one or more of the following features. A growth environment can have a growth atmosphere. The growth atmosphere can have a relative humidity, an oxygen (O₂) level and a carbon dioxide (CO₂) level, wherein the CO₂ level can be at least about 0.02% (v/v) and can be less than about 8% (v/v). A mist deposition rate can be less than or equal to about 150 microliter/cm²/hour. The mean mist deposition rate can be less than or equal to about 5 microliter/cm²/hour.

In some aspects, the method of making an edible aerial mycelium can include one or more of the following features. The growth atmosphere can have a CO₂ level within a range of about 0.2% (v/v) to about 7% (v/v). The growth atmosphere can have an O₂ level within a range of about 14% (v/v) to about 21% (v/v). The relative humidity can be at least about 95%, at least about 96% at least about 97%, at least about 98%. The relative humidity can be at least about 99%, or can be about 100%. In some embodiments, the CO₂ level can be at least about 2% (v/v). In other embodiments, the CO₂ level can be less than about 3% (v/v). In some aspects, the incubation time period can be up to about 3 weeks. The incubation time period can be within a range of about 4 days to about 17 days. In some aspects, the method of making an edible aerial mycelium can include one or more of the following features. The method of introducing the aqueous mist into the growth environment can include depositing the aqueous mist onto the growth matrix, the extra-particle aerial mycelial growth, or both. Introducing aqueous mist can include introducing the aqueous mist into the growth environment throughout the entire incubation time period. In other aspects, introducing aqueous mist can include introducing the aqueous mist into the growth environment throughout a portion of the incubation time period, wherein the portion of the incubation time period includes a mycelial vertical expansion phase. The portion of the incubation time period begins during a second day, a third day or a fourth day of the incubation time period. Introducing aqueous mist throughout a portion of the incubation time period can exclude introducing the aqueous mist into the growth environment during a primary myceliation phase.

In some aspects, the method of making an edible aerial mycelium can include one or more of the following features. The mist deposition rate can be less than about 50 microliter/cm²/hour, or can be less than about 25 microliter/cm²/hour. The mist deposition rate can be less than about 10 microliter/cm² hour. In some aspects, the mist deposition rate can be less than about 5 microliter/cm²/hour, less than about 4 microliter/cm²/hour, less than about 3 microliter/cm²/hour, less than about 2 microliter/cm²/hour, or less than about 1 microliter/cm²/hour. In some further aspects, the mean mist deposition rate can be less than or equal to about 3 microliter/cm²/hour. The mist deposition rate can be less than about 2 microliter/cm²/hour, the mean mist deposition rate is less than or equal to about 1 microliter/cm²/hour, or both. The mean mist deposition rate can be at least about 0.01 microliter/cm²/hour. In yet further aspects, the mist deposition rate can be less than about 1 microliter/cm²/hour, the mean mist deposition rate can be less than or equal to about 0.8 microliter/cm²/hour, or both. In some aspects, the mist deposition rate can be at most about 10-fold greater than the mean mist deposition rate, at most about 5-fold greater than the mean mist deposition rate, or at most about 4-fold greater than the mean mist deposition rate. The growth environment can further include an airflow. An airflow can be directed through the growth environment. The airflow can be a substantially horizontal airflow. The substantially horizontal airflow can have a velocity of no greater than about 125 linear feet per minute, no greater than about 110 linear feet per minute, no greater than about 100 linear feet per minute, or no greater than about 90 linear feet per minute.

In some aspects, the method of making an edible aerial mycelium can include one or more of the following features. The aqueous mist can contain one or more solutes. In some aspects, the solute can be an additive. The additive can be an additive as disclosed herein. The aqueous mist can have a conductivity of no greater than about 1,000 microsiemens/cm, no greater than about 800 microsiemens/cm, no greater than about 500 microsiemens/cm, no greater than about 100 microsiemens/cm, or no greater than about 50 microsiemens/cm. The aqueous mist can have a conductivity of no greater than about 25 microsiemens/cm, no greater than about 10 microsiemens/cm, no greater than about 5 microsiemens/cm, or no greater than about 3 microsiemens/cm.

In some aspects, the method of making an edible aerial mycelium can include one or more of the following features. The growth environment can be a dark environment. The growth environment can have a temperature within a range of about 55° F. to about 100° F., or within a range of about 60° F. to about 95° F. The growth environment can have a temperature within a range of about 60° F. to about 75° F., about 65° F. to about 75° F., or about 65° F. to about 70° F.

The method of making an edible aerial mycelium can include one or more of the following features. The fungus can be an edible variety of a filamentous fungus. The fungus can be an edible species of the genus Agrocybe, Albatrellus, Amillaria, Agaricus, Bontdarzewia, Cantharellus, Cerioporus, Climacodon, Cordyceps, Fistulina, Flammulina, Fomes, Fomitopsis, Fusarium, Grifola, Herecium, Hydmem, Hypomyces, Hypsizygus, Ischnoderma, Laetiporus, Laricifomes, Lentinuma, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tyromyvces or Wolfiporia. The fungus can be an edible species of the genus Flammulina, Lentinula, Morchella or Pleurotus. In yet further aspects, the fungus is a species of the genus Pleurotus. The fungus can be Pleurotus citrinopilleatus, Pleurotus columbinus, Pleurotus cornucopiae, Pleurotus dryinus, Pleurotus djamor, Pleurotus eryngii, Pleurotus floridanus, Pleurotus ostreatus, Pleurotus populinus, Pleurotus pulmonarius, Pleurotus sajor-caju or Pleurotus tuber-regium. The fungus can be Pleurotus ostreatus. The method can expressly exclude a fungus of the genus Ganoderma.

In some aspects, the method of making an edible aerial mycelium can include one or more of the following features. The growth matrix can include a nutrient source, wherein the nutrient source is the same or different than the substrate. The growth matrix substrate can be a lignocellulosic substrate.

In some aspects, the method of making an edible aerial mycelium can include one or more of the following features. The method of making an edible aerial mycelium can include removing the extra-particle aerial mycelial growth from the growth matrix, thereby providing an edible aerial mycelium. The edible aerial mycelium does not contain a visible fruiting body. The edible aerial mycelium can be obtained by removing the extra-particle aerial mycelium from the growth matrix as a single contiguous object. The single contiguous object can have a contiguous volume, with a series of linked hyphae over the contiguous volume. The single contiguous object can have a contiguous volume of at least about 15 cubic inches. The single contiguous object can have a contiguous volume of at least about 150 cubic inches, or at least about 300 cubic inches. The edible aerial mycelium can have a mean native thickness of at least about 20 mm, at least about 30 mm, at least about 40 mm or at least about 50 mm. The edible aerial mycelium can have a moisture content of at least about 80% (w/w), at least about 85% (w/w), or can have a moisture content of about 90% (w/w). The edible aerial mycelium can have a mean native density of no greater than about 70 pound per cubic foot (pcf), no greater than about 50 pcf, no greater than about 45 pcf, no greater than about 40 pcf, no greater than about 35 pcf, no greater than about 30 pcf, no greater than about 25 pcf, no greater than about 20 pcf or no greater than about 15 pcf. The edible aerial mycelium can have a mean native density of at least about 1 pcf. The edible aerial mycelium can be suitable for use in the manufacture of a food product. The edible aerial mycelium can be for use in the manufacture of a food product. The food product can be a mycelium-based food product. The mycelium-based food product can be a whole muscle meat alternative. The mycelium-based food product can be a mycelium-based bacon product. The edible aerial mycelium can be a food ingredient. In some aspects, the edible aerial mycelium is not a ground edible aerial mycelium, a minced edible aerial mycelium, or an extruded edible aerial mycelium. In some aspects, the food product is not a ground product, a minced product, or an extruded product.

In a second general aspect, the present disclosure provides: an edible aerial mycelium, wherein the edible aerial mycelium has a grain, and wherein the edible aerial mycelium is characterized as having at least two of the following properties: (i) a mean native density of no greater than about 70 pcf; (ii) a native moisture content of at least about 80% (w/w); (iii) a native Kramer shear force of no greater than about 5 kg/g; (iv) a native ultimate tensile strength of no greater than about 5 psi; (v) a native ultimate tensile strength in a dimension substantially parallel to the grain and a native ultimate tensile strength in a dimension substantially perpendicular to the grain, wherein the native ultimate tensile strength in the dimension substantially parallel to the grain is not more than about 5-fold greater than the native ultimate tensile strength in the dimension substantially perpendicular to the grain; (vi) a native compressive modulus at 10% strain of no greater than about 10 psi; (vii) a native compressive modulus at 10% strain in a dimension substantially parallel to the grain and a native compressive modulus at 10% strain in a dimension substantially perpendicular to the grain, wherein the native compressive modulus at 10% strain in the dimension substantially parallel to the grain is not more than about 20-fold greater than the native compressive modulus at 10% strain in the dimension substantially perpendicular to the grain; (viii) a native compressive stress at 65% strain upon compression in a direction substantially perpendicular to the grain of no greater than about 10 psi; (iv) a mean native thickness of at least about 20 mm; wherein the edible aerial mycelium does not contain a fruiting body.

In some aspects, the edible aerial mycelium can include one or more of the following features. The edible aerial mycelium can be characterized as having at least three of said properties (i) through (ix). The edible aerial mycelium can be characterized as having at least four of said properties (i) through (ix). The edible aerial mycelium can be characterized as having at least five of said properties (i) through (ix). The edible aerial mycelium can be characterized as having at least six of said properties (i) through (ix). The edible aerial mycelium can be characterized as having at least seven of said properties (i) through (ix). The edible aerial mycelium can be characterized as having at least eight of said properties (i) through (ix). The edible aerial mycelium can be characterized as having each and every one of said properties (i) through (ix).

In some aspects, the edible aerial mycelium can include one or more of the following features. The edible aerial mycelium can have a mean native density of at least about 1 pcf. The edible aerial mycelium can have a native moisture content of at least about 85% (w/w). The edible aerial mycelium can have a moisture content of least about 90% (w/w). The edible aerial mycelium can have a native Kramer shear force of no greater than about 3 kg/g. The edible aerial mycelium can have a native ultimate tensile strength of no greater than about 3 psi. The edible aerial mycelium can have a native compressive modulus at 10% strain of no greater than about 5 psi. The edible aerial mycelium can have a native compressive modulus at 10% strain in the dimension substantially parallel to the grain is not more than about 10-fold greater than the native compressive modulus at 10% strain in the dimension substantially perpendicular to the grain.

In some aspects, the edible aerial mycelium can include one or more of the following features. The edible aerial mycelium can have a native compressive modulus at 10% strain in the dimension substantially parallel to the grain is at least about 2-fold greater than the native compressive modulus at 10% strain in the dimension substantially perpendicular to the grain. The edible aerial mycelium can have a native compressive stress at 65% strain upon compression in a direction substantially perpendicular to the grain of no greater than about 1 psi. The edible aerial mycelium can have a native compressive stress at 65% strain upon compression in a direction substantially perpendicular to the grain of no greater than about 0.5 psi. The edible aerial mycelium can have a mean native density of at least about 2 pcf. The edible aerial mycelium can have a mean native density of no greater than about 50 pcf, no greater than about 45 pcf, no greater than about 40 pcf, no greater than about 35 pcf or no greater than about 30 pcf.

In some aspects, the edible aerial mycelium can include one or more of the following features. The edible aerial mycelium can have a mean native density of no greater than about 25 pcf, no greater than about 20 pcf or no greater than about 15 pcf. The edible aerial mycelium can have a mean native density of at least about 2 pcf. The edible aerial mycelium can have a native moisture content of at least about 85% (w/w), or at least about 90% (w/w). The edible aerial mycelium can have a mean native thickness of at least about 30 mm, at least about 40 m or at least about 50 mm. The edible aerial mycelium can have a median native thickness of at least about 30 mm, at least about 40 mm, or at least about 50 mm.

In some aspects, the edible aerial mycelium can include one or more of the following features. The edible aerial mycelium can have a native protein content within a range of about 20% to about 50% (w/w) on a dry weight basis. The edible aerial mycelium can have a native potassium content of at least about 4000 mg per 100 grams of dry aerial mycelium. The edible aerial mycelium can have a native potassium content within a range of about 4000 mg to about 7000 mg potassium per 100 g dry aerial mycelium. The edible aerial mycelium can have a native fat content of at most about 7% (w/w) on a dry weight basis. The edible aerial mycelium can have a native carbohydrate content within a range of about 30% (w/w) to about 60% (w/w) on a dry weight basis. The edible aerial mycelium can have a native inorganic content within a range of about 5% (w/w) to about 20% (w/w) on a dry weight basis. The edible aerial mycelium can have a native dietary fiber content within a range of about 15% (w/w) to about 35% (w/w) on a dry weight basis.

In some aspects, the edible aerial mycelium can include one or more of the following features. The edible aerial mycelium can have an open volume of at least about 50% (v/v), at least about 60% (v/v) or at least about 70% (v/v). The edible aerial mycelium can have a mean hyphal width of at most about 20 microns, at most about 15 microns, or within a range of about 0.2 to about 15 microns.

In some aspects, the edible aerial mycelium can include one or more of the following features. The edible aerial mycelium can be suitable for use in the manufacture of a food product. The edible aerial mycelium can be for use in the manufacture of a food product. The food product can be a mycelium-based food product. The mycelium-based food product can be a whole muscle meat alternative. The mycelium-based food product can be a mycelium-based bacon product. In some further aspects, the edible aerial mycelium is not a ground edible aerial mycelium, a minced edible aerial mycelium, or an extruded edible aerial mycelium. In some aspects, the food product is not a ground product, a minced product, or an extruded product.

In some aspects, the edible aerial mycelium can include one or more of the following features. The edible aerial mycelium can be a growth product of an edible fungus. The edible fungus can be a species of the genus Agrocybe, Albatrellus, Amillaria, Agaricus, Bondarzewia, Cantharellus, Cerioporus, Climacodon, Cordyceps, Fistulina, Flammulina, Fomes, Fomitopsis, Fusarium, Grifola, Herecium, Hythtum, Hypomyces, Hypsizygus, Ischnoderma, Laeuporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia, Trametes, Tuber, Tyromyces or Wolfiporia. The edible fungus can be Pleurotus citrinopilleatus, Pleurotus columbims, Pleurobus cornucopiae, Pleurotus dryinus, Pleurotus djamor, Pleurotus erynghi, Pleurotus floridanus, Pleurotus ostreatus, Pleurotus populinus, Pleurotus pulmonarius, Pleurotus sajor-caju or Pleurotus tuber-regium. The edible fungus can be Pleurotus ostreatus. The edible aerial mycelium can exclude a growth product of a fungus of the genus Ganoderma.

In a third general aspect, the present disclosure provides: an edible product containing an edible aerial mycelium, wherein the edible aerial mycelium is as described above, and wherein the edible product further contains one or more additives.

In some aspects, the edible product containing the edible aerial mycelium can include one or more of the following features. The edible product can include one or more additives, wherein the additive is a fat, a protein, an amino acid, a flavorant, an aromatic agent, a mineral, a vitamin, a micronutrient, a colorant or a preservative; or a combination thereof. The fat can be almond oil, animal fat, avocado oil, butter, canola oil, coconut oil, corn oil, grapeseed oil, hempseed oil, lard, mustard oil, olive oil, palm oil, peanut oil, rice bran oil, safflower oil, soybean oil, sunflower seed oil, vegetable oil, or vegetable shortening; or a combination thereof. The fat can be a plant-based oil or fat. The plant-based oil or fat can be coconut oil or avocado oil. The flavorant can be a smoke flavorant, umami, maple, a salt, a sweetener, a spice, or a meat flavor; or a combination thereof. The smoke flavorant can be applewood flavor, hickory flavor, liquid smoke flavor; or a combination thereof. The salt can be sodium chloride, table salt, flaked salt, sea salt, rock salt, kosher salt or Himalayan salt; or a combination thereof. The sweetener is sugar, cane sugar, brown sugar, honey, molasses, juice, nectar, or syrup; or a combination thereof. The spice can be paprika, pepper, mustard, garlic, chili, jalapeno or capsaicin; or a combination thereof. The colorant can be beet extract, beet juice, or paprika; or a combination thereof. The edible product can contains substantially no amount of an artificial preservative. The edible product can contain substantially no amount of an artificial colorant.

In some aspects, the edible product containing the edible aerial mycelium can include one or more of the following features. The edible product can be a food product. The edible product can be a mycelium-based food product. The mycelium-based food product can be a whole muscle meat alternative. The mycelium-based food product can be a mycelium-based bacon product.

In some aspects, the edible product containing the edible aerial mycelium is not a ground product, a minced product, or an extruded product.

In a fourth general aspect, the present disclosure provides: a batch of edible aerial mycelial panels, wherein each edible aerial mycelial panel in the batch has a grain, and wherein greater than 50% of the panels in the batch is characterized as having at least two of the following properties: (i) a mean native density of no greater than about 70 pounds per cubic foot (pcf); (ii) a native moisture content of at least about 800% (w/w); (iii) a native Kramer shear force of no greater than about 5 kg/g; (iv) a native ultimate tensile strength of no greater than about 5 psi; (v) a native ultimate tensile strength in a dimension substantially parallel to the grain and a native ultimate tensile strength in a dimension substantially perpendicular to the grain, wherein the native ultimate tensile strength in the dimension substantially parallel to the grain is not more than about 5-fold greater than the native ultimate tensile strength in the dimension substantially perpendicular to the grain; (vi) a native compressive modulus at 10%/o strain of no greater than about 10 psi; (vii) a native compressive modulus at 10% strain in a dimension substantially parallel to the grain and a native compressive modulus at 10% strain in a dimension substantially perpendicular to the grain, wherein the native compressive modulus at 10% strain in the dimension substantially parallel to the grain is not more than about 20-fold greater than the native compressive modulus at 10% strain in the dimension substantially perpendicular to the grain; (viii) a native compressive stress at 65% strain upon compression in a direction substantially perpendicular to the grain of no greater than about 10 psi; (ix) a mean native thickness of at least about 20 mm; wherein the edible aerial mycelium does not contain a fruiting body.

In some aspects, the batch of edible aerial mycelial panels can include one or more of the following features. Greater than 50% of the panels in the batch can be characterized as having at least three of said properties (i) through (ix). Greater than 50% of the panels in the batch can be characterized as having at least four of said properties (i) through (ix). Greater than 50% of the panels in the batch can be characterized as having at least five of said properties (i) through (ix). Greater than 50% of the panels in the batch can be characterized as having at least six of said properties (i) through (ix). Greater than 50% of the panels in the batch can be characterized as having at least seven of said properties (i) through (ix). Greater than 50% of the panels in the batch can be characterized as having at least eight of said properties (i) through (ix). Greater than 50% of the panels in the batch can be characterized as having each and every one of said properties (i) through (ix).

In some aspects, the batch of edible aerial mycelial panels can include one or more of the following features. Greater than 50% of the panels in the batch can be suitable for use in the manufacture of a food product. Greater than 50% of the panels in the batch can be for use in the manufacture of a food product. The food product can be a mycelium-based food product. The mycelium-based food product can be a whole muscle meat alternative. The mycelium-based food product can be a mycelium-based bacon product.

In some aspects, a batch of edible aerial mycelial panels can exclude a ground edible aerial mycelium, a minced edible aerial mycelium, or an extruded edible aerial mycelium.

In a fifth general aspect, the present disclosure provides: a method of processing an edible aerial mycelium, including: providing a panel containing an edible aerial mycelium, wherein the edible aerial mycelium is characterized as having a grain; compressing at least a portion of the panel; and cutting at least a portion of the panel in a direction substantially parallel to the grain.

In some aspects, the method of processing an edible aerial mycelium can include one or more of the following features. The edible aerial mycelium can be the edible aerial mycelium as described above. The cutting can include cutting the panel to form at least one panel section. The cutting can include cutting at least one of the panel and the panel section to form at least one strip. The compressing can include compressing at least one of the panel, the at least one panel section, and the at least one strip in a second direction which is substantially non-parallel with respect to the grain. The substantially non-parallel direction can be within a range of 45 degrees to 135 degrees with respect to the grain. The substantially non-parallel direction can be within a range of about 70 degrees to about 110 degrees with respect to the grain. The substantially non-parallel direction can be substantially orthogonal to the grain.

In some aspects, the method of processing an edible aerial mycelium can include one or more of the following features. The compressing can include compressing at least one of the panel, the at least one section and the at least one strip, to about 15% to about 75% of the original panel length or width. The compressing can include compressing at least one of the panel, the at least one section and the at least one strip, to about 30 to about 40% of the original panel length or width. In some aspects, the compressing step can occur before the cutting step. In other aspects, the cutting step can occur before the compressing step.

In some aspects, the method of processing an edible aerial mycelium can include one or more of the following features. The compressing can include compressing the panel to form a compressed panel; and cutting can include cutting the compressed panel to form at least one compressed strip. The cutting can include first cutting the compressed panel to form at least one compressed section; and then cutting the at least one compressed section to form at least one compressed strip.

In some aspects, the method of processing an edible aerial mycelium can include one or more of the following features. The cutting can include first cutting the panel to form at least one strip; and compressing can include compressing the at least one strip to form at least one compressed strip.

In some aspects, the method of processing an edible aerial mycelium can include one or more of the following features. The cutting can include cutting the panel to form at least one section; the compressing can include compressing the at least one section to form at least one compressed section; and the cutting can further include cutting the at least one compressed section to form at least one compressed strip.

In some aspects, the method of processing an edible aerial mycelium can include one or more of the following features. The cutting can include first cutting the panel to form at least one section, then cutting the at least one section to form at least one strip; and compressing the at least one strip.

In some aspects, the method of processing an edible aerial mycelium can include one or more of the following features. The compressing can include applying force to the panel, to the at least one section or to the at least one strip. The compressing can include constraining the panel, the at least one section or the at least one strip during said compression. The compressing can include reducing the volume of each said panel, at least one section or at least one strip by applying the force to the panel, to the at least one section or to the at least one strip. The constraining can include constraining the panel, the at least one section or the at least one strip from movement in a first dimension that is substantially perpendicular to the grain, and further constraining the panel, the at least one section or the at least one strip from movement in a second dimension that is both substantially parallel to the grain and substantially perpendicular to the second direction. The compressing can include applying a force that is less than the force required to shear the panel, the section or the strip.

In some aspects, the method of processing an edible aerial mycelium can include one or more of the following features. The method can include perforating at least one of the panel, the compressed panel, the section, the compressed section, the strip and the compressed strip. The perforating can include needling. The needling can include inserting at least one needle into the outer surface of the panel, the compressed panel, the section, the compressed section, the strip or the compressed strip. The at least one needle can be straight or barbed. The needling can include inserting the at least one needle through an entire thickness of the panel, the compressed panel, the at least one section, the at least one compressed section, the at least one strip or the at least one compressed strip. The at least one strip includes a plurality of strips stacked relative to each other. The perforating can include a first perforation step forming a first perforation pattern, and a second perforation step forming a second perforation pattern. At least one of the density, intensity and shape of the first perforation pattern can be different from the density, intensity and shape of the second perforation pattern. The at least one strip can be a plurality of strips.

In some aspects, the method of processing an edible aerial mycelium can include one or more of the following features. The cutting, the compressing and the perforating can occur simultaneously or stepwise, and when stepwise, according to a variety of sequences. The cutting, the compressing and the perforating can occur simultaneously. The following steps can be performed in the following sequence: the compressing, then the cutting, then the perforating. The following steps can be performed in the following sequence: the compressing, then the perforating, then the cutting. The following steps can be performed in the following sequence: the cutting, then the compressing, then the perforating.

In some aspects, the method of processing an edible aerial mycelium can include one or more of the following features: (a) providing a panel containing an edible aerial mycelium, wherein the edible aerial mycelium is characterized as having a direction of mycelial growth along a first axis; (b) performing a physical method including: compressing the panel in a compressing direction which is substantially non-parallel with respect to the first axis to form a compressed panel; optionally, sectioning the compressed panel to form at least one compressed section; cutting the compressed panel, or optionally the at least one compressed section, in a cutting direction which is substantially parallel to the first axis to form at least one compressed strip; and optionally, perforating the at least one compressed strip to form at least one perforated strip; (c) boiling the at least one compressed strip, or optionally the at least one perforated strip, in a first aqueous saline solution to form at least one boiled strip; (d) brining the at least one boiled strip to provide at least one brined strip; (e) drying the at least one brined strip to provide at least one dried strip; and (f) adding fat to the at least one dried strip to provide at least one fattened strip.

In some aspects, the method of processing an edible aerial mycelium can include one or more of the following features. The aerial mycelial panel can be compressed to about 15% to about 75% of the original panel length or width. The aerial mycelial panel can be compressed to about 30% to about 40% of the original panel length or width. The compressing direction can within a range of greater than 45 degrees and less than 135 degrees, or greater than about 70 degrees and less than about 110 degrees, with respect to the first axis. The compressing direction can be substantially orthogonal to the first axis. The cutting direction can be within a range of plus or minus about 45 degrees with respect to the first axis, or is within a range of plus or minus about 30 degrees with respect to the first axis.

In some aspects, the method of processing an edible aerial mycelium can include one or more of the following features. The method can include sectioning the compressed panel to form at least one compressed section. The sectioning can include cutting the panel in the cutting direction to form the at least one compressed section.

In some aspects, the method of processing an edible aerial mycelium can include one or more of the following features. Compressing the panel, the at least one section or the at least one strip can form a compressed panel, at least one compressed section or at least one compressed strip, respectively. The compressed panel, the at least one compressed section or the at least one compressed strip can be characterized as having a compressive stress at 65% strain of less than about 10 psi. The compressed panel, the at least one compressed section or the at least one compressed strip can be characterized as having a compressive stress at 65% strain of less than about 1 psi. The compressed panel, the at least one compressed section or the at least one compressed strip can be characterized as having a compressive stress at 65% strain of at most about 0.5 psi.

In some aspects, the method of processing an edible aerial mycelium can include one or more of the following features. The boiling the at least one compressed strip, or the at least one perforated strip, can include boiling in first aqueous saline solution having a salt concentration within a range of about 0.1% (w/w) to about 26% (w/w). The first aqueous saline solution can have a salt concentration within a range of about 0.1% to about 15% (w/w). The first aqueous saline solution can have a salt concentration within a range of about 0.5% to about 5% (w/w), or about 1% to about 3%. The first aqueous saline solution further can include at least one an additive. The additive can be an additive as disclosed herein.

In some aspects, the method of processing an edible aerial mycelium can include one or more of the following features. The brining can include treating the at least one boiled strip with a brine fluid to provide the at least one brined strip. The brine fluid can be a second aqueous saline solution having a salt concentration within a range of about 0.1% (w/w) to about 26% (w/w). The second aqueous saline solution can have a salt concentration within a range of about 0.1% to about 15% (w/w). The second aqueous saline solution can have a salt concentration within a range of about 0.5% to about 5% (w/w), or about 1% to about 3%. The brine fluid can further include at least one additive. The additive can be an additive as disclosed herein. The at least one additive is a flavorant, a colorant, or both. The brine fluid can include a smoke flavorant, umami, maple, a salt, a sweetener, a spice, or a combination of any two or more of the foregoing. The method can further include the drying, wherein the drying includes heating the at least one brined strip.

In some aspects, the method of processing an edible aerial mycelium can include one or more of the following features. The method can include fattening the at least one strip to provide at least one fattened strip, wherein the fattening step further includes cooling the at least one fattened strip. The cooling can include cooling the at least one fattened strip until the fat is solidified. Thus, the cooling can set the fat.

In some aspects, the method of processing an edible aerial mycelium can include one or more of the following features. The method can provide at least one finished edible strip. The at least one finished edible strip can beat least one edible mycelium-based bacon strip. The method can further include packaging at least one strip or at least one finished strip. Each at least one strip or at least one finished strip can be a plurality of strips.

In some aspects, the method of processing an edible aerial mycelium can include one or more of the following features. The method can include incorporating at least one additive into at least one of the panel, the at least one section, and the at least one strip. The additive can be an additive as disclosed herein. The at least one additive can be a fat, a protein, an amino acid, a flavorant, an aromatic agent, a mineral, a vitamin, a micronutrient, a colorant or a preservative; or a combination thereof.

In some aspects, the method of processing an edible aerial mycelium can exclude grinding, mincing and/or extruding the edible aerial mycelium.

In a sixth general aspect, the present disclosure provides: an edible strip of mycelium-based bacon, containing: a strip of edible aerial mycelium, wherein the edible aerial mycelium is the edible aerial mycelium as described above, and wherein the strip of edible aerial mycelium contains at least one additive.

In some aspects, the edible strip of mycelium-based bacon can include one or more of the following features. The strip of edible aerial mycelium can be a brined strip. The strip of edible aerial mycelium can be a brined, fatted strip. The strip of edible aerial mycelium can be a boiled, brined and fatted strip. The strip of edible aerial mycelium can be a boiled, brined, compressed and fatted strip. The strip of edible aerial mycelium can be a boiled, brined, compressed, perforated and fatted strip. The strip of edible aerial mycelium can be the at least one finished edible strip described above.

In some aspects, the edible strip of mycelium-based bacon can include one or more of the following features. The strip of edible aerial mycelium can have a moisture content within a range of about 10% to about 90% (w/w). The at least one additive can include a flavorant, a colorant, a fat, or a combination thereof. The at least one additive is coconut oil, sugar, salt, natural flavors and beet juice.

In some aspects, the edible strip of mycelium-based bacon can include one or more of the following features. The edible strip of mycelium-based bacon can be characterized as having a nutritional content including: a fat content within a range of about 5% (w/w) to about 15% (w/w); a total carbohydrate content within a range of about 5% to about 20% (w/w); and a protein content within a range of about 3% to about 15% (w/w). The total carbohydrate content can include about 50% (w/w) dietary fiber. The edible strip of mycelium-based bacon can further contain potassium in an amount within a range of about 0.1% and about 1% (w/w). The edible strip of mycelium-based bacon can further contain sodium in an amount within a range of about 0.5% and about 2% (w/w). The edible strip of mycelium-based bacon can be further characterized as containing substantially no amount of cholesterol.

In some aspects, the edible strip of mycelium-based bacon can include one or more of the following features. The edible strip of mycelium-based bacon can contain sodium in an amount of about 1% (w/w); total carbohydrate in an amount of about 10% to about 15% (w/w); protein in an amount of about 4% to about 7% (w/w); and potassium within a range of about 0.1% to about 0.5% (w/w). The edible aerial mycelium can be Pleurotus mycelium. The edible aerial mycelium can be Pleurotus ostreatus mycelium.

In some aspects, the edible strip of mycelium-based bacon can be characterized as having a length within a range of about 6 to about 10 inches, a width within a range of about 1 to about 2 inches, and a height of no greater than about 0.25 inches.

In some aspects, the edible strip of mycelium-based bacon is not a ground strip of mycelium-based bacon, is not a minced strip of mycelium-based bacon, and is no an extruded strip of mycelium-based bacon.

In a seventh general aspect, the present disclosure provides: a packaged mycelium-based bacon product, containing: a package, containing: at least one edible strip of mycelium-based bacon, as described above; and a label, wherein the label includes nutritional information and cooking instructions for said mycelium-based bacon product. The at least one edible strip of mycelium-based bacon can be a plurality of strips.

In some other general aspects, the present disclosure provides: a method of cooking at least one edible strip of mycelium-based bacon. The method can include one or more of the following features. The method can include at least one of pan frying and baking. The pan frying and baking can be at a temperature within a range of about 275° F. to about 400° F. The cooking can be terminated when the edible strip of mycelium-based bacon is crisp. In some other general aspects, the present disclosure provides: a system for growing an edible aerial mycelium, including: a growth matrix including a substrate and a fungal inoculum, wherein the fungal inoculum contains a fungus; a growth environment configured to incubate the growth matrix as a solid-state culture for an incubation time period; and an atmospheric control system with an electronic controller configured to maintain a carbon dioxide (CO₂) level within the growth environment between at least about 0.02% (v/v) and less than about 8% (v/v) and to introduce aqueous mist into the growth environment throughout the incubation time period, or a portion thereof, at a mist deposition rate of less than or equal to about 150 microliter/cm²/hour, and a mean mist deposition rate over the incubation time period of less than or equal to about 3 microliter/cm²/hour.

In some other general aspects, the present disclosure provides: a method of making an edible appressed mycelium, including: providing a growth matrix containing a substrate and a fungal inoculum, wherein the fungal inoculum contains a fungus; incubating the growth matrix as a solid-state culture in a growth environment for an incubation time period; provided that the growth environment excludes mist; thereby producing extra-particle appressed mycelial growth from the growth matrix.

For purposes of summarizing the invention and the advantages achieved over the prior art, certain objects and advantages of the invention have been described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. In some instances, the drawings may not be drawn to scale.

FIG. 1 illustrates an embodiment of positive gravitropic growth.

FIG. 2 illustrates an embodiment of negative gravitropic growth.

FIG. 3 illustrates an embodiment of horizontal airflow.

FIG. 4 shows an image of an extra-particle aerial mycelium and growth matrix in a Pyrex dish (top, top view; bottom, side view) after removal from a growth chamber, according to Example 4.

FIG. 5 shows an image of an extra-particle aerial mycelium and growth matrix in a Pyrex dish (top, top view; bottom, side view) after removal from a growth chamber, according to Example 5.

FIG. 6 shows an image of an extra-particle aerial mycelium and growth matrix in a Pyrex dish (top, top view; bottom, side view) after removal from a growth chamber, according to Example 6.

FIG. 7 shows an image of an extra-particle aerial mycelium and growth matrix in a Pyrex dish (top, top view; bottom, side view) after removal from a growth chamber, according to Example 7.

FIG. 8 shows an image of extra-particle aerial mycelium prepared according to Example 33 after removal from the growth chamber and prior to extraction from the growth matrix. The inserted ruler shows the thickness of the aerial mycelium and excludes the height of the growth matrix beneath it.

FIG. 9 shows graphs of compressive load (Newtons; N) versus compressive extension (inches) obtained during Kramer shear force testing of aerial mycelia upon shearing in the dimension substantially parallel to the direction of aerial mycelial growth, according to Example 28 (FIG. 9A,B) and Example 32 (FIG. 9C).

FIG. 10 shows graphs of compressive load (Newtons; N) versus compressive extension (inches) obtained during Kramer shear force testing of aerial mycelia upon shearing in the dimension substantially perpendicular to the direction of aerial mycelial growth, according to Example 28.

FIG. 11 shows graphs of compressive load (Newtons; N) versus compressive extension (inches) obtained during Kramer shear force testing of oven dried aerial mycelia upon shearing in the dimension substantially parallel to the direction of aerial mycelial growth, according to Example 28.

FIG. 12 shows a bar graph of protein, fat, ash and carbohydrate content for aerial mycelial panels obtained according to Example 34H.

FIG. 13 illustrates embodiments of processing an aerial mycelial panel, including a cutting step (A) and a compressing step (B).

FIG. 14 illustrates embodiments of perforating an aerial mycelial panel (A) and perforated mycelia with various perforation patterns (B).

### DETAILED DESCRIPTION

US Published Patent Application 2015/0033620, the entire content of which is hereby incorporated by reference in its entirety, describes techniques for growing a material comprising fungal mycelium, referred to as “mycological biopolymer.” As described therein, a mycological biopolymer product provided by the disclosed method is characterized as containing a homogenous biopolymer matrix that is comprised predominantly of fungal chitin and trace residues (e.g., beta-glucan, proteins). The mycological biopolymer is up-cycled from domestic agricultural lignocellulosic waste and is made by inoculating the domestic agricultural lignocellulosic waste substrate with a selected fungus in a container that is sealed off from the ambient environment external to the container. In addition to the substrate and fungal inoculum, the container contains a void space, which space is subsequently filled with a network of undifferentiated fungal mycelium. The biopolymer product grows into the void space of the container, filling the space with an undifferentiated mycelium comprising a chitin-polymer. The chitin-polymer-based mycelium is subsequently extracted from the substrate and dried. As further described in US2015/0033620, the environmental conditions for producing the mycological biopolymer product described therein, i.e. a high carbon dioxide (CO₂) content (about 3% to about 7% by volume) and an elevated temperature (from about 85° F. to about 95° F.), prevent full differentiation of the fungus into a mushroom, as evidenced by the absence of a visible fruiting body.

As described in WO2019/099474A1, the entire contents of which is hereby incorporated by reference in its entirety, another method of growing a biopolymer material employs incubation of a growth media comprised of nutritive substrate inoculated with a fungus in containers that are placed in a closed incubation chamber with air flows passed over each container while the chamber is maintained with predetermined environment of humidity, temperature, carbon dioxide and oxygen.

It is an object of the invention to provide an improved mycelium in the form of an edible aerial mycelium that is suitable for use as a food product, including a food ingredient for making mycelium-based food, such as bacon.

It is another object of the invention to provide a method of making an edible aerial mycelium suitable for use as a food product, including a food ingredient.

It is yet another object of the invention to provide an edible product containing an edible aerial mycelium, and a method of making an edible product comprising an edible aerial mycelium, such as a mycelium-based bacon.

It is another object of the invention to provide a mycelium-based food product having a texture that is analogous to a whole-muscle meat product, wherein that whole-muscle meat product is bacon.

The following discussion presents detailed descriptions of the several embodiments of the present disclosure shown in the Figures. These embodiments are not intended to be limiting, and modifications, variations, combinations, etc., are possible and within the scope of this disclosure.

The present disclosure provides for an aerial mycelium or an appressed mycelium, methods of making an aerial mycelium or an appressed mycelium, and uses thereof.

“Mycelium” as used herein refers to a connective network of fungal hyphae.

“Hyphae” as used herein refers to branched filament vegetative cellular structures that are interwoven to form mycelium.

The aerial and appressed mycelia of the present disclosure are growth products obtained from a growth matrix incubated for a period of time (i.e., an incubation time period) in a growth environment, as disclosed herein.

“Growth matrix” as used herein refers to a matrix containing a fungal-inoculated substrate and an optional nutrition source that is the same or different than the substrate, wherein the substrate, the nutrition source, or both are intended for fungal consumption to support mycelial growth.

In some aspects, a method of making an edible aerial mycelium of the present disclosure comprises placing a growth matrix in contact with a tool. In some aspects, he tool can have a base having a surface area. In some embodiments, the surface area can be at least about 1 square inch. In some embodiments, the surface area can be at most about 2000 square feet. In some embodiments, the growth matrix can be placed in contact with the base, e.g., placed on top of or distributed across the base. In some embodiments, the base can be a planar surface. Non-limiting examples of a tool include a tray, a sheet, a table or a conveyer belt. In some embodiments, the tool can have at least one wall. In some embodiments, the base and the at least one wall can together form a cavity. In some embodiments, the growth matrix can be placed or packed in the tool cavity. In some embodiments, the tool can be an uncovered tool. In some other embodiments, the tool can have a lid, the lid having at least one opening, or the tool can be covered at least in part with a perforated barrier. Non-limiting embodiments of a tool having a lid with an opening are disclosed in US2015/0033620A1. An uncovered tool, or a tool having a lid with an opening or a perforated barrier, and further having growth matrix on or within the tool, can allow for aqueous mist to be deposited onto the growth matrix surface, and/or onto any resulting mycelial growth.

A “native” property as used herein refers to a property associated with a mycelium obtained after an incubation time period has elapsed and upon subsequent removal of the mycelial growth from a growth matrix, and prior to any optional environmental, physical or other post-processing step(s) or excursion(s), whether intentional or unintentional, that substantially alters the property. In some aspects, the present disclosure provides for a mycelium characterized as having one or more “native” properties. In some further aspects, the native property is a native density, a native thickness, a native nutritional content, a native moisture content, a native compressive modulus, and so on. In a nonlimiting example, an environmental step can be a drying step, such as one that reduces the aerial mycelial native moisture content to less than about 80% (w/w), in the case of an aerial mycelium; or less than about 60% (w/w) in the case of an appressed aerial mycelium. In another nonlimiting example, a physical step can be a compression step that substantially reduces the thickness of an aerial mycelial.

“Growth environment” as used herein refers to an environment that supports the growth of mushrooms or mycelia, as would be readily understood by a person of ordinary skill in the art in the mushroom or mycelial cultivation industry, and which contains a growth atmosphere having a gaseous environment of carbon dioxide (CO₂), oxygen (O₂) and a balance of other atmospheric gases including nitrogen (N₂), and is further characterized as having a relative humidity. In some aspects, the growth atmosphere can have a CO₂ level of at least about 0.02% and less than about 8% (v/v). In some other aspects, the growth atmosphere can have an O₂ level of at least about 12% (v/v), or at least about 14% (v/v), and at most about 21% (v/v). In yet other aspects, the growth atmosphere can have an N₂ level of at most about 79% (v/v). Each foregoing CO₂, O₂ or N₂ level is based on a dry gaseous environment, notwithstanding, the growth environment atmosphere having a relative humidity of at least about 90% or at least about 95%.

As disclosed in US2015/0033620, environmental conditions for producing a mycological biopolymer include a CO₂ content of about 3% to about 7% (v/v) to prevent full differentiation of the fungus into a mushroom. Applicant has discovered that an aerial mycelia of the present disclosure can be produced without visible fruiting bodies under conditions wherein aqueous mist is introduced into a growth environment having a growth atmosphere containing much lower CO₂ levels, e.g., levels that approximate ambient earth atmospheric CO₂ levels. Aerial mycelia obtained from a growth environment of circulating mist and an atmosphere having a mean CO₂ level of about 0.04% (v/v) over the incubation time period, or having a mean CO₂ level of about 2% (v/v) over the incubation time period, were similar in yield, thickness, density and morphology to aerial mycelia obtained via growth in an atmosphere having a mean CO₂ content of 5% (v/v) but otherwise identical growth conditions (see Example 36). Applicant has further discovered that an aerial mycelium of the present disclosure can be produced without visible fruiting bodies when grown in the presence of white light (infra).

Thus, in some aspects, a growth atmosphere of the present disclosure can have a CO₂ level of at least about 0.02% (v/v). In some further aspects, the CO₂ level can be at least about 0.04% (v/v). In yet further aspects, the CO₂ level can be within a range of about 0.02% to about 7% (v/v), within a range of about 0.04% to about 7% (v/v), within a range of about 0.1% to about 7% (v/v), within a range of about 0.2% to about 7% (v/v), or within a range of about 1% to about 7% (v/v). In some embodiments, the CO₂ level can be greater than about 2% (v/v). In yet some further embodiments, the CO₂ level can be within a range of about 3% and about 7% (v/v), within a range of about 4% to about 6% (v/v), or within a range of about 5% to about 7% (v/v). In some more particular embodiments, the CO₂ level can be about 1%, about 2%, about 3%, about 4%, about 5%, about 6%, or about 7% (v/v), or any range therebetween. In some embodiments, the CO₂ level is about 5% (v/v).

It is understood that fungal growth requires respiration, which can increase levels of CO₂ and decrease levels of oxygen (O₂) in the growth environment, particularly in an enclosed growth environment such as an incubation chamber or “growth chamber.” In some aspects, the present disclosure provides for a growth environment having a growth atmosphere that is maintained during the incubation time period by replenishing the growth environment with one or more of the atmospheric gases, such as CO₂, replenishing the growth environment with air having the same composition as the target growth atmosphere composition, venting the growth environment to reduce levels of one or more gases, or a combination thereof. In a non-limiting example, if the CO₂ level in a growth chamber is below a target set point, CO₂ gas can be infused into the growth chamber. Conversely, if the CO₂ level exceeds a target set point, then fresh air having the target growth atmosphere composition can be introduced into the growth chamber while venting the chamber to release the existing air having the high CO₂ content. Accordingly, growth chamber atmospheric content can be maintained via CO₂ and fresh air infusion to maintain a target CO₂ set point; as such, O₂ and other atmospheric components are maintained indirectly and fluctuate as a function of fungal respiration.

A growth environment of the present disclosure can be further characterized as having an atmospheric having a pressure as would be readily understood by a person of ordinary skill in the art in the mushroom or mycelial cultivation industry. In a non-limiting embodiment, a growth atmosphere of the present disclosure can have an atmospheric pressure within a range of about 27 to about 31 inches of mercury (Hg), can have an atmospheric pressure of about 29 to about 31 inches Hg, or can have an atmospheric pressure of about 29.9 inches Hg. In some embodiments, a growth environment of the present disclosure can be characterized as having an ambient atmospheric pressure.

“Appressed mycelium” as used herein refers to a continuous mycelium obtained from extra-particle appressed mycelial growth, and which is substantially free of growth matrix.

“Extra-particle appressed mycelial growth” as used herein refers to a distinct mycelial growth that is surface-tracking (thigmotropic), is determinate in growth substantially orthogonal to the surface of a growth matrix, is indeterminate in growth substantially parallel to the surface of the growth matrix, and which exhibits positive gravitropism.

“Determinate growth” as used herein refers to growth that occurs until a maximum final dimension is achieved while growth continues to occur in other dimensions. Either determinate or indeterminate mycelial growth above the surface of a growth matrix defines a mycelium's native thickness.

“Indeterminate growth” as used herein refers to growth that expands indefinitely in a given direction as long as mycelial growth is occurring.

“Positive gravitropism” as used herein refers to growth that preferentially occurs in the direction of gravity.

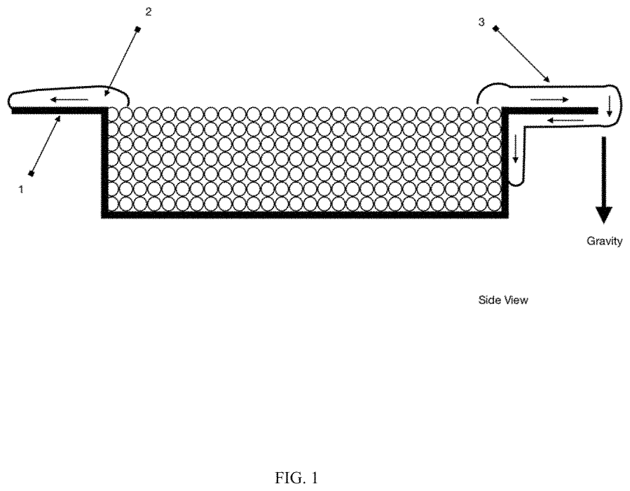

An embodiment of positive gravitropic growth of the present disclosure is illustrated in FIG. 1. Referring to FIG. 1, a growth unit consists of a single tray container with a bottom and side walls, with horizontally oriented rigid surfaces placed as a skirt oriented at the lip of the tray container (1). The tray container contains growth matrix (circles). In the absence of physical water mist deposition, extra-particle mycelial growth (EPM) expands along this horizontal surface as a function of a preference for surface-tracking growth (2). In this case, if/when the expanding EPM reaches the boundary of the horizontally oriented skirt, EPM will default to a combination of surface-tracking and positive gravitropism, continuing to expand along the underside of the skirt or the sidewalls of the tray container (3).

“Aerial mycelium” as used herein refers to mycelium obtained from extra-particle aerial mycelial growth, and which is substantially free of growth matrix.

“Extra-particle aerial mycelial growth” (EPM), as used herein refers to a distinct mycelial growth that occurs upward and outward from the surface of a growth matrix, and which exhibits negative gravitropism.

“Negative gravitropism” as used herein refers to mycelial growth that preferentially occurs in the direction away from gravity.

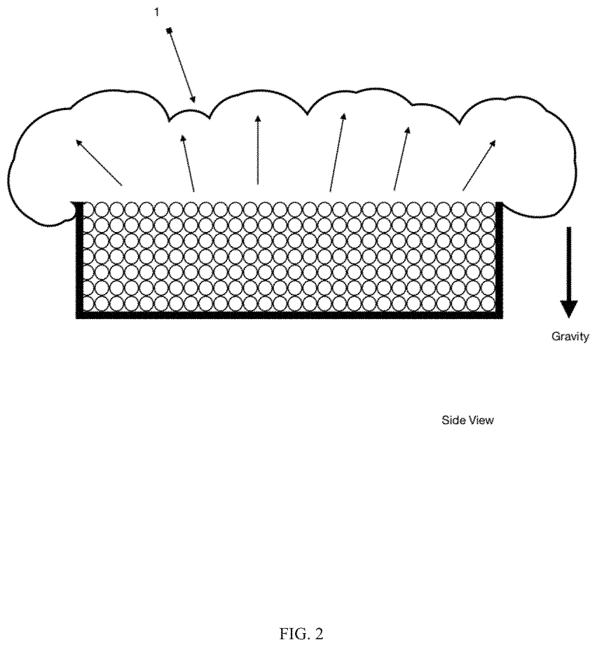

An embodiment of negative gravitropic growth of the present disclosure is illustrated in FIG. 2. Referring to FIG. 2, the growth unit consists of a single tray container with a bottom and side walls. The tray container contains growth matrix (circles). Aqueous mist is deposited directly onto the exposed growth matrix surface, resulting in EPM initiating across the exposed surface. With continued physical aqueous mist deposition, EPM continues to expand forming a contiguous (1), semi-contiguous, or discontiguous volume of extra-particle aerial mycelial growth as a combined function of mist deposition rates and mean mist deposition rates.

As disclosed herein, extra-particle aerial mycelial growth exhibits negatively gravitropism. Without being bound by any particular theory, this may in attributable at least in part to the geometric restriction of the growth format, wherein an uncovered tool having a bottom and side walls contains a growth matrix. With such geometric restriction, growth will primarily occur along the unrestricted dimension(s), which in the scenario is primarily vertically (negatively gravitropic). In a geometrically unrestricted scenario, extra-particle aerial mycelial growth could be described as being neutrally gravitropic, aerial, and radial in which growth will expand in all directions from its point source.

In terms of aerial mycelial growth orientation at a macroscopic scale, the growth orientation is evident as a grain, which is made more evident by the ease with which the tissue tears along this grain, in analogy to the grain of a cut of meat. When looking microscopically, it becomes clear that this grain is a function of aggregations of hyphae that are oriented into larger aligned structures. Hyphal alignments can be measured by methods known in the art (e.g., Boudaoud A. et al., FibrilTool, an ImageJ plug-in to quantify fibrillar structures in raw microscopy images, Nature Protocols, 9, 457-463, 2014, the entire contents of which are hereby incorporated by reference in their entirety), which outputs strengths of hyphal alignment as fractional anisotropy. An aerial mycelium of the present disclosure can have a fractional anisotropy of at least about 5%, or at least about 10%, and can have a fractional anisotropy of at most about 40%.

Thus, an aerial mycelium of the present disclosure can be characterized by its direction of mycelial growth. At a macroscopic scale, the direction of mycelial growth, which may be referred to herein as the “grain,” is generally aligned along a first axis, which may be referred to herein as an “aerial mycelial growth axis.”

In some aspects of the present disclosure, there is provided a method of making an edible aerial mycelium or an edible appressed mycelium. In some aspects, the method comprises: providing a growth matrix comprising a substrate and a fungal inoculum, wherein the fungal inoculum comprises a fungus; and incubating the growth matrix as a solid-state culture in a growth environment for an incubation time period.

Edible mycelia of the present disclosure can be grown in a matter of weeks or days. This feature is of practical value in the production of food ingredient or food product, where time and efficiency are at a premium. Accordingly, the presently disclosed method of making an edible aerial mycelium or an edible appressed mycelium comprises incubating a growth matrix as a solid state culture in a growth environment for an incubation time period of up to about 3 weeks. In some embodiments, the incubation time period can be within a range of about 4 days to about 17 days. In some further embodiments, the incubation time period can be within a range of about 7 days to about 16 days, within a range of about 8 days to about 15 days, within a range of about 9 days to about 15 days, within a range of about 9 days to about 14 days, within a range of about 8 to about 14 days, within a range of about 7 to about 13 days, or within a range of about 7 to about 10 days. In some more particular embodiments, the incubation time period can be about 7 days, about 8 days, about 9 days, about 10 days, about 11 days, about 12 days, about 13 days, about 14 days, about 15 days or about 16 days, or any range therebetween.

In some other embodiments, the incubation time period ends no later than when a visible fruiting body forms; or (ii) the incubation time period ends when a visible fruiting body forms. As disclosed herein, aerial mycelia of the present disclosure can be prepared without the formation of a visible fruiting body, thus, in some embodiments, an incubation time period can end without regard to the formation of a visible fruiting body.

In some further aspects, a method of making an edible aerial mycelium or an edible appressed mycelium of the present disclosure further comprises incubating the growth matrix as a solid-state culture in a growth environment, wherein the growth environment has a temperature that supports mycelial growth. In some embodiments, the growth environment has a temperature within a range of about 55° F. to about 100° F., or within a range of about 60° F. to about 95° F. In some more particular embodiments, the growth environment has a temperature within a range of about 80° F. to about 95° F., or within a range of about 85° F. to about 90° F. throughout the incubation time period. In other embodiments, the growth environment has a temperature within a range of about 60° F. to about 75° F., within a range of about 65° F. to about 75° F., or within a range of about 65° F. to about 70° F. In some embodiments, the growth environment temperature can be tuned to optimize for the growth of a particular fungal genus, species or strain.

In some aspects of the present disclosure, there is provided a method of making an edible aerial mycelium. As disclosed herein, a method of making an edible aerial mycelium of the present disclosure can include introducing aqueous mist into the growth environment. More particularly, the method of making an aerial mycelium can include introducing the aqueous mist into the growth environment throughout the incubation time period. Introducing the aqueous mist into the growth environment can include depositing the aqueous mist onto the growth matrix, the extra-particle aerial mycelial growth that occurs upward and outward from the surface of the growth matrix, or both. More particularly, introducing aqueous mist into the growth environment can include depositing the aqueous mist onto an exposed surface of the growth matrix, an exposed surface of the extra-particle aerial mycelial growth that occurs upward and outward from the surface of the growth matrix, or both.

Further to the discovery of a binary aerial growth response to mist deposition, and that aerial mycelia of the present disclosure can be prepared by introducing mist into the growth environment throughout the incubation time period, Applicant has further discovered that aerial mycelia of the present disclosure can also be prepared by introducing mist into the growth environment throughout a portion of the incubation time period. Applicant has measured vertical expansion kinetics of mycelia over the course of an entire incubation period, and characterized the kinetics as having a primary myceliation phase and a vertical expansion phase (see Example 38). The primary myceliation phase included days 1 to 3 of the incubation time period. Depositing mist throughout a portion of the incubation time period (wherein the portion included the vertical expansion phase), and not depositing mist on days 1 to 3 of the incubation time period, was sufficient to produce aerial mycelium having substantially similar characteristics to aerial mycelia obtained by depositing mist throughout the entire incubation period.

Thus, while some aspects of the present disclosure provide for a method of making an aerial mycelium comprising introducing aqueous mist into the growth environment throughout the incubation time period (i.e., throughout the entire incubation time period), in other aspects, the present disclosure provides for a method of making an aerial mycelium comprising introducing aqueous mist into the growth environment throughout a portion of the incubation time period. In some embodiments, a portion of the incubation time period can comprise a vertical expansion phase. In some further embodiments, a portion of the incubation time period can further comprise at least a portion of a primary myceliation phase. In some other embodiments, a portion of the incubation time period can exclude a primary myceliation phase. In yet some other embodiments, a portion of the incubation time period can consist of a vertical expansion phase. Accordingly, in some aspects, introducing aqueous mist into a growth environment throughout a portion of an incubation time period can comprise introducing the aqueous mist into the growth environment throughout a vertical expansion phase. In some embodiments, introducing aqueous mist into the growth environment throughout a portion of the incubation time period can consist of introducing the aqueous mist into the growth environment throughout a vertical expansion phase, and can exclude introducing aqueous mist during the primary myceliation phase. In some embodiments, the portion of the incubation time period can terminate at the end of a vertical expansion phase, or can terminate at the end of an incubation time period.

In some other aspects, a portion of an incubation time period can begin during a first day, a second day, a third day or a fourth day of the incubation time period. Accordingly, in some aspects, introducing aqueous mist into a growth environment throughout a portion of an incubation time period can comprise introducing aqueous mist into the growth environment during a first, a second, a third or a fourth day of the incubation time period. In some embodiments, the portion of the incubation time period can terminate at the end of a vertical expansion phase, or can terminate at the end of an incubation time period.

In some aspects, the total volume of aqueous mist introduced into the growth environment throughout the incubation period, or a portion thereof, is less than about 200 microliters/cm², is less than about 100 microliters/cm², is less than about 50 microliters/cm², is less than about 25 microliters/cm², is less than about 20 microliters/cm², is less than about 15 microliters/cm², or is less than about 10 microliters/cm². In some further aspects, the total volume of aqueous mist introduced into the growth environment throughout the incubation period, or a portion thereof, is at least about 5 microliters/cm².

In some aspect of the present disclosure, the deposited mist can contain one or more dissolved solutes. Applicant has discovered that aerial mycelial growth can be achieved by depositing aqueous mist containing substantially no amounts of dissolved solute onto the growth matrix and/or the extra-particle aerial mycelial growth produced therefrom. Examples 6 and 7 of the present disclosure each disclose a method of making an aerial mycelium, wherein the deposited mist is sourced from tap water having a conductivity within a range of 400 to 500 microsiemens/cm; Examples 30, 31, 36 and 37 each disclose a method of making an aerial mycelium, wherein the deposited mist is sourced from reverse osmosis filtered water having a conductivity within a range of 20 to 40 microsiemens/cm; and Examples 9 and 10 each disclose a method of making an aerial mycelium, wherein the deposited mist is sourced from distilled water having a conductivity of about 3 microsiemens/cm. Without being bound by any particular theory, Applicant has discovered that the aerial growth response is a binary response to mist deposition, wherein aerial growth does not occur in the absence of mist deposition (a condition that gives rise to appressed mycelia), and wherein aerial growth does occur with mist deposition, even when the mist contains substantially no amounts of dissolved solute. Moreover, the aerial mycelia of the present disclosure have properties including their native thickness that exceed those observed under standard culture conditions, and exceed those of any mycelia found in nature.

Thus, in some aspects, the present disclosure provides for depositing an aqueous mist onto the growth matrix and/or the extra-particle aerial mycelial growth produced therefrom, wherein the aqueous mist can have a conductivity of no greater than about 500 microsiemens/cm. In some further aspects, the aqueous mist conductivity can be no greater than about 400 microsiemens/cm, no greater than about 300 microsiemens/cm, no greater than about 200 microsiemens/cm, or no greater than about 100 microsiemens/cm. In some other aspects, the aqueous mist conductivity can be no greater than about 50 microsiemens/cm, no greater than about 40 microsiemens/cm, no greater than about 30 microsiemens/cm, no greater than about 20 microsiemens/cm, no greater than about 10 microsiemens/cm, or no greater than about 5 microsiemens/cm.

As disclosed herein, in some embodiments, the mist comprises one or more solutes. In some embodiments, the one or more solutes is an additive. Non-limiting examples of additives are disclosed herein.

In some further aspects of the disclosure, the mist that is introduced into the growth environment is characterized as having a mist deposition rate and a mean mist deposition rate.

“Mean mist deposition rate” as used herein refers to a mist deposition rate averaged over an incubation time period. The mean mist deposition rate can be expressed based on a surface area over which the mist is deposited. In a non-limiting example, the mist is deposited on an exposed surface of growth matrix at a mean mist deposition rate of about a microliter per square centimeter of growth matrix per hour. In another non-limiting example, the mist is deposited on an exposed surface of growth matrix containing extra-particle aerial mycelial growth, and the mean mist deposition rate is about 1 microliter per square centimeter of the growth matrix containing the extra-particle aerial mycelial growth per hour. For the purposes of the present disclosure, a mean mist deposition rate of 1 microliter per centimeter squared per hour (1 milliliter/cm/hour) is substantially equivalent to a mean mist deposition rate of 1 milligram per centimeter squared per hour (1 mg/cm²/hour), solute concentration notwithstanding.

In some embodiments, the mean mist deposition rate is less than or equal to about 10 microliter/cm²/hour, is less than or equal to about 5 microliter/cm²/hour, is less than or equal to about 4 microliter/cm²/hour, is less than or equal to about 3 microliter/cm²/hour, or is less than or equal to about 2 microliter/cm/hour. In some embodiments, the mean mist deposition rate is less than or equal to about 1 microliter/cm⁴/hour, is less than or equal to about 0.95 microliter/cm²/hour, is less than or equal to about 0.9 microliter/cm²/hour, less than or equal to about 0.85 microliter/cm²/hour, is less than or equal to about 0.8 microliter/cm²/hour, is less than or equal to about 0.75 microliter/cm²/hour, is less than or equal to about 0.7 microliter/cm²/hour, is less than or equal to about 0.65 microliter/cm²/hour, is less than or equal to about 0.6 microliter/cm²/hour, is less than or equal to about 0.55 microliter/cm²/hour, or is less than or equal to about 0.5 microliter/cm²/hour. In some further embodiments, the mean mist deposition rate is at least about 0.01 microliter/cm²/hour, is at least about 0.02 microliter/cm²/hour, is at least about 0.03 microliter/cm²/hour, is at least about 0.04 microliter/cm/hour or is at least about 0.05 microliter/cm²/hour. In yet some further embodiments, the mean mist deposition rate is within a range of; about 0.01 to about 10 microliter/cm²/hour, about 0.01 to about 5 microliter/cm²/hour, about 0.01 to about 4 microliter/cm²/hour, about 0.01 to about 3 microliter/cm²/hour, about 0.01 to about 2 microliter/cm²/hour, about 0.01 to about 1 microliter/cm²/hour, about 0.01 to about 1 microliter/cm²/hour, about 0.01 to about 0.9 microliter/cm²/hour, about 0.01 to about 0.8 microliter/cm² hour, about 0.01 to about 0.75 microliter/cm²/hour, about 0.01 to about 0.7 microliter/cm²/hour, about 0.02 to about 10 microliter/cm²/hour, about 0.02 to about 5 microliter/cm²/hour, about 0.02 to about 4 microliter/cm²/hour, about 0.02 to about 3 microliter/cm²/hour, about 0.02 to about 2 microliter/cm²/hour, about 0.02 to about 1 microliter/cm²/hour, about 0.02 to about 0.9 microliter/cm²/hour, about 0.02 to about 0.8 microliter/cm² hour, about 0.02 to about 0.75 microliter/cm²/hour, about 0.02 to about 0.7 microliter/cm²/hour, about 0.03 to about 10 microliter/cm²/hour, about 0.03 to about 5 microliter/cm²/hour, about 0.03 to about 4 microliter/cm²/hour, about 0.03 to about 3 microliter/cm²/hour, about 0.03 to about 2 microliter/cm²/hour, about 0.03 to about 1 microliter/cm² hour, about 0.03 to about 0.9 microliter/cm²/hour, about 0.03 to about 0.8 microliter/cm/hour, about 0.03 to about 0.75 microliter/cm²/hour, about 0.03 to about 0.7 microliter/cm²/hour, about 0.04 to about 10 microliter/cm²/hour, about 0.04 to about 5 microliter/cm²/hour, about 0.04 to about 4 microliter/cm²/hour, about 0.04 to about 3 microliter/cm²/hour, about 0.04 to about 2 microliter/cm²/hour, about 0.04 to about 1 microliter/cm²% hour, about 0.04 to about 0.9 microliter/cm²/hour, about 0.04 to about 0.8 microliter/cm²/hour, about 0.04 to about 0.75 microliter/cm²/hour, about 0.04 to about 0.7 microliter/cm²/hour, about 0.05 to about 10 microliter/cm²/hour, about 0.05 to about 5 microliter/cm²/hour, about 0.05 to about 4 microliter/cm²/hour, about 0.05 to about 3 microliter/cm²/hour, about 0.05 to about 2 microliter/cm²/hour, about 0.05 to about 1 microliter/cm²/hour, about 0.05 to about 0.9 microliter/cm²/hour, about 0.05 to about 0.8 microliter/cm²/hour, about 0.05 to about 0.75 microliter/cm²/hour, about 0.05 to about 0.7 microliter/cm²/hour, about 0.1 to about 10 microliter/cm²/hour, about 0.1 to about 5 microliter/cm²/hour, about 0.1 to about 4 microliter/cm %/hour, about 0.1 to about 3 microliter/cm²/hour, about 0.1 to about 2 microliter/cm²/hour, about 0.1 to about 1 microliter/cm² hour, about 0.1 to about 0.9 microliter/cm²/hour, about 0.1 to about 0.8 microliter/cm²/hour, about 0.1 to about 0.75 microliter/cm²/hour, about 0.1 to about 0.7 microliter/cm²/hour, about 0.2 to about 10 microliter/cm²/hour, about 0.2 to about 5 microliter/cm²/hour, about 0.2 to about 4 microliter/cm²/hour, about 0.2 to about 3 microliter/cm²/hour, about 0.2 to about 2 microliter/cm²/hour, about 0.2 to about 1 microliter/cm²/hour, about 0.2 to about 0.9 microliter/cm²/hour, about 0.2 to about 0.8 microliter/cm²/hour, about 0.2 to about 0.75 microliter/cm²/hour, about 0.2 to about 0.7 microliter/cm²/hour, about 0.2 to about 0.6 microliter/cm²/hour, about 0.2 to about 0.5 microliter/cm/hour, about 0.2 to about 0.4 microliter/cm²/hour, about 0.3 to about 0.5 microliter/cm²/hour, about 0.3 to about 0.4 microliter/cm²/hour or about 0.30 to about 0.35 microliter/cm²/hour. In some more particular embodiments, the mean mist deposition rate is about 0.05 microliters/cm²/% our, about 0.10 microliters/cm²/hour, about 0.15 microliters/cm²/hour, about 0.20 microliters/cm²/hour, about 0.25 microliters/cm²/hour, about 0.30 microliters/cm²/hour, about 0.35 microliters/cm²/hour, about 0.40 microliters/cm² hour, about 0.45 microliters/cm² hour, about 0.50 microliters/cm²/hour, about 0.55 microliters/cm²/hour, about 0.60 microliters/cm²/hour, about 0.65 microliters/cm²/hour, about 0.70 microliters/cm²/hour, about 0.75 microliters/cm²/hour, about 0.80 microliters/cm²/hour, about 0.85 microliters/cm²/hour, about 0.90 microliters/cm²/hour, about 0.95 microliters/cm²/hour, or about 1.0 microliters/cm²/hour, or any range therebetween.

In yet some further aspects of the disclosure, the mist that is introduced into the growth environment is characterized as having a mist deposition rate.

“Mist deposition rate” as used herein refers to the rate at which mist is deposited per discrete instance of mist deposition. Thus, “mist deposition rate” may be referred to herein as “instantaneous mist deposition rate” or “momentary mist deposition rate.” The mist deposition rate can be based on or determined by measuring the volume of mist deposited on a surface area over a period of time, wherein the period of time is a fraction of the total incubation time period. In a non-limiting example, the mist is deposited on an exposed surface of growth matrix at a mist deposition rate of about 1 microliter per square centimeter of growth matrix per hour. In another non-limiting example, the mist is deposited on extra-particle aerial mycelial growth, and the mist deposition rate is about 1 microliter per square centimeter of the extra-particle aerial mycelial growth per hour. In some embodiments, the mist deposition rate can be reported as the volume of mist deposited per misting duty cycle. For the purposes of the present disclosure, a mist deposition rate of 1 microliter per centimeter squared per hour (1 milliliter/cm²/hour) is substantially equivalent to a mist deposition rate of 1 milligram per centimeter squared per hour (1 mg/cm²/hour), solute concentration notwithstanding.

In some embodiments, the mist deposition rate is less than about 50 microliter/cm²/hour, is less than about 25 microliter/cm²/hour, is less than about 15 microliter/cm²/hour, is less than about 10 microliter/cm² hour, is less than about 5 microliter/cm²/hour, is less than about 4 microliter/cm²/hour, is less than about 3 microliter/cm²/hour, or is less than about 2 microliter/cm²/hour. In some more particular embodiments, the mist deposition rate is less than about 1 microliter/cm²/hour. In some further embodiments, the mist deposition rate is at least about 0.01 microliter/cm²/hour, is at least about 0.02 microliter/cm²/hour, is at least about 0.03 microliter/cm²/hour, is at least about 0.04 microliter/cm²/hour, or is at least about 0.05 microliter/cm²/hour. In yet some further embodiments, the mist deposition rate is within a range of: about 0.05 to about 0.8 microliter/cm²/hour, about 0.05 to about 0.75 microliter/cm²/hour, about 0.1 to about 0.8 microliter/cm²/hour, about 0.1 to about 0.75 microliter/cm²/hour, about 0.2 to about 0.8 microliter/cm²/hour, about 0.2 to about 0.75 microliter/cm² hour, about 0.2 to about 0.7 microliter/cm²/hour, about 0.2 to about 0.6 microliter/cm²/hour, about 0.2 to about 0.5 microliter/cm²/hour, about 0.2 to about 0.4 microliter/cm/hour, about 0.3 to about 0.5 microliter/cm²/hour, about 0.3 to about 0.4 microliter/cm²/hour or about 0.30 to about 0.35 microliter/cm²/hour. In yet more particular embodiments still, the mist deposition rate is about 0.01 microliters/cm²/hour, about 0.02 microliters/cm²/hour, about 0.03 microliters/cm²/hour, about 0.04 microliters/cm²/hour, about 0.05 microliters/cm²/hour, about 0.10 microliters/cm²/hour, about 0.15 microliters/cm²/hour, about 0.20 microliters/cm²/hour, about 0.25 microliters/cm²/hour, about 0.30 microliters/cm²/hour, about 0.35 microliters/cm² hour, about 0.40 microliters/cm²/hour, about 0.45 microliters/cm²/hour, about 0.50 microliters/cm²/hour, about 0.55 microliters/cm²/hour, about 0.60 microliters/cm²/hour, about 0.65 microliters/cm² hour, about 0.70 microliters/cm² hour, about 0.75 microliters/cm²/hour, about 0.80 microliters/cm²/hour, about 0.85 microliters/cm/hour, about 0.90 microliters/cm²/hour, or about 0.95 microliters/cm²/hour, or any range therebetween.

In some embodiments, the mist deposition rate is at most about 10-fold greater than the mean mist deposition rate. In some further embodiments, the mist deposition rate is at most about 5-fold greater, is at most 4-fold greater, is at most about 3-fold greater, or is at most about 2-fold greater than the mean mist deposition rate. In some embodiments, the mist deposition rate is substantially the same as the mean mist deposition rate. In some more particular embodiments, the mist deposition rate is less than about 2 microliter/cm²/hour and the mean mist deposition rate is less than about 1 microliter/cm²/hour. In yet further embodiments, the mist deposition rate and the mean mist deposition rate are each less than about 1 microliter/cm²/hour. In yet further embodiments still, the mist deposition rate is less than about 1 microliter/cm² hour, and the mean mist deposition rate is less than about 0.5 microliter/cm²/hour.

In other embodiments, the mist deposition rate is at most about about 150 microliter/cm²/hour, is at most about 100 microliter/cm²/hour, is at most about 75 microliter/cm²/hour, is at most about 50 microliter/cm²/hour, or is at most about 25 microliter/cm²/hour. In some further embodiments, the mist deposition rate is at least about 10 microliters/cm²/hour, or is at least about 15 microliters/cm² hour. In some embodiments, the mist deposition rate is at most about 100 microliter/cm² hour, and the mean mist deposition rate is at least about 10 microliters/cm²/hour, or is at least about 15 microliters/cm²/hour.

In some non-limiting embodiments, aqueous mist is introduced into the growth environment via a misting apparatus, which can be incorporated into the growth environment. The apparatus that introduces the aqueous mist can be the same or different than an apparatus that controls relative humidity of the growth environment. Non-limiting examples of a misting apparatus suitable for introducing mist into the growth environment include a high pressure misting pump, a nebulizer, an aerosol generator or aerosolizer, a mist generator, an ultrasonic nebulizer, an ultrasonic aerosol generator or aerosolizer, an ultrasonic mist generator, a dry fog humidifier, an ultrasonic humidifier or an atomizer misting system (including but not limited to a “misting puck”), essentially as described in WO 2019/099474 A1, the entire content of which is hereby incorporated by reference in its entirety, or a print head configured to deposit mist, such as a 3D printer, essentially as described in U.S. patent application Ser. No. 16/688,699, the entire content of which is hereby incorporated by reference in its entirety. In some other non-limiting embodiments, mist can be introduced into the growth environment via modulation of growth environmental factors such as growth environment atmospheric pressure, temperature and/or relative humidity, or via modulation of the growth atmosphere dew point.

In some embodiments, the mist can be continuously introduced into the growth environment. In some further embodiments, the continuous introduction of mist can be pulse width modulated. In some other embodiments, the continuous introduction of mist deposition can occur at a fixed rate. In yet some other embodiments, the continuous introduction of mist deposition can occur at a variable rate.

In other embodiments, the mist can be intermittently introduced into the growth environment. In some further embodiments, the intermittent introduction of mist can occur at a fixed rate. In other further embodiments, the intermittent introduction of mist can occur at a variable rate. In other further embodiments, the intermittent introduction of mist can occur at regular or irregular periods. In other further embodiments, the intermittent introduction of mist can occur with regular or irregular intervals therebetween without mist introduction.

In some embodiments, a misting apparatus can be operated at a particular duty cycle. In some embodiments, the misting apparatus is operated at a duty cycle of about 100%. In some other embodiments, the misting apparatus is operated at a duty cycle of less than 100%. In some embodiments, the misting apparatus is operated at a duty cycle of no greater than about 75%, no greater than about 50%, no greater than about 40%, or no greater than about 30%. In some further embodiments, the misting apparatus is operated at a duty cycle of at least about 1%, at least about 5%, at least about 10%, at least about 15%, at least about 25%. In some more particular embodiments, the misting apparatus is operated within a range of about 5% to about 25%, about 25% to about 50%, about 50% to about 75%, or about 75% to about 100%.

In some embodiments, a duty cycle can be further characterized by a cycle period. Non-limiting examples include a duty cycle period of about 1800 seconds (i.e., about 30 minutes), about 360 seconds, (i.e., about 6 minutes), about 180 seconds (i.e., about 3 minutes), or about 60 seconds (i.e., about 1 minute), or any value or range therebetween. In some embodiments, a duty cycle period can be at most about 60 minutes, at most about 30 minutes, at most about 15 minutes, or at most about 10 minutes. In some other embodiments, a duty cycle period can be at most about 9 minutes, at most about 8 minutes, at most about 7 minutes or at most about 6 minutes.

As disclosed herein, a method of making an aerial mycelium of the present disclosure can include introducing aqueous mist into the growth environment throughout an incubation time period. Introducing aqueous mist “throughout the incubation time period” as used herein refers to introducing the aqueous mist from the beginning of the incubation time period to the end of the incubation time period. In some aspects, introducing aqueous mist into the growth environment can comprise operating a misting apparatus at a duty cycle of greater than zero from the beginning of the incubation time period to the end of the incubation time period. In a non-limiting example, introducing aqueous mist into a growth environment throughout the incubation time period can comprise operating a misting apparatus at a 50% duty cycle from the beginning of the incubation time period to the end of the incubation time period. Further to this non-limiting example, the misting apparatus operating at the 50% duty cycle can have a duty cycle period of at most about 10 minutes. Thus, in this non-limiting example, the misting apparatus can operate (and thus release mist) for 5 minutes out of each 10 minute duty cycle period, and each 10-minute duty cycle period repeats from the beginning of the incubation time period to the end of the incubation time period. Similarly, introducing mist “throughout a portion of the incubation time period” as used herein refers to introducing the mist from the beginning of the portion of the incubation time period to the end of the portion of the incubation time period. In some embodiments, the end of the portion of the incubation time period can be the end of the entire incubation time period. In some aspects, introducing aqueous mist into the growth environment throughout a portion of the incubation time period can comprise operating a misting apparatus at a duty cycle of greater than zero from the beginning of the portion of the incubation time period to the end of the portion of the incubation time period. It will be understood that introducing aqueous mist “throughout the incubation time period” and “throughout a portion of the incubation time period” as used herein can include, but do not require, mist introduction at exactly the beginning, nor exactly the end of the incubation time period or the portion of the incubation time period, for example, in embodiments where the mist is not applied continuously throughout the entirety of the incubation time period or the portion of the incubation time period.

In some aspects, the present disclosure provides for an aqueous mist characterized as having a mean droplet diameter. In some embodiments, the aqueous mist has a droplet diameter within a range of about 1 to about 30 microns, within a range of about 1 to about 25 microns, within a range of about 1 to about 20 microns, within a range of about 1 to about 15 microns, within a range of about 1 to about 10 microns, or within a range of about 5 to about 10 microns.

The present disclosure provides for a growth environment atmosphere characterized as having a relative humidity sufficient to support mycelial growth. In some aspects, the growth environment of the present disclosure can have a relative humidity of at least about 95%. In some more particular aspects, the relative humidity can be at least about 96%, or is at least about 97%. In some even more particular aspects, the relative humidity can be at least about 98%, is at least about 99%, or is about 100%. In some embodiments, the relative humidity can be 99.0%, 99.1%, 99.2%, 99.3%, 99.4%, 99.5%, 99.6%, 99.7%, 99.8% or 99.9%; or any range therebetween. Means of introducing and regulating relative humidity of a growth environment suitable for the growth of mushrooms and/or mycelia would be readily understood by a person of ordinary skill in the art in the mushroom or mycelial cultivation industry. The relative humidity can be controlled independent of misting using conventional heating, ventilation and air conditioning (HVAC) practices. As disclosed herein, methods of making aerial mycelia of the present disclosure can include introducing aqueous mist into the growth environment. Accordingly, the introduction of mist can be increased if the growth environment relative humidity drops below a target value, or can be decreased if the growth environment relative humidity increases beyond a target value.

In some aspects of the present disclosure, the growth environment suitable for the growth of the aerial or appressed mycelia of the present disclosure can be a dark environment.

“Dark environment” as used herein in connection with a growth environment would be readily understood by a person of ordinary skill in the art in the mushroom or mycelial cultivation industry, and refers to an environment without natural or ambient light, and without growing lights.

Exposing fungi to white light, and especially blue light, has been associated with the induction of fruiting and the enhancement of production efficiency of oyster mushrooms (e.g., see Roshita & Goh, AIP Conference Proceedings 2030, 020110 (2018)), the entire contents of which are hereby incorporated by reference in their entirety). Surprisingly, an aerial mycelium with no visible fruiting bodies can be prepared by the methods of the present disclosure in the presence of white light, which includes blue light. Aerial mycelium prepared in the presence of white light was consistent in yield, thickness, density, morphology and in the absence of visible fruiting bodies when compared to control aerial mycelia produced under the same growth conditions but in a dark environment (e.g., see Example 37).

In some aspects of the present disclosure, the growth environment suitable for the growth of the aerial or appressed mycelia of the present disclosure is characterized as having an airflow. In some further aspects, the air composition of the airflow can be substantially the same as the composition of the growth environment atmosphere.

“Horizontal air flow” as used herein refers to flows of air directed substantially parallel to the surface of a growth matrix and any subsequent extra-particle mycelial growth.

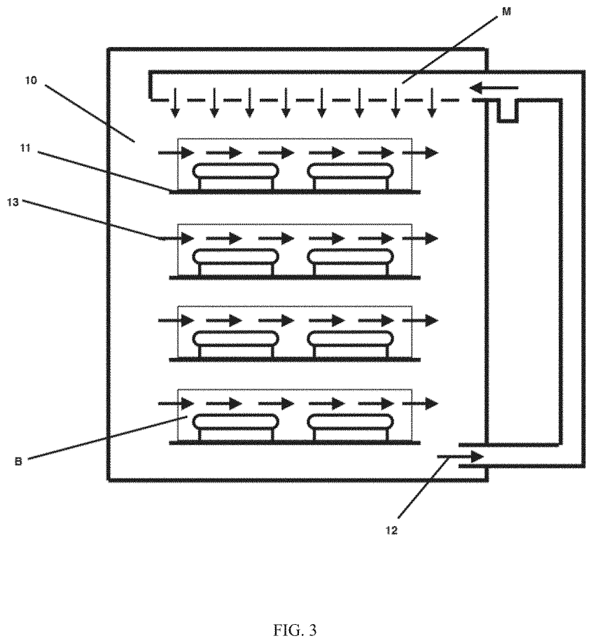

An embodiment of horizontal airflow of the present disclosure is illustrated in FIG. 3. Referring to FIG. 3, the method of growing a mycelium of the present disclosure employs a closed incubation chamber 10 having a plurality of vertically spaced apart shelves 11 and transparent front walls (not shown) for viewing the interior of the chamber 10. In addition, an air flow system 12 is connected with the chamber 10 for directing substantially horizontal air flows across the chamber 10 as indicated by the arrows 13 from one side of the chamber 10 to and through the opposite side of the chamber 10. As illustrated, the air flow system 12 includes a manifold M in the upper part of the chamber 10 for distributing humidified air across the top of the chamber 10 for cascading down the shelves 11 until being recirculated on the bottom right for re-humidification. Each shelf 11 of the chamber 10 is sized to receive an air box B that contains two containers 14 each of which contains a growth media 15 comprised of nutritive substrate and a fungus.

Thus, in some other aspects the method of preparing an aerial or appressed mycelium of the present disclosure can include directing an airflow through the growth environment. In some embodiments, the airflow is a substantially horizontal airflow. In some embodiments, the substantially linear air flow can be have a velocity of no greater than about 350 linear feet per minute (lfm), or a velocity no greater than about 300 lfm. In other embodiments, the substantially horizontal airflow can have a velocity of no greater than about 275 lfm, a velocity of no greater than about 175 lfm, a velocity of no greater than about 150 lfm, a velocity of no greater than about 125 lfm, or a velocity of no greater than about 110 lfm. In some further embodiments, the velocity is at least about 5 lfm, at least about 10 lfm, at least about 15 lfm, at least about 20 lfm, at least about 25 lfm, at least about 30 lfm, at least about 35 lfm, at least about 40 lfm, at least about 45 lfm or at least about 50 lfm. In some more particular embodiments, the substantially horizontal airflow has mean velocity of about 5 lfm, about 10 lfm, about 15 lfm, about 20 lfm, about 25 lfm, about 30 lfm, about 35 lfm, about 40 lfm, about 45 lfm, about 50 lfm, about 55 lfm, about 60 lfm, about 65 lfm, about 70 lfm, about 75 lfm, about 80 lfm, about 85 lfm, about 90 lfm, about 95 lfm, about 100 lfm, about 105 lfm, about 110 lfm, about 115 lfm or about 120 lfm. In some more particular embodiments still, the substantially horizontal air flow can have a velocity within a range of about 5 lfm to about 125 lfm. In yet more particular embodiments, the substantially horizontal air flow can have a velocity within a range of about 5 lfm and about 40 lfm. In other embodiments, the substantially horizontal air flow can have a velocity within a range of about 40 lfm to about 120 lfm. Without being bound to any particular theory, the flows of air can facilitate the distribution of mist throughout the growth environment, can facilitate the distribution of mist onto the growth matrix surface and/or extra-particle mycelial growth, or both. The air flow and misting apparatus can be tuned in concert to achieve the desired mist deposition rate and/or mean mist deposition rate, and to tune the mycelial tissue morphology.

In another aspect, the present disclosure provides an aerial mycelium. In a further aspect, the aerial mycelium does not contain a visible fruiting body.

In another aspect, the present disclosure provides an appressed mycelium. In a further aspect, the aerial mycelium does not contain a visible fruiting body.

“Fruiting body” as used herein refers to a stipe, pileus, gill, pore structure, or a combination thereof.

In a further aspect, the present disclosure provides for an aerial or an appressed mycelium characterized as having particular physicochemical properties.

In some embodiments, a mycelium of the present disclosure is characterized as having a native moisture content. In some embodiments, the native moisture content is expressed as a mean native moisture content.

“Native moisture content” as used herein refers to the moisture content of a mycelium obtained after an incubation time period has elapsed and the resulting mycelial growth has been removed from a growth matrix, and prior to performing any optional environmental, physical or other post-processing step(s) that may increase or decrease the moisture content of the mycelium so obtained.

In some embodiments, an aerial mycelium of the present disclosure can have a native moisture content of greater than about 80% (w/w). In some further embodiments, an aerial mycelium of the present disclosure can have a native moisture content of at least about 85% (w/w), or at least about 90% (w/w). In some embodiments, an aerial mycelium of the present disclosure can have a native moisture content of at most about 95% (w/w). In some more particular embodiments, an aerial mycelium can have a native moisture content of about 81% (w/w), about 82% (w/w), about 83% (w/w), about 84% (w/w), about 85% (w/w), about 86% (w/w), about 87% (w/w), about 88% (w/w), about 89% (w/w), about 90%/a (w/w), about 91% (w/w), about 92% (w/w), about 93% (w/w), about 94% (w/w) or about 95% (w/w), or any range therebetween. Typically, an aerial mycelium of the present disclosure has a native moisture content of about 90% (w/w).

In some embodiments, an appressed mycelium of the present disclosure has a native moisture content of not more than about 80% (w/w), for example, within a range of about 70% (w/w) to about 80% (w/w).

In some embodiments, a mycelium of the present disclosure is characterized as having a native thickness. In some embodiments, the native thickness is expressed as a mean native thickness as determined from sampling over the volume of the mycelium. Typically, the native mycelial thickness is determined from a mycelium obtained after an incubation time period has elapsed and the resulting extra-particle mycelial growth has been removed from a growth matrix, and prior to performing any optional environmental, physical or other post-processing step(s) that may compress or expand the thickness of the mycelium so obtained.

In some aspects, an aerial mycelium of the present disclosure has a native thickness of greater than about 10 mm. In some embodiments, an aerial mycelium of the present disclosure has a native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the native thickness is a mean native thickness. Thus, in some further embodiments, an aerial mycelium of the present disclosure has a mean native thickness of at least about 15 mm, at least about 20 mm, at least about 25 mm, at least about 30 mm, at least about 35 mm, at least about 40 mm, at least about 45 mm, at least about 50 mm, at least about 55 mm or at least about 60 mm. In some embodiments, the native thickness is a median native thickness. Thus, in some further embodiments, an aerial mycelium of the present disclosure has a median native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, or at least about 65 mm. In some embodiments, the native thickness is a maximum native thickness. Thus, in some further embodiments, an aerial mycelium of the present disclosure has a maximum native thickness of at most about 150 mm, at most about 125 mm, at most about 100 mm, at most about 95 mm, at most about 90 mm, or at most about 85 mm.

In some other aspects, at least a portion of an aerial mycelium (or an aerial mycelial panel) of the present disclosure has a native thickness of greater than about 10 mm. In some embodiments, at least a portion of an aerial mycelium of the present disclosure has a native thickness of at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm or at least about 80 mm. In some more particular embodiments, the portion is at least about 10% at least about 20%, at least about 30%), at least about 40%, at least about 50%, at least about 60% at least about 70%, at least about 80% or at least about 90% of the aerial mycelium.

Thus, in some embodiments, the present disclosure provides for an aerial mycelium, wherein at least 25% of the aerial mycelium (i.e., at least 25% of a single aerial mycelial panel) can have a native thickness of at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the present disclosure provides for an aerial mycelium, wherein at least 50% of the aerial mycelium can have a native thickness of at least about 20 mm, at least about 25 mm, at least about 30 mm, at least about 35 mm, at least about 40 mm, at least about 45 mm, at least about 50 mm, at least about 55 mm, at least about 60 mm, at least about 65 mm or at least about 70 mm. In some embodiments, the present disclosure provides for an aerial mycelium, wherein at least 75% of the aerial mycelium can have a native thickness of at least about 20 mm, at least about 25 mm, at least about 30 mm, at least about 35 mm, at least about 40 mm, at least about 45 mm, at least about 50 mm, at least about 55 mm, or at least about 60 mm. In a nonlimiting example, there is provided an aerial mycelium, wherein 75% of the aerial mycelium has a thickness of about 54 mm, 50% of the aerial mycelium has a thickness of about 66 mm, and 25% of the aerial mycelium has a thickness of about 70 mm (e.g., see Example 39, Table 1, Panel A).

In some further embodiments, an aerial mycelium of the present disclosure can have a native thickness of at least about 20 mm, at least about 30 mm or at least about 40 mm over at least 60% of the aerial mycelium. In yet further embodiments, an aerial mycelium of the present disclosure can have a native thickness of at least about 20 mm, at least about 30 mm or at least about 40 mm over at least 70% of the aerial mycelium. In even more particular embodiments, an aerial mycelium of the present disclosure can have a native thickness of at least about 20 mm or at least about 30 mm over at least 70%/o of the aerial mycelium. In some more particular embodiments still, an aerial mycelium of the present disclosure can have a native thickness of at least about 20 mm over at least 80% of the aerial mycelium. In some preferred embodiments, an aerial mycelium of the present disclosure can have a native thickness of at least about 20 mm over at least 90% of the aerial mycelium.

In some aspects, a mycelium of the present disclosure is characterized as having a surface area. The surface area of an aerial mycelium of the present disclosure can be characterized as the area of the aerial mycelium that occupies the plane that is substantially orthogonal to the direction of mycelial growth.

In some aspects, the surface area of an appressed mycelium of the present disclosure can be characterized as the area of the mycelium that occupies the plane substantially parallel to the direction of the mycelial growth.

In some aspects, an aerial or an appressed mycelium of the present disclosure can have a surface area that is at least about 80% of the surface area of the growth matrix, or is at least about 90% of the surface area of the growth matrix. In some further aspects, an aerial or an appressed mycelium of the present disclosure can have a surface are that is at most about 125% of the surface area of the growth matrix. In some further aspects, an aerial or an appressed mycelium of the present disclosure can have a surface area of at least about 1 square inch. In some yet further aspects, an aerial or an appressed mycelium of the present disclosure can have a surface area of at most about 2,000 square feet.

In some aspects, a mycelium of the present disclosure is characterized as a contiguous mycelium. A contiguous mycelium of the present disclosure can be obtained by removing a contiguous extra-particle mycelial growth from a growth matrix as a contiguous object.

“Contiguous” as used herein in connection with an extra-particle aerial mycelial growth or an aerial mycelium refers to an extra-particle aerial mycelial growth or an aerial mycelium having a contiguous volume, wherein the contiguous volume is at least about 15 cubic inches, has a series of linked hyphae over the contiguous volume, or both. In some embodiments, an aerial mycelium of the present disclosure can have a contiguous volume of at least about 150 cubic inches, at least about 300 cubic inches or more. In some embodiments, a contiguous aerial mycelium of the present disclosure can be obtained by removing a contiguous extra-particle aerial mycelial growth from a growth matrix as a contiguous 3-dimensional object, which may be referred to herein as a panel.

“Contiguous” as used herein in connection with an extra-particle appressed mycelial growth or an appressed mycelium refers to an extra-particle appressed mycelial growth or an appressed mycelium having anastomotic linkages, a contiguous surface area of at least about 16 cubic inches, or both. In some embodiments, the contiguous appressed mycelium can be obtained by removing a contiguous extra-particle appressed mycelial growth from a growth matrix as a contiguous sheet.

In some embodiments, a mycelium of the present disclosure is characterized as having a native density. In some embodiments, the native density is expressed as a mean native density as determined from sampling over the volume of the mycelium.

“Native density” as used herein in connection with an aerial mycelium refers to the density of an aerial mycelium having a native moisture content of at least about 80% (w/w), or at least about 90% (w/w), and at most about 100% (w/w). Typically, the native density is determined from a mycelium obtained after an incubation time period has elapsed and the resulting mycelial growth has been removed from a growth matrix, and prior to performing any optional environmental, physical or other post-processing step(s) that may compress or expand the aerial mycelium so obtained. An environmental step can be a drying step that reduces the aerial mycelial native moisture content to less than about 80% (w/w).

Thus, in some embodiments, an aerial mycelium of the present disclosure can have a mean native density of no greater than about 70 pcf. In some embodiments, an aerial mycelium of the present disclosure can have a mean native density within a range of about 0.05 pounds per square foot (pcf) to about 70 pcf. In a further embodiment, an aerial mycelium of the present disclosure can have a mean native density within a range of about 0.05 pcf to about 15 pcf. Example 23 of the present disclosure discloses a non-limiting example of an aerial mycelium having a low native density of about 0.06 pcf.

In some other embodiments, an aerial mycelium of the present disclosure can have a mean native density within a range of about 1 pcf to about 70 pcf. In some further embodiments, the aerial mycelium can have a mean native density of at least about 1 pcf, at least about 2 pcf, at least about 3 pcf, at least about 4 pcf, at least about 5 pcf, at least about 6 pcf, at least about 7 pcf, at least about 8 pcf, at least about 9 pcf or at least about 10 pcf. In yet some further embodiments, the aerial mycelium can have a mean native density of at most about 60 pcf, at most about 55 pcf, at most about 50 pcf, at most about 45 pcf, at most about 40 pcf, at most about 35 pcf, at most about 30 pcf, at most about 25 pcf, at most about 20 pcf or at most about 15 pcf. In some embodiments, an aerial mycelium of the present disclosure has a mean native density within a range of about 1 pcf to about 50 pcf, about 1 pcf to about 45 pcf, about 1 pcf to about 40 pcf, about 1 pcf to about 35 pcf, about 1 pcf to about 30 pcf, about 1 pcf to about 25 pcf, about 1 pcf to about 20 pcf, about 1 pcf to about 15 pcf, about 1 pcf to about 10 pcf, about 1 pcf to about 8 pcf, about 1 pcf to about 7 pcf, about 1 pcf to about 6 pcf, or about 1 pcf to about 5 pcf. In some further embodiments, an aerial mycelium of the present disclosure has a mean native density within a range of about 2 pcf to about 50 pcf, about 2 pcf to about 45 pcf, about 2 pcf to about 40 pcf, about 2 pcf to about 35 pcf, about 2 pcf to about 30 pcf, about 2 pcf to about 25 pcf, about 2 pcf to about 20 pcf, about 2 pcf to about 15 pcf, about 2 pcf to about 10 pcf, about 2 pcf to about 8 pcf, about 2 pcf to about 7 pcf, about 2 pcf to about 6 pcf, or about 2 pcf to about 5 pcf. In some yet further embodiments, an aerial mycelium of the present disclosure has a mean native density within a range of about 3 pcf to about 50 pcf, about 3 pcf to about 45 pcf, about 3 pcf to about 40 pcf, about 3 pcf to about 35 pcf, about 3 pcf to about 30 pcf, about 3 pcf to about 25 pcf, about 3 pcf to about 20 pcf, about 3 pcf to about 15 pcf, about 3 pcf to about 10 pcf, about 3 pcf to about 8 pcf, about 3 pcf to about 7 pcf, about 3 pcf to about 6 pcf, or about 3 pcf to about 5 pcf. In some more particular embodiments, an aerial mycelium of the present disclosure has a mean native density of about 0.05 pcf, about 1 pcf, about 2 pcf, about 3 pcf, about 4 pcf, about 5 pcf, about 6 pcf, about 7 pcf, about 8 pcf, about 9 pcf, about 10 pcf, about 11 pcf, about 12 pcf, about 13 pcf, about 14 pcf or about 15 pcf, or any range therebetween.

“Native density” as used herein in connection with an appressed mycelium refers to the density of an appressed mycelium having a native moisture content within a range of about 60% (w/w) to about 80% (w/w). Typically, the native density is determined from a mycelium obtained after an incubation time period has elapsed and the resulting mycelial growth has been removed from a growth matrix, and prior to performing any optional environmental, physical or other post-processing step(s) that may compress or expand the aerial mycelium so obtained. An environmental step can be a drying step that reduces the aerial mycelial native moisture content to less than about 60% (w/w).

In some embodiments, a mycelium of the present disclosure is characterized as having a dry density. In some embodiments, the dry density is expressed as a mean dry density as determined from sampling over the volume of the mycelium.

“Dry density” as used herein refers to the density of a mycelium having a moisture content of no greater than about 10% (w/w). Typically, the dry density of a mycelium is determined after removing mycelial growth from a growth matrix to obtain a mycelium, and subsequently drying the mycelium to a moisture content of no greater than about 10% (w/w).

Thus, in some embodiments, an aerial mycelium of the present disclosure can have a mean dry density of at most about 7 pcf, at most about 6 pcf or at most about 5 pcf. In some embodiments, an aerial mycelium of the present disclosure can have a mean dry density within a range of about 0.05 pcf to about 7 pcf, about 0.05 pcf to about 6 pcf, about 0.05 to about 5 pcf, about 0.05 to about 4 pcf, about 0.05 to about 3 pcf, about 0.1 pcf to about 7 pcf, about 0.1 to about 6 pcf, about 0.1 to about 5 pcf, about 0.1 to about 4 pcf or about 0.1 to about 3 pcf. In some further embodiments, an aerial mycelium of the present disclosure has a mean dry density within a range of about 0.1 pcf to about 2 pcf. In some more particular embodiments, an aerial mycelium of the present disclosure has a mean dry density of about 0.1 pcf, about 0.2 pcf, about 0.3 pcf, about 0.4 pcf, about 0.5 pcf, about 0.6 pcf, about 0.7 pcf, about 0.8 pcf, about 0.9 pcf, about 1.0 pcf, about 1.1 pcf, about 1.2 pcf, about 1.3 pcf, about 1.4 pcf, about 1.5 pcf, about 1.6 pcf, about 1.7 pcf, about 1.8 pcf, about 1.9 pcf or about 2 pcf, or any range therebetween.

In some aspects, an aerial mycelium of the present disclosure can be further characterized by its hyphal width. In some embodiments, an aerial mycelium of the present disclosure has a mean hyphal width of no greater than about 20 microns, or no greater than about 15 microns. In some embodiments, an aerial mycelium of the present disclosure has a mean hyphal width within a range of about 0.1 micron to about 20 microns, about 0.1 micron to about 15 microns, or about 0.2 microns to about 15 microns.

“Open volume” as used herein refers to the ratio of the volume of interstices of a mycelium to the volume of its mass, and may be referred to herein as “porosity.”

In some aspects, an aerial mycelium of the present disclosure can be characterized as having a percent porosity. In some embodiments, an aerial mycelium of the present disclosure can have a percent porosity of at least about 50% (v/v), at least about 60%, or at least about 70% (v/v). In some embodiments, an aerial mycelium of the present disclosure can have a percent porosity within a range of about 50% to about 90%, or about 60% to about 80%. In some embodiments, the aerial mycelium having said percent porosity is a dried aerial mycelium. In some further embodiments, the dried aerial mycelium has a moisture content of less than about 10% (w/w).

In some aspects, an aerial mycelium of the present disclosure can be characterized as having a median pore diameter. In some embodiments, an aerial mycelium of the present disclosure can have a median pore diameter within a range of about 10 microns to about 50 microns, about 15 microns to about 45 microns, or about 20 microns to about 35 microns.

In some embodiments, a mycelium of the present disclosure is characterized as having a Kramer shear force. “Kramer shear force” as would be readily understood by a person of ordinary skill in the art in food industry, is mechanical technique of measuring hardness and cohesiveness of food, and can be used for providing an indicator of texture (see Muscle Foods: Meat Poultry and Seafood Technology, by B.C. Breidenstein, D. M. Kinsman and A. W. Kotula; Chapter 11, Quality Characteristics; Springer Science & Business Media, Mar. 9, 2013; the entire content of which is hereby incorporated by reference in its entirety). A Kramer shear force of a material can be obtained as standard output from a Kramer shear cell test, and reported as a force-to-mass ratio, expressed in maximum kilograms of force per gram of material (kg/g). The maximum kilograms of force value can be taken from the peak of the Load-Extension curve recorded from a load cell.

Thus, in some embodiments, an aerial mycelium of the present disclosure, or an edible product containing an aerial mycelium of the present disclosure, including but not limited to an edible food product or food ingredient, can have a Kramer shear force of less than about 30 kg/g, of less than about 25 kg/g, of less than about 20 kg/g, of less than about 15 kg/g, of less than about 10 kg/g, or less than about 6 kg/g. In some further embodiments, an aerial mycelium of the present disclosure, or an edible product containing an aerial mycelium of the present disclosure, including but not limited to an edible food product or food ingredient, can have a Kramer shear force of no greater than about 5 kg/g, no greater than about 4 kg/g, no greater than about 3 kg/g or no greater than about 2 kg/g. In some further embodiments, an aerial mycelium of the present disclosure, or an edible product containing an aerial mycelium of the present disclosure, including but not limited to an edible food product or food ingredient, can have a Kramer shear force of at least about 0.1 kg/g, at least about 0.2 kg/g, at least about 0.3 kg/g, at least about 0.4 kg/g or at least about 0.5 kg/g. In yet some further embodiments, an aerial mycelium of the present disclosure, or an edible product containing an aerial mycelium of the present disclosure, including but not limited to an edible food product or food ingredient, can have a Kramer shear force of within a range of about 1 to about 15 kg/g, or within a range of about 2 to about 10 kg/g. In yet still further embodiments, an aerial mycelium of the present disclosure, or an edible product containing an aerial mycelium of the present disclosure, including but not limited to an edible food product or food ingredient, can have a Kramer shear force of about 1 kg/g, of about 2 kg/g, of about 3 kg/g, of about 4 kg/g, of about 5 kg/g, of about 6 kg/g, of about 7 kg/g, of about 8 kg/g, of about 9 kg/g, of about 10 kg/g, of about 11 kg/g, of about 12 kg/g, of about 13 kg/g, of about 14 kg/g or of about 15 kg/g, or any range therebetween.

As disclosed herein, an aerial mycelium of the present disclosure comprises a grain. As further disclosed herein, an aerial mycelium can be characterized as having a direction of growth along a first axis. Accordingly, physical properties of an aerial mycelium of the present disclosure can vary depending on how a physical (e.g., a mechanical) test or step is performed relative to the grain or to the first axis. In some non-limiting embodiments, a physical property of an aerial mycelium can be assessed in a direction parallel to the first axis, in a direction perpendicular to the first axis, or both. In other non-limiting examples, a physical property of an aerial mycelium can be assessed with the grain, against the grain, or both. Such physical properties can include Kramer shear force, ultimate tensile strength and compressive modulus, compressive stress and the like.

Thus, in some embodiments, an aerial mycelium of the present disclosure, or an edible product containing an aerial mycelium of the present disclosure, including but not limited to an edible food product or food ingredient, can have a Kramer shear force in the dimension parallel to the direction of aerial mycelial growth of no greater than about 6 kg/g, no greater than about 5 kg/g, no greater than about 4 kg/g, no greater than about 3 kg/g or no greater than about 2 kg/g. In some embodiments, an aerial mycelium of the present disclosure is characterized as having a native Kramer shear force value in the dimension parallel to the direction of aerial mycelial growth of within a range of about 1.5 kg/g to about 5.5 kg/g. In some more particular embodiments, the aerial mycelium of the present disclosure has a native Kramer shear force in the dimension parallel to the direction of aerial mycelial growth, of about 1.5 kg/g, about 1.6 kg/g, about 1.7 kg/g, about 1.8 kg/g, about 1.9 kg/g, about 2.0 kg/g, about 2.1 kg/g, about 2.2 kg/g, about 2.3 kg/g, about 2.4 kg/g, about 2.5 kg/g, about 2.6 kg/g, about 2.7 kg/g, about 2.8 kg/g, about 2.9 kg/g, about 3.0 kg/g, about 3.1 kg/g, about 3.2 kg/g, about 3.3 kg/g, about 3.4 kg/g, about 3.5 kg/g, about 3.6 kg/g, about 3.7 kg/g, about 3.8 kg/g, about 3.9 kg/g, about 4.0 kg/g, about 4.1 kg/g, about 4.2 kg/g, about 4.3 kg/g, about 4.4 kg/g, about 4.5 kg/g, about 4.6 kg/g, about 4.7 kg/g, about 4.8 kg/g, about 4.9 kg/g, about 5.0 kg/g, about 5.1 kg/g, about 5.2 kg/g, about 5.3 kg/g, about 5.4 kg/g or about 5.5 kg/g, or any range therebetween.

in some embodiments, an aerial mycelium of the present disclosure, or an edible product containing an aerial mycelium of the present disclosure, including but not limited to an edible food product or food ingredient, can have a Kramer shear force in the dimension perpendicular to the direction of aerial mycelial growth of no greater than about 9 kg/g, no greater than about 8 kg/g, no greater than about 7 kg/g, no greater than 6 kg/g, no greater than about 5 kg/g, no greater than about 4 kg/g, no greater than about 3 kg/g or no greater than about 2 kg/g. In some further embodiments, an aerial mycelium of the present disclosure can have a native Kramer shear force in the dimension perpendicular to the direction of aerial mycelial growth, of within a range of about 2.5 to about 9.0 kg/g. In some more particular embodiments, the aerial mycelium of the present disclosure has a native Kramer shear force in the dimension perpendicular to the direction of aerial mycelial growth, of about 2.5 kg/g, about 2.6 kg/g, about 2.7 kg/g, about 2.8 kg/g, about 2.9 kg/g, about 3.0 kg/g, about 3.1 kg/g, about 3.2 kg/g, about 3.3 kg/g, about 3.4 kg/g, about 3.5 kg/g, about 3.6 kg/g, about 3.7 kg/g, about 3.8 kg/g, about 3.9 kg/g, about 4.0 kg/g, about 4.1 kg/g, about 4.2 kg/g, about 4.3 kg/g, about 4.4 kg/g, about 4.5 kg/g, about 4.6 kg/g, about 4.7 kg/g, about 4.8 kg/g, about 4.9 kg/g, about 5.0 kg/g, about 5.1 kg/g, about 5.2 kg/g, about 5.3 kg/g, about 5.4 kg/g, about 5.5 kg/g, about 5.6 kg/g, about 5.7 kg/g, about 5.8 kg/g, about 5.9 kg/g, about 6.0 kg/g, about 6.1 kg/g, about 6.2 kg/g, about 6.3 kg/g, about 6.4 kg/g, about 6.5 kg/g, about, 6.6 kg/g, about 6.7 kg/g, about 6.8 kg/g, about 6.9 kg/g, about 7.0 kg/g, about 7.1 kg/g, about 7.2 kg/g, about 7.3 kg/g, about 7.4 kg/g, about 7.5 kg/g, about 7.6 kg/g, about 7.7 kg/g, about 7.8 kg/g, about 7.9 kg/g, about 8.0 kg/g, about 8.1 kg/g, about 8.2 kg/g, about 8.3 kg/g, about 8.4 kg/g, about 8.5 kg/g, about 8.6 kg/g, about 8.7 kg/g, about 8.8 kg/g, about 8.9 kg/g or about 9.0 kg/g, or any range therebetween.

In some further embodiments, an oven-dried aerial mycelium of the present disclosure can have a Kramer shear force in the dimension parallel to the direction of aerial mycelial growth, of within a range of about 50 kg/g to about 120 kg/g. In some more particular embodiments, the oven-dried aerial mycelium of the present disclosure has a Kramer shear force in the dimension parallel to the direction of aerial mycelial growth of about 50 kg/g, about 51 kg/g, about 52 kg/g, about 53 kg/g, about 54 kg/g, about 55 kg/g, about 56 kg/g, about 57 kg/g, about 58 kg/g, about 59 kg/g, about 60 kg/g, about 61 kg/g, about 62 kg/g, about 63 kg/g, about 64, kg/g, about 65 kg/g, about 66 kg/g, about 67 kg/g, about 68 kg/g, about 69 kg/g, about 70 kg/g, about 71 kg/g, about 72 kg/g, about 73 kg/g, about 74 kg/g, about 75 kg/g, about 76 kg/g, about 77 kg/g, about 78 kg/g, about 79 kg/g, about 80 kg/g, about 81 kg/g, about 82 kg/g, about 83 kg/g, about 84, kg/g, about 85 kg/g, about 86 kg/g, about 87 kg/g, about 88 kg/g, about 89 kg/g, about 90 kg/g, about 91 kg/g, about 92 kg/g, about 93 kg/g, about 94 kg/g, about 95 kg/g, about 96 kg/g, about 97 kg/g, about 98 kg/g, about 99 kg/g, about 100 kg/g, about 101 kg/g, about 102 kg/g, about 103 kg/g, about 104, kg/g, about 105 kg/g, about 106 kg/g, about 107 kg/g, about 108 kg/g, about 109 kg/g, about 110 kg/g, about 111 kg/g, about 112 kg/g, about 113 kg/g, about 114, kg/g, about 115 kg/g, about 116 kg/g, about 117 kg/g, about 118 kg/g, about 119 kg/g or about 120 kg/g, or any range therebetween.

In some embodiments, a mycelium of the present disclosure is characterized as having an ultimate tensile strength. In some embodiments, an aerial mycelium of the present disclosure has a native ultimate tensile strength of no greater than about 5 psi, no greater than about 4 psi, no greater than about 3 psi, or no greater than about 2 psi. In some embodiments, an aerial mycelium of the present disclosure has a native ultimate tensile strength of no greater than about 1.5 psi, no greater than about 1.4 psi, no greater than about 1.3 psi, no greater than about 1.2 psi or no greater than about 1.1 psi. In some embodiments, an aerial mycelium of the present disclosure has a native ultimate tensile strength of at least about 0.1 psi, at least about 0.2 psi or at least about 0.3 psi.

In some embodiments, the ultimate tensile strength of the aerial mycelia of the present disclosure can be characterized in the direction parallel to the direction of aerial mycelial growth, perpendicular to the direction of mycelial growth, or as a ratio thereof.

In some embodiments, an aerial mycelium of the present disclosure has a native ultimate tensile strength in the dimension parallel to the direction of aerial mycelial growth of no greater than about 5 psi, no greater than about 4 psi, no greater than about 3 psi, or no greater than about 2 psi. In some embodiments, an aerial mycelium of the present disclosure has a native ultimate tensile strength in the dimension parallel to the direction of aerial mycelial growth of no greater than about 1.9 psi, no greater than about 1.8 psi, no greater than about 1.7 psi, or no greater than about 1.6 psi. In some embodiments, an aerial mycelium of the present disclosure has a native ultimate tensile strength in the dimension parallel to the direction of aerial mycelial growth of at least about 0.1 psi, at least about 0.2 psi, at least about 0.3 psi, at least about 0.4 psi or at least about 0.5 psi. In some embodiments, an aerial mycelium of the present disclosure has a native ultimate tensile strength in the dimension parallel to the direction of aerial mycelial growth within a range of about 0.1 psi to about 3 psi, about 1.2 to about 2 psi, or about 0.5 psi to about 1.6 psi. In some more particular embodiments, an aerial mycelium of the present disclosure has a native ultimate tensile strength in the dimension parallel to the direction of aerial mycelial growth, of about 0.1 psi, about 0.2 psi, about 0.3 psi, about 0.4 psi, about 0.5 psi, about 0.6 psi, about 0.7 psi, about 0.8 psi, about 0.9 psi, about 1.0 psi, about 1.1 psi, about 1.2 psi, about 1.3 psi, about 1.4 psi, about 1.5 psi or about 1.6 psi, or any range therebetween.

In some embodiments, an aerial mycelium of the present disclosure has a native ultimate tensile strength in the dimension perpendicular to the direction of aerial mycelial growth of no greater than about 3 psi, no greater than about 2.5 psi, no greater than about 2 psi, no greater than about 1.5 psi, no greater than about 1 psi or no greater than about 0.5 psi. In some embodiments, an aerial mycelium of the present disclosure has a native ultimate tensile strength in the dimension perpendicular to the direction of aerial mycelial growth within a range of about 0.1 to about 2 psi, about 0.1 to about 1.5 psi, about 0.1 to about 1 psi, about 0.1 to about 0.5 psi, about 0.2 psi to about 2 psi, about 0.2 to about 1.5 psi, about 0.2 to about 1 psi, about 0.2 to about 0.5 psi, or about 0.3 psi to about 0.5 psi. In some more particular embodiments, the aerial mycelium of the present disclosure has a native ultimate tensile strength in the dimension perpendicular to the direction of aerial mycelial growth of about 0.3 psi, about 0.4 psi or about 0.5 psi, or any range therebetween.

In some embodiments, an aerial mycelium of the present disclosure has a native ultimate tensile strength in the dimension parallel to the direction of aerial mycelial growth that is at most about 5-fold greater, at most about 4-fold greater, at most about 3-fold greater, or at most about 2-fold greater than a native ultimate tensile strength in the dimension perpendicular to the direction of aerial mycelial growth. In some embodiments, an aerial mycelium of the present disclosure has a native ultimate tensile strength in the dimension parallel to the direction of aerial mycelial growth, and a native ultimate tensile strength in the dimension perpendicular to the direction of aerial mycelial growth, in a ratio of about 2:1, about 2.5:1, about 3:1, about 3.5:1 or about 4:1. In some more particular embodiments, an aerial mycelium of the present disclosure has a native ultimate tensile strength in the dimension parallel to the direction of aerial mycelial growth, and a native ultimate tensile strength in the dimension perpendicular to the direction of aerial mycelial growth, in a ratio of about 3:1.

An aerial mycelium of the present disclosure can be characterized as having an edge comprising an outer perimeter and as having a center that is interior to the edge. Thus, in some aspects, an aerial mycelium can comprise edge tissue, i.e., mycelial tissue occurring at the edge or perimeter of the aerial mycelium. In other aspects, the aerial mycelial panel can be characterized as having center tissue, i.e., tissue occurring interior to the edge of the mycelium. In a non-limiting embodiment, the center tissue comprises aerial mycelial tissue occurring interior to the edge by at least 1 inch, at least 2 inches, at least 3 inches, at least 4 inches, at least 5 or at least 6 inches from said edge. In some embodiments, an aerial mycelium of the present disclosure can be processed or “trimmed” to remove edge tissue. In a non-limiting example, an aerial mycelium (or panel) can be processed by removing up to about 1 inch, up to about 2 inches, or up to about 3 inches or more of edge tissue from the perimeter of the aerial mycelium (or panel). The amount of edge tissue to be removed can be determined based upon factors such as the volume or the physical properties of the original aerial mycelium (or panel), and/or the desired volume or physical properties of the resulting processed tissue.

In some embodiments, an aerial mycelium of the present disclosure is characterized as having a compressive modulus and a compressive stress. Aerial mycelia of the present disclosure were evaluated for compressive modulus and compressive stress using specimens obtained from edge tissue, center tissue, or both. Specimens were evaluated by compression in the direction parallel to the direction of mycelial growth, perpendicular to mycelial growth, or both.

Compressive modulus and compressive stress were determined for both edge and center tissue specimens upon compression to 10% strain in the direction parallel and perpendicular to the direction of mycelial growth. Accordingly, in some embodiments, an aerial mycelium of the present disclosure can be characterized as having a native compressive modulus at 10% strain of no greater than about 10 psi, no greater than about 5 psi, or no greater than about 4 psi. In some embodiments, an aerial mycelium of the present disclosure can be characterized as having a native compressive modulus at 10% strain of within a range of about 0.1 psi to about 5 psi, about 0.1 to about 4 psi, about 0.1 to about 3.5 psi, about 0.1 to about 3 psi, about 0.1 to about 2.5 psi, about 0.1 to about 2 psi, about 0.5 psi to about 0.7 psi, or within a range of about 0.58 psi to about 0.62 psi. In some embodiments, an aerial mycelium can be characterized as having a native compressive modulus at 10% strain of about 0.50 psi, about 0.51 psi, about 0.52 psi about 0.53 psi, about 0.54 psi, about 0.55 psi, about 0.56 psi, about 0.57 psi, about 0.58 psi, about 0.59 psi, about 0.60 psi, about 0.61 psi, about 0.62 psi, about 0.63 psi, about 0.64 psi, about 0.65 psi, about 0.66 psi, about 0.67 psi, about 0.69 psi, about 0.70 psi, about 0.8 psi, about 0.85 psi, about 0.9 psi, about 0.95 psi or about 1 psi, or any ranges therebetween. In some embodiments, an aerial mycelium can be characterized as having a mean native compressive modulus at 10% strain of no greater than about 5 psi, no greater than about 4 psi, no greater than about 3 psi or no greater than about 2 psi. In some embodiments, an aerial mycelium of the present disclosure can be characterized as having a mean native compressive modulus at 10% strain within a range of about 0.1 psi to about 1.8 psi; or of about 1 psi. In some aspects, an aerial mycelium of the present disclosure can be characterized as having a native compressive stress at 10% strain of no greater than about 1 psi. In some further embodiments, an aerial mycelium can be characterized as having a native compressive stress at 10% strain within a range of about 0.01 psi to about 0.5 psi, about 0.01 psi to about 0.4 psi, or about 0.01 psi to about 0.3 psi. In some embodiments, an aerial mycelium can be characterized as having a native compressive stress at 10% strain within a range of about 0.05 psi to about 0.15 psi, or about 0.08 psi to about 0.13 psi. In some embodiments, an aerial mycelium has a native compressive stress at 10% strain of about 0.05 psi, about 0.06 psi, about 0.07 psi, about 0.08 psi, about 0.09 psi, about 0.10 psi, about 0.11 psi, about 0.12 psi, about 0.13 psi, about 0.14 psi or about 0.15 psi, or any ranges therebetween. In some embodiments, an aerial mycelium can be characterized as having a mean native compressive stress at 10% strain of no greater than about 1 psi, no greater than about 0.5 psi, or no greater than about 0.25 psi. In some embodiments, an aerial mycelium can be characterized as having a mean native compressive stress at 10% strain within a range of about 0.01 psi to about 1 psi, about 0.01 psi to about 0.5 psi, about 0.01 psi to about 0.25, about 0.01 psi to about 0.2 psi, about 0.02 psi to about 1 psi, about 0.02 psi to about 0.5 psi, about 0.02 psi to about 0.25, or about 0.02 psi to about 0.2 psi; or of about 0.1 psi.

Compressive modulus and compressive stress were determined for both edge and center tissue specimens upon compression to 10% strain in the direction parallel to the direction of mycelial growth. Accordingly, in some embodiments, an aerial mycelium can be characterized as having a native compressive modulus at 10% strain in a direction parallel to the direction of mycelial growth of no greater than about 10 psi, no greater than about 5 psi, or no greater than about 4 psi. In some embodiments, an aerial mycelium can be characterized as having a native compressive modulus at 10% strain in a direction parallel to the direction of mycelial growth within a range of about 0.5 psi to about 5 psi, about 0.5 to about 4 psi, about 0.5 to about 3.5 psi, about 0.5 to about 3 psi, about 0.5 to about 2.5 psi, or about 0.5 to about 2 psi. In some embodiments, an aerial mycelium can be characterized as having a mean native compressive modulus at 10% strain in a direction parallel to the direction of mycelial growth of no greater than about 5 psi, no greater than about 4 psi, no greater than about 3 psi, or no greater than about 2.5 psi. In some embodiments, an aerial mycelium can be characterized as having a mean native compressive modulus at 10% strain in a direction parallel to the direction of mycelial growth within a range of about 0.1 psi to about 3 psi, about 0.2 psi to about 3 psi, about 0.3 psi to about 3 psi, about 0.4 psi to about 3 psi, about 0.5 psi to about 3 psi, about 0.5 psi to about 2.5, about 1 to about 2 psi; or of about 1.5 psi. In some further embodiments, an aerial mycelium can be characterized as having a native compressive stress at 10% strain in a direction parallel to the direction of mycelial growth of no greater than about 1 psi, no greater than about 0.5 psi, or no greater than about 0.3 psi. In some embodiments, an aerial mycelium can be characterized as having a native compressive stress at 10% strain in a direction parallel to the direction of mycelial growth within a range of about 0.01 psi to about 1 psi, about 0.01 psi to about 0.5 psi, about 0.01 psi to about 0.4 psi, or about 0.05 psi to about 0.3 psi. In some embodiments, an aerial mycelium can be characterized as having a mean native compressive stress at 10% strain in a direction parallel to the direction of mycelial growth of no greater than about 1 psi, no greater than about 0.5 psi, or no greater than about 0.25 psi. In some embodiments, an aerial mycelium can be characterized as having a mean native compressive stress at 10% strain in a direction substantially parallel to the direction of mycelial growth within a range of about 0.05 psi to about 0.25 psi, about 0.1 psi to about 0.2 psi; or of about 0.15 psi.

Compressive modulus and compressive stress were determined for both edge and center tissue specimens upon compression to 10% strain in the direction perpendicular to the direction of mycelial growth. Accordingly, in some embodiments, an aerial mycelium can be characterized as having a native compressive modulus at 10% strain in a direction perpendicular to the direction of mycelial growth of no greater than about 2 psi, no greater than about 1.5 psi, no greater than about 1 psi, or no greater than about 0.75 psi. In some embodiments, an aerial mycelium can be characterized as having a native compressive modulus at 10% strain in a direction perpendicular to the direction of mycelial growth within a range of about 0.1 psi to about 2 psi, about 0.1 psi to about 1.5 psi, about 0.1 psi to about 1 psi, or about 0.1 psi to about 0.75 psi. In some embodiments, an aerial mycelium can be characterized as having a mean native compressive modulus at 10% strain in a direction perpendicular to the direction of mycelial growth of no greater than about 1.5 psi, no greater than about 1 psi, or no greater than about 0.5 psi. In some embodiments, an aerial mycelium can be characterized as having a mean native compressive modulus at 10% strain in a direction perpendicular to the direction of mycelial growth within a range of about 0.1 psi to about 1.5 psi, about 0.1 psi to abut 1 psi, about 0.1 psi to about 0.5 psi, about 0.1 to about 0.4 psi; or of about 0.3 psi. In some further embodiments, an aerial mycelium can be characterized as having a native compressive stress at 10% strain in a direction perpendicular to the direction of mycelial growth of no greater than about 0.3 psi, no greater than about 0.2 psi, or no greater than about 0.1 psi. In some embodiments, an aerial mycelium can be characterized as having a native compressive stress at 10% strain in a direction perpendicular to the direction of mycelial growth within a range of about 0.01 to about 0.3 psi, within a range of about 0.01 to about 0.2 psi, or about 0.01 psi to about 0.1 psi. In some embodiments, an aerial mycelium can be characterized as having a mean native compressive stress at 10% strain in a direction perpendicular to the direction of mycelial growth of no greater than about 0.15 psi, or no greater than about 0.1 psi. In some embodiments, an aerial mycelium can be characterized as having a mean native compressive stress at 10% strain in a direction perpendicular to the direction of mycelial growth within a range of about 0.01 psi to about 0.15 psi, about 0.01 to about 0.1 psi; or of about 0.05 psi.

Compressive modulus and compressive stress were determined for center tissue specimens upon compression to 10% strain in the direction parallel to the direction of mycelial growth. As disclosed herein, an aerial mycelium of the present disclosure can be processed to remove edge tissue. Accordingly, in some embodiments, an aerial mycelium (or center tissue of an aerial mycelium) can be characterized as having a native compressive modulus at 10% strain in a direction parallel to the direction of mycelial growth of no greater than about 10 psi, no greater than about 9 psi, no greater than about 8 psi, no greater than about 7 psi, no greater than about 6 psi, no greater than about 5 psi, no greater than about 4 psi or no greater than about 3 psi. In some embodiments, an aerial mycelium (or center tissue of an aerial mycelium) can be characterized as having a native compressive modulus at 10% strain in a direction parallel to the direction of mycelial growth within a range of about 0.5 psi to about 10 psi, about 0.5 psi to about 7.5 psi, about 0.5 psi to about 5 psi, about 0.5 psi to about 4 psi, about 0.5 psi to about 3.5 psi, about 0.5 psi to about 3 psi, or about 1 psi to about 3 psi. In some embodiments, an aerial mycelium (or center tissue of an aerial mycelium) can be characterized as having a mean native compressive modulus at 10% strain in a direction parallel to the direction of mycelial growth of no greater than about 8 psi, no greater than about 7 psi, no greater than about 6 psi, no greater than about 5 psi, no greater than about 4 psi or no greater than about 3 psi. In some embodiments, an aerial mycelium (or center tissue of an aerial mycelium) can be characterized as having a mean native compressive modulus at 10% strain in a direction parallel to the direction of mycelial growth within a range of about 1 psi to about 5 psi, about 1 psi to about 4 psi, about 1 psi to about 3 psi; or of about 1.1 psi, about 1.2 psi, about 1.3 psi, about 1.4 psi, about 1.5 psi, about 1.6 psi, about 1.7 psi, about 1.8 psi, about 1.9 psi, about 2.0 psi, or about 2.1 psi or about 2.2 psi, or any ranges therebetween. In some further embodiments, an aerial mycelium (or center tissue of an aerial mycelium) can be characterized as having a native compressive stress at 10% strain in a direction parallel to the direction of mycelial growth of no greater than about 1 psi, no greater than about 0.9 psi, no greater than about 0.8 psi, no greater than about 0.7 psi, no greater than about 0.6 psi, no greater than about 0.5 psi, no greater than about 0.4 psi or no greater than about 0.3 psi. In some embodiments, an aerial mycelium (or center tissue of an aerial mycelium) can be characterized as having a native compressive stress at 10% strain in a direction parallel to the direction of mycelial growth within a range of about 0.05 psi to about 1 psi, about 0.05 psi to about 0.75 psi, about 0.05 to about 0.5 psi, about 0.05 psi to about 0.4 psi, about 0.05 psi to about 0.3 psi, about 0.1 psi to about 0.5 psi or about 0.1 psi to about 0.4 psi or about 0.1 psi to about 0.3 psi. In some embodiments, an aerial mycelium (or center tissue of an aerial mycelium) can be characterized as having a mean native compressive stress at 10% strain in a direction parallel to the direction of mycelial growth of no greater than about 0.8 psi, no greater than about 0.7 psi, no greater than about 0.6 psi, no greater than about 0.5 psi, no greater than about 0.4 psi or no greater than about 0.3 psi. In some embodiments, an aerial mycelium (or center tissue of an aerial mycelium) can be characterized as having a mean native compressive stress at 10% strain in a direction parallel to the direction of mycelial growth within a range of about 0.1 psi to about 0.8 psi, about 0.1 psi to about 0.7 psi, about 0.1 psi to about 0.6 psi, about 0.1 psi to about 0.5 psi, about 0.1 psi to about 0.4 psi, about 0.1 psi to about 0.3 psi, about 0.1 to about 0.25 psi, or about 0.1 psi to about 0.2 psi; or of about 0.1 psi, about 0.2 psi or about 0.3 psi, or any ranges therebetween.

Compressive modulus and compressive stress were determined for center tissue specimens upon compression to 10% strain in the direction perpendicular to the direction of mycelial growth. Accordingly, in some embodiments, an aerial mycelium (or center tissue of an aerial mycelium) can be characterized as having a native compressive modulus at 10% strain in a direction perpendicular to the direction of mycelial growth of no greater than about 2 psi, no greater than about 1.5 psi, no greater than about 1 psi, no greater than about 0.75 psi, or no greater than about 0.5 psi. In some embodiments, an aerial mycelium (or center tissue of an aerial mycelium) can be characterized as having a native compressive modulus at 10% strain in a direction perpendicular to the direction of mycelial growth within a range of about 0.1 to about 2 psi, about 0.1 to about 1.5 psi, about 0.1 to about 1 psi, about 0.1 to about 0.9 psi, about 0.1 to about 0.8 psi or about 0.1 to about 0.7 psi. In some embodiments, an aerial mycelium (or center tissue of an aerial mycelium) can be characterized as having a mean native compressive modulus at 10% strain in a direction perpendicular to the direction of mycelial growth of no greater than about 1.5 psi, no greater than about 1 psi or no greater than about 0.75 psi. In some embodiments, an aerial mycelium (or center tissue of an aerial mycelium) can be characterized as having a mean native compressive modulus at 10% strain in a direction perpendicular to the direction of mycelial growth within a range of about 0.1 psi to about 1.5 psi, about 0.1 psi to about 1 psi, about 0.1 to about 0.9 psi, about 0.1 psi to about 0.8 psi, about 0.1 psi to about 0.7 psi, or about 0.1 psi to about 0.6 psi. In some further embodiments, an aerial mycelium (or center tissue of an aerial mycelium) can be characterized as having a native compressive stress at 10% strain in a direction perpendicular to the direction of mycelial growth of no greater than about 0.5 psi, no greater than about 0.4 psi, no greater than about 0.3 psi, no greater than about 0.2 psi or no greater than about 0.1 psi. In some further embodiments, an aerial mycelium (or center tissue of an aerial mycelium) can be characterized as having a native compressive stress at 10% strain in a direction perpendicular to the direction of mycelial growth within a range of about 0.01 to about 0.5 psi, about 0.01 to about 0.4 psi, about 0.01 to about 0.3 psi, about 0.01 to about 0.2 psi, about 0.01 psi to about 0.1 psi, about 0.01 to about 0.09 psi, about 0.01 to about 0.08 psi, about 0.01 psi to about 0.07 psi, about 0.01 psi to about 0.06 psi, about 0.01 to about 0.05 psi, about 0.02 psi to about 0.1 psi, about 0.02 psi to about 0.09 psi, about 0.02 psi to about 0.08 psi, about 0.02 to about 0.07 psi, about 0.02 to about 0.06 psi, or about 0.02 to about 0.05 psi. In some embodiments, an aerial mycelium (or center tissue of an aerial mycelium) can be characterized as having a mean native compressive stress at 10% strain in a direction perpendicular to the direction of mycelial growth of no greater than about 0.3 psi, no greater than about 0.2 psi, or no greater than about 0.1 psi. In some embodiments, an aerial mycelium (or center tissue of an aerial mycelium) can be characterized as having a mean native compressive stress at 10% strain in a direction perpendicular to the direction of mycelial growth within a range of about 0.01 to about 0.3 psi, about 0.01 psi to about 0.2 psi, about 0.01 psi to about 0.1 psi, about 0.02 psi to about 0.3 psi, about 0.02 psi to about 0.2 psi, about 0.02 psi to about 0.1 psi, about 0.03 psi to about 0.3 psi, about 0.03 psi to about 0.2 psi, or about 0.03 psi to about 0.1 psi; or of about 0.02 psi, about 0.03 psi, about 0.04 psi, about 0.05 psi, about 0.06 psi or about 0.07 psi, or any ranges therebetween.

Aerial mycelia of the present disclosure can exhibit a compressive modulus upon compression in the dimension parallel to the direction of mycelial growth that exceeds the compressive modulus upon compression in the dimension perpendicular to the direction of mycelial growth. Thus, in some aspects, an aerial mycelium (or center tissue of an aerial mycelium) of the present disclosure can have a compressive modulus at 10% strain, upon compression in the dimension parallel to the direction of mycelial growth, of at least about 2-fold, at least about 3-fold, at least about 4-fold, at least about 5-fold greater than the compressive modulus at 10% strain upon compression in the dimension perpendicular to mycelial growth, or any range therebetween. In some embodiments, an aerial mycelium (or center tissue of an aerial mycelium) of the present disclosure can have a compressive modulus at 10% strain, upon compression in the dimension parallel to the direction of mycelial growth, of up to about 20-fold greater, or up to about 10-fold greater, than the compressive modulus at 10% strain, upon compression in the dimension perpendicular to mycelial growth.

Similarly, an aerial mycelium (or center tissue of an aerial mycelium) of the present disclosure can exhibit a compressive stress upon compression in the dimension parallel to the direction of mycelial growth that exceeds the compressive stress upon compression in the dimension perpendicular to the direction of mycelial growth. Thus, in some aspects, an aerial mycelium (or center tissue of an aerial mycelium) of the present disclosure can have a compressive stress at 10% strain, upon compression in the dimension parallel to the direction of mycelial growth, of at least about 2-fold, at least about 3-fold or at least about 4-fold greater than the compressive stress at 10% strain upon compression in the dimension perpendicular to mycelial growth, or any range therebetween. In some embodiments, an aerial mycelium (or center tissue of an aerial mycelium) of the present disclosure can have a compressive stress at 10% strain, upon compression in the dimension parallel to the direction of mycelial growth, of up to about 5-fold or up to about 6-fold greater than the compressive stress at 10% strain upon compression in the dimension perpendicular to mycelial growth.

Compressive stress was determined for both edge and center tissue specimens upon compression to about 65% strain, with compression in the direction perpendicular to the direction of mycelial growth. Accordingly, in some embodiments, an aerial mycelium of the present disclosure can be characterized as having a native compressive stress at 65% strain, upon compression in the direction perpendicular to the direction of mycelial growth, of no greater than about 10 psi, no greater than about 5 psi, no greater than about 1 psi, or no greater than about 0.5 psi. In some embodiments, an aerial mycelium of the present disclosure can be characterized as having a native compressive stress at 65% strain, upon compression in the direction perpendicular to the direction of mycelial growth within a range of about 0.01 psi to about 1 psi, about 0.01 psi to about 0.5 psi, about 0.02 psi to about 1 psi, about 0.02 psi to about 0.5 psi, about 0.03 psi to about 1 psi, about 0.03 psi to about 0.5 psi, or about 0.03 psi to about 0.4 psi. In some embodiments, an aerial mycelium of the present disclosure can be characterized as having a mean native compressive stress at 65% strain, upon compression in the direction perpendicular to the direction of mycelial growth, of no greater than about 10 psi, no greater than about 5 psi, no greater than about 1 psi, or no greater than about 0.5 psi. In some embodiments, an aerial mycelium of the present disclosure can be characterized as having a mean native compressive stress at 65% strain, upon compression in the direction perpendicular to the direction of mycelial growth, within a range of about 0.01 psi to about 1 psi, about 0.01 psi to about 0.5 psi, about 0.02 psi to about 1 psi, about 0.02 psi to about 0.5 psi, about 0.03 psi to about 1 psi, about 0.03 psi to about 0.5 psi, about 0.04 psi to about 1 psi, or about 0.04 to about 0.5 psi.

Compressive stress was determined for center tissue specimens upon compression to about 65% strain, with compression in the direction perpendicular to the direction of mycelial growth. Accordingly, in some embodiments, an aerial mycelium (or center tissue of an aerial mycelium) of the present disclosure can be characterized as having a native compressive stress at 65% strain, upon compression in the direction perpendicular to the direction of mycelial growth, of no greater than about 10 psi, no greater than about 5 psi, no greater than about 1 psi, no greater than about 0.5 psi, or no greater than about 0.25 psi. In some embodiments, an aerial mycelium (or center tissue of an aerial mycelium) of the present disclosure can be characterized as having a native compressive stress at 65% strain, upon compression in the direction perpendicular to the direction of mycelial growth, within a range of about 0.01 psi to about 1 psi, about 0.01 psi to about 0.5 psi, about 0.01 psi to about 0.25 psi, about 0.02 psi to about 1 psi, about 0.02 psi to about 0.5 psi, about 0.02 psi to about 0.25 psi, about 0.03 psi to about 1 psi, about 0.03 psi to about 0.5 psi, about 0.03 psi to about 0.25 psi, about 0.04 psi to about 1 psi, about 0.04 to about 0.5 psi, or about 0.04 to about 0.25 psi. In some embodiments, an aerial mycelium (or center tissue of an aerial mycelium) of the present disclosure can be characterized as having a mean native compressive stress at 65% strain, upon compression in the direction perpendicular to the direction of mycelial growth, of no greater than about 10 psi, no greater than about 5 psi, no greater than about 1 psi, no greater than about 0.5 psi, or no greater than about 0.25 psi. In some embodiments, an aerial mycelium (or center tissue of an aerial mycelium) of the present disclosure can be characterized as having a mean native compressive stress at 65% strain, upon compression in the direction perpendicular to the direction of mycelial growth, within a range of about 0.01 psi to about 1 psi, about 0.01 psi to about 0.5 psi, about 0.01 psi to about 0.25 psi, about 0.02 psi to about 1 psi, about 0.02 psi to about 0.5 psi, about 0.02 psi to about 0.25 psi, about 0.02 psi to about 0.2 psi, about 0.03 psi to about 1 psi, about 0.03 psi to about 0.5 psi, about 0.03 psi to about 0.25 psi, about 0.03 psi to about 0.2 psi, about 0.04 psi to about 1 psi, about 0.04 to about 0.5 psi, about 0.04 to about 0.25 psi, about 0.04 to about 0.2 psi, about 0.05 psi to about 1 psi, about 0.05 to about 0.5 psi, about 0.05 to about 0.25 psi or about 0.05 psi to about 0.2 psi, or about 0.05 psi to about 0.15 psi.

In some aspects, the present disclosure provides for an edible mycelium-based food product or an edible mycelium-based food ingredient.

“Edible” as used herein refers to being generally regarded as safe to be eaten by humans, especially after cooking; being generally considered palatable by humans; and/or being capable of being substantially masticated by humans.

“Mycelium-based” as used herein refers to a composition substantially comprising mycelium.

An edible mycelium-based food product or food ingredient can be distinguished from a mycelium-based medicine or from a mycelium-based nutritional supplement upon consideration of factors such as the method, form and/or quantity for ingestion.

In some embodiments, an edible mycelium-based product or ingredient of the present disclosure can exclude a mycelium-based medicine. In some other embodiments, an edible mycelium-based product or ingredient of the present disclosure can exclude a mycelium-based nutritional supplement.

In some aspects, the present disclosure provides for an aerial mycelium characterized by its native nutritional content. As used herein, “native nutritional content” refers to the nutritional content of an aerial mycelium obtained after an incubation time period has elapsed and the resulting mycelial growth has been removed from a growth matrix, and prior to performing any optional environmental, physical or other post-processing step(s) that may substantially alter the nutritional content of the aerial mycelium so obtained. Non-limiting examples of native nutritional content include native protein content, native fat content, native carbohydrate content, native dietary fiber content, native vitamin content, native mineral content, and so on. Typically, the nutritional content is reported based on the dry weight of the mycelium (see Example 34).

Thus, in some aspects, an aerial mycelium of the present disclosure is characterized as having a native protein content. In some embodiments, an aerial mycelium of the present disclosure is characterized as having a native protein content of at least about 20% (w/w), or at least about 25% (w/w), on a dry weight basis. In some further embodiments, an aerial mycelium of the present disclosure is characterized as having a native protein content of at most about 50% (w/w), or at most about 45% (w/w), on a dry weight basis. In some embodiments, an aerial mycelium of the present disclosure is characterized as having a native protein content within a range of about 20% to about 50% (w/w), about 21% to about 49% (w/w), about 22% to about 48% (w/w), about 23% to about 47%, about 24% to about 46% (w/w), about 25% to about 45% (w/w), about 26% to about 44% (w/w), about 27% to about 43% (w/w) or about 28% to about 42% (w/w), on a dry weight basis. In some more particular embodiments, an aerial mycelium of the present disclosure is characterized as having a native protein content of about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w) or about 50% (w/w), on a dry weight basis.

In some aspects, an aerial mycelium of the present disclosure is characterized as having a native fat content. As used herein, native fat content refers to native triglyceride content, and can be determined according to methods known to persons of ordinary skill in the art. In a non-limiting example, the fat content is determined according to Example 34C. In some embodiments, an aerial mycelium of the present disclosure is characterized as having a native fat content of at most about 7% (w/w), or at most about 6% (w/w), on a dry weight basis. In some further embodiments, an aerial mycelium of the present disclosure is characterized as having a native fat content of at least about 1% (w/w), at least about 1.5% (w/w), at least about 2% (w/w), at least about 2.5% w/w) or at least about 3% (w/w), on a dry weight basis. In yet some further embodiments, an aerial mycelium of the present disclosure is characterized as having a native fat content within a range of about 1% (w/w) to about 7% (w/w), or about 1.5% to about 6.5% (w/w), on a dry weight basis. In some more particular embodiments, an aerial mycelium of the present disclosure is characterized as having a native fat content of about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5.0% (w/w), about 5.1% (w/w), about 5.2% (w/w), about 5.3% (w/w), about 5.4% (w/w), about 5.5% (w/w), about 5.6% (w/w), about 5.7% (w/w), about 5.8% (w/w), about 5.9% (w/w), about 6.0% (w/w), about 6.1% (w/w), about 6.2% (w/w), about 6.3% (w/w), about 6.4% (w/w), about 6.5% (w/w), about 6.6% (w/w), about 6.7% (w/w), about 6.8% (w/w), about 6.9% (w/w) or about 7.0% (w/w), on a dry weight basis.

In some aspects, an aerial mycelium of the present disclosure is characterized as having a native carbohydrate content. In some embodiments, an aerial mycelium of the present disclosure is characterized as having a native carbohydrate content of at least about 30% (w/w), or at least about 35% (w/w), on a dry weight basis. In some further embodiments, an aerial mycelium of the present disclosure is characterized as having a native carbohydrate content of at most about 60% (w/w), or at most about 55% (w/w), on a dry weight basis. In some embodiments, an aerial mycelium of the present disclosure is characterized as having a native carbohydrate content within a range of about 30% (w/w) to about 60% (w/w), about 35% (w/w) to about 55% (w/w), about 40% (w/w) to about 55% (w/w), about 40% (w/w) to about 50% (w/w), or about 45% (w/w) to about 55% (w/w), on a dry weight basis. In some more particular embodiments, an aerial mycelium of the present disclosure is characterized as having a native carbohydrate content of about 30% (w/w), about 31% (w/w), about 32% (w/w), about 33% (w/w), about 34% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w) or about 60% (w/w), on a dry weight basis.

In some aspects, an aerial mycelium of the present disclosure is characterized as having a native inorganic content. As used herein, native inorganic content is reported based on ash content, which can be determined according to methods known to persons of ordinary skill in the art. In a non-limiting example, the ash content is determined according to Example 34F. In some embodiments, an aerial mycelium of the present disclosure is characterized as having a native inorganic content of at least about 5% (w/w), at least about 6% (w/w), at least about 7% (w/w), at least about 8% (w/w) or at least about 9% (w/w), or at least about 10% (w/w), on a dry weight basis. In some further embodiments, an aerial mycelium of the present disclosure is characterized as having a native inorganic content of at most about 20% (w/w), on a dry weight basis. In some embodiments, an aerial mycelium of the present disclosure is characterized as having a native inorganic content within a range of about 5% (w/w) to about 20% (w/w), about 6% (w/w) to about 20% (w/w), about 7% (w/w) to about 200 (w/w), about 8% (w/w) to about 20% (w/w), about 9% (w/w) to about 20% (w/w), about 10% (w/w) to about 20% (w/w), or about 9% (w/w) to about 18% (w/w), on a dry weight basis. In some more particular embodiments, an aerial mycelium of the present disclosure is characterized as having a native inorganic content of about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w) or about 10% (w/w), on a dry weight basis.

In some aspects, an aerial mycelium of the present disclosure is characterized as having a native dietary fiber content. In some embodiments, an aerial mycelium of the present disclosure is characterized as having a native dietary fiber content of at least about 15% (w/w), on a dry weight basis. In some further embodiments, an aerial mycelium of the present disclosure is characterized as having a native dietary fiber content of at most about 35% (w/w), on a dry weight basis. In some embodiments, an aerial mycelium of the present disclosure is characterized as having a native dietary fiber content within a range of about 15% (w/w) to about 35% (w/w), on a dry weight basis. In some more particular embodiments, an aerial mycelium of the present disclosure is characterized as having a native dietary fiber content of about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 310% (w/w), about 32% (w/w), about 33% (w/w), about 34% (w/w) or about 35% (w/w), on a dry weight basis.

In some aspects, the present disclosure provides for an aerial mycelium having a native potassium content of at least about 4000 milligrams of potassium per 100 grams of dry aerial mycelium. In some embodiments, an aerial of the present disclosure has a native potassium content within a range of about 4000 mg potassium per 100 g dry aerial mycelium to about 7000 mg potassium per 100 g dry aerial mycelium. In some further embodiments, an aerial of the present disclosure has a native potassium content within a range of about 4500 mg potassium per 100 g dry aerial mycelium to about 6500 mg potassium per 100 g dry aerial mycelium.

In some embodiments, there is provided a batch of aerial mycelia.

“Batch” as used herein refers to a quantity of goods produced at one time, wherein the quantity is at least two (2). In some embodiments, the quantity is at most about 10,000, at most about 5,000, at most about 1000, at most about 500, at most about 100, at most about 50, at most about two dozen or at most about a dozen. A batch of edible aerial mycelia of the present disclosure can be produced in a growth chamber or other system configured for growing edible aerial mycelia, or another controlled growth environment. In some embodiments, a batch of edible aerial mycelia of the present disclosure is produced under a predetermined set of growth conditions.

Thus, in some embodiments, there is provided a batch of aerial mycelia (or aerial mycelial panels). In some embodiments, greater than 50% of the aerial mycelia (or panels) in said batch conform to having one or more properties. Non-limiting examples of said properties include a native density, a native moisture content, a native thickness, a native volume, absence of a fruiting body, a native compressive modulus, a native compressive stress, a native ultimate tensile strength and/or a native Kramer shear force, wherein each said property can have a preestablished value or range of values. In some further embodiments, greater than 50% of the aerial mycelia (or panels) in the batch conform to at least two, at least three, at least four, at least five, at least six or more of said properties. In some embodiments, at least about 75% or more of the aerial mycelia (or panels) in a batch confirm to having at least one, two, three, four, five, six or more of said properties. In some embodiments, an aerial mycelium of a batch of aerial mycelia (or panels) can have one or more of said properties that are predetermined, e.g., by establishing a set of growth conditions and target values or ranges of values prior to making the aerial mycelium or batch of aerial mycelia.

As disclosed herein, a growth matrix of the present disclosure comprises a substrate to support mycelial growth. A variety of substrates are suitable to support the growth of an edible aerial mycelium or an edible appressed mycelium of the present disclosure. Suitable substrates are disclosed, for example, in US20200239830A1, the entire contents of which are hereby incorporated by reference in their entirety. In some embodiments, the substrate is a natural substrate. Non-limiting examples of a natural substrate include a lignocellulosic substrate, a cellulosic substrate or a lignin-free substrate. A natural substrate can be an agricultural waste product or one that is purposefully harvested for the intended purpose of food production, including mycelial-based food production. Further non-limiting examples of substrate(s) suitable for supporting the growth of edible mycelia of the present disclosure include soy-based materials, oak-based materials, maple-based materials, corn-based materials, seed-based materials and the like, or combinations thereof. The materials can have a variety of particle sizes, as disclosed in US20200239830A1, and occur in a variety of forms, including shavings, pellets, chips, flakes or flour, or can be in monolithic form. Non-limiting examples of suitable substrates for the production of edible mycelia of the present disclosure include corn stover, maple flour, maple flake, maple chips, soy flour, chick pea flour, millet seed flour, oak pellets, soybean hull pellets and combinations thereof. Additional useful substrates for the growth of edible mycelia are disclosed herein. Any suitable substrate can be used alone, or optionally combined with a further source of nutrition (e.g., a nutritional supplement), as media to support mycelial growth. The growth media can be hydrated to a final moisture content of greater than or equal to 50% (w/w), which can occur prior to inoculation with a fungal inoculum. In a non-limiting example, the substrate or growth media can be hydrated to a final moisture content within a range of about 50% (w/w) to about 75% (w/w), or within a range of about 60% (w/w) to about 70% (w/w).

In some aspects, the present disclosure provides for methods of processing a mycelium of the present disclosure. These post-processing methods, as described herein, can be used to modify a mycelium, including an aerial mycelium, to provide an edible food ingredient scaffold or food product, such as a panel, slab or strips, of mycelium-based bacon. This post-processing can include steps such as cutting, slicing, pressing and/or perforating. The post-processing can include amending the mycelium through boiling, brining, drying, fatting and/or the incorporation of additives. The post-processing of the mycelium provides a mycelium-based product that more closely resembles animal tissue. Any number of steps or combinations of steps can be performed in any variety of sequences to achieve the desired result. Methods of processing mycelial tissue are disclosed in US2020/0024557A1, the entire contents of which are hereby incorporated by reference in their entirety.

As disclosed herein, an aerial mycelium of the present disclosure can be obtained as a contiguous 3-dimensional object, such as a panel. Thus, an aerial mycelium or a panel or slab thereof can be further characterized by its volume. In some embodiments, the volume of an aerial mycelium (or panel) can be characterized by its thickness, such as its native thickness. In some further embodiments, the aerial mycelial volume can be characterized by its surface area. As such, the surface area of an aerial mycelial (or panel) can be further characterized as having a length and a width.

In some aspects, an aerial mycelium of the present disclosure can be compressed to form a higher density material. The mycelium can be compressed in any direction, such as with the grain or against the grain.

In some aspects, an aerial mycelium can be compressed in a direction substantially non-parallel with respect to the aerial mycelial growth axis (first axis) to form a compressed mycelium.

A compressed mycelium can have a fractional anisotropy that is substantially the same as that of the original aerial mycelium prior to the compression, or can have a higher percentage of fractional anisotropy as compared to the original aerial mycelium prior to the compression. In some embodiments, a compressed mycelium can have a fractional anisotropy of at least about 10%, or at least about 15%. In some embodiments, a compressed mycelium can have a fractional anisotropy that is substantially greater than that of the original aerial mycelium prior to the compression. Conversely, an aerial mycelium of the present disclosure can have a fractional anisotropy that is substantially less than that of the compressed mycelium.

The compressing can be completed on an aerial mycelium, for example, on a panel or section (as described further below), to form a compressed panel or section, respectively. The compressing can be completed with the compression force applied in a compressing direction which is substantially non-parallel with respect to the first axis. In some embodiments, the panel or section is compressed in a compressing direction relative to the first axis which is within a range of greater than 45 degrees and less than 135 degrees, for example, greater than about 70 degrees and less than about 110 degrees, or greater than about 80 degrees and less than about 100 degrees, with respect to the first axis. In some embodiments, the compressing direction is substantially orthogonal to the first axis.

In some aspects, compressing comprises applying force to a panel, section or strip. The force can be applied via physical impact, via a static or dynamic load. In some embodiments, mechanical force, including pneumatic or hydraulic force, can be applied, for example, via a mechanical press, such as a hydraulic press or pneumatic press. The compressing can reduce the volume and increase the density of the panel, section or strip.

In some embodiments, compressing comprises constraining a panel, section or strip during said compression. In some embodiments, constraining comprises constraining a first dimension of a panel (or a section or strip) that is substantially perpendicular to the grain (or first axis), and further constraining a second dimension that is both substantially parallel to the grain (or first axis) and substantially perpendicular to the compressing direction; consequently, a native panel thickness can be retained. In a non-limiting example, an aerial mycelium is constrained such that it's native thickness and its width are constrained during compression, such that its length is reduced via the compression. In some embodiments, an aerial mycelium can be compressed to within a range of about 15 to about 75° % of its original length or width. In some further embodiments, the aerial mycelium can be compressed to within a range of about 30% to about 40% of its original length or width.

In some aspects, compressing an aerial mycelium comprises applying a force to an aerial mycelium (e.g., a panel, a section or a strip) that is less than the force required to shear the aerial mycelium (e.g., the panel, section or strip).

In some embodiments, compressing an aerial mycelial panel, at least one section or at least one strip, can provide a compressed panel, section or strip, respectively, having a compressive stress at 65% strain of less than about 10 psi, less than about 1 psi or less than about 0.5 psi. Thus, in some embodiments, the present disclosure provides for a compressed panel, at least one compressed section or at least one compressed strip characterized as having a compressive stress at 65% strain of less than about 10 psi. In some embodiments, a compressed panel, an at least one compressed section or an at least one compressed strip can be characterized as having a compressive stress at 65% strain of less than about 1 psi. In some embodiments, a compressed panel, an at least one compressed section or an at least one compressed strip can be characterized as having a compressive stress at 65% strain of at most about 0.5 psi.

An aerial mycelium or a compressed mycelium of the present disclosure can be further processed by forming one or more sections and/or one or more strips. To form one or more sections or strips, the mycelium or compressed mycelium can be cut in any direction, such as with the grain or against the grain. In a non-limiting example, an aerial mycelium can be cut against the grain to provide a thinner panel (e.g., an aerial mycelium having a mean native thickness of about 80 mm can be cut against the grain to provide two panels, each having a mean thickness of about 40 mm).

As it is an object of the present disclosure to provide a food product or ingredient having the look and mouth-feel of a whole cut of meat (e.g., a whole muscle meat alternative), it can be important to retain the mycelial grain, in whole or at least in part Thus, in some aspects, a post-processing method can exclude cutting, shearing, grinding and/or “mincing” a mycelium, or more particularly, can exclude cutting, shearing, grinding and/or “mincing” a mycelium against the grain. In some embodiments, a post-processing method can exclude an extrusion step. Thus, in some embodiments, a food product or ingredient of the present disclosure can exclude an extruded, ground and/or minced mycelium-based product. In some embodiments, a post-processing method of the present disclosure can comprise cutting an aerial mycelium with the grain.

In some aspects, an aerial mycelium, or a compressed mycelium, of the present disclosure can be sectioned by cutting a panel of aerial mycelium, or a compressed mycelium (e.g. compressed panel), to form one or more sections, or one or more compressed sections, respectively. In some aspects, an aerial mycelium or a compressed mycelium is cut in a cutting direction substantially parallel with respect to the first axis. In some embodiments, the aerial mycelium or a compressed mycelium (e.g panel) is cut in a cutting direction within a range of plus or minus 45 degrees with respect to the first axis, for example, within a range of plus or minus about 30 degrees with respect to the first axis, or within a range of plus or minus about 15 degrees with respect to the first axis, or within a range of plus or minus about 10 degrees, 5 degrees, 3 degrees, or 1 degree with respect to the first axis, or any range therebetween.

An aerial or compressed mycelium, or a section thereof, can be further processed into strips. In some aspects, an aerial mycelium (e.g, panel) or compressed mycelium (e g. compressed panel), or section thereof is cut in a cutting direction substantially parallel with respect to the first axis to provide at least one strip or at least one compressed strip. In some embodiments, an aerial or compressed mycelium or section thereof is cut in a cutting direction within a range of plus or minus 45 degrees with respect to the first axis, for example, within a range of plus or minus about 30 degrees with respect to the first axis, or within a range of plus or minus about 15 degrees with respect to the first axis, or within a range of plus or minus about 10 degrees, 5 degrees, 3 degrees, or 1 degree with respect to the first axis, or any range therebetween, to provide at least one strip or at least one compressed strip.

Cutting can be achieved by a variety of means, including but not limited to cutting with a knife, a meat or deli slicer, a bacon slicer, an ultrasonic cutter, a water jet cutter, a bandsaw and the like.

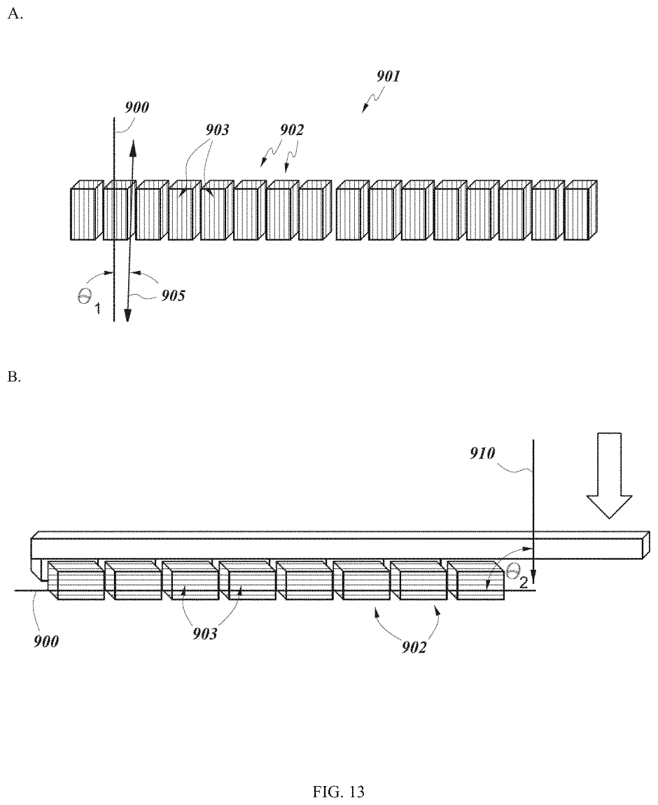

FIGS. 13A and 13B illustrate examples of the aforementioned cutting and compressing steps, and relative angular orientations, for an aerial mycelium 901. The aerial mycelium 901 is characterized as having a direction of mycelial growth along an axis 900, as shown by grains 903. For example, FIG. 13A illustrates an aerial mycelium 901 which has been sectioned by cutting the aerial mycelium 901, to form one or more sections 902. The sections 902 were formed by cutting the aerial mycelium or a compressed mycelium in a cutting direction 905 at an angle θ1 which is substantially parallel with respect to the axis 900. The cutting step shown in FIG. 13A can be implemented before or after a compression step. For example, the cutting step can be implemented on a compressed or uncompressed mycelium, e.g., a compressed or uncompressed panel, respectively, to form sections 902.

FIG. 13B illustrates compressing the sections 902 in a compressing direction 910 at an angle θ2 which is substantially non-parallel with respect to the axis 900. The compressing step shown in FIG. 13B can be implemented before or after a cutting step. For example, the compression step can be implemented to compress sections 902, as shown, or can be performed on the aerial mycelium 901 prior to forming sections 902. Multiple compression and cutting steps can be performed in a sequence, for example, the aerial mycelium can be cut to form a section, and the section can be cut to form strips, and so forth, with one or more compression steps implemented before or after the cutting steps within this sequence.

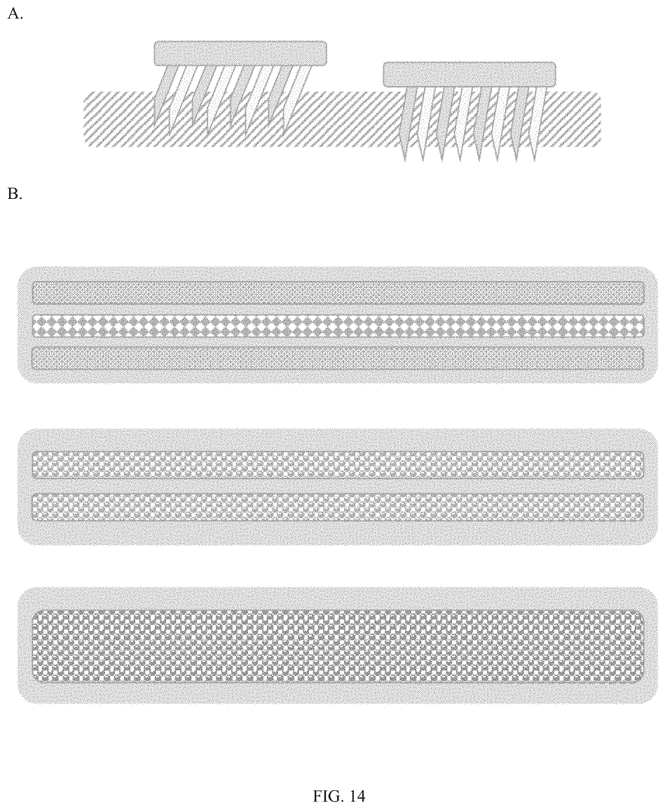

In some aspects, the present disclosure provides for perforating a mycelium, including an aerial mycelium, such as a panel, a section or a strip, or a compressed panel, section or strip. In some aspects, a perforating step is to disrupt mycelial tissue network, modify texture, form a mycelium that more closely mimics animal tissue in appearance and/or mouth-feel, and/or cooks at different rates. In some embodiments, perforating can include needling. Thus, one or more needles or the like can be inserted to penetrate the outer surface of a mycelium (e g., a panel, section, strip or a compressed panel, section or strip) (see, e.g., the left side of FIG. 14A), and/or can be inserted through the entire tissue (see, e.g., the right side of FIG. 14A). Perforating can be varied in density, intensity and shape (see, e.g., FIG. 14B), and/or by using needles of various gauges and shapes (e.g., straight or barbed) across the matrix to disrupt tissue network, and create sections that cook at different rates than others, modifying finished texture.

In some aspects, the present disclosure provides for post-processing steps via one or more amending steps to amend a mycelium, such as boiling, brining, drying and/or fatting. Chemical and/or enzymatic methods can be used to amend the mycelial tissue, for example, as described in US2020/0024557A1.

Accordingly, a mycelium (e.g, an aerial mycelium) of the present disclosure (or any section or strip obtained therefrom, or any compressed and/or perforated panel, section or strip) can be boiled. In some aspects, the boiling is to reduce moisture, modify or denature proteins, disinfect, reduce or remove native compounds and/or maloders, and/or reduce bitterness. In a non-limiting example, a mycelium (or any section or strip obtained therefrom, or any compressed and/or perforated panel, section or strip) can be boiled to remove volatile compounds, anti-nutrients or both. In some embodiments, a volatile compound can include a polyphenolic compound. In some embodiments, an anti-nutrient can include a lysin, a lectin, or both. In some embodiments, a boiling step comprises boiling an aerial mycelium of the present disclosure (or any section or strip obtained therefrom, or any compressed and/or perforated panel, section or strip) in an aqueous solution. In some embodiments, the aqueous solution comprises one or more additives. In some more particular embodiments, the aqueous solution contains salt. In some embodiments, the aqueous solution can have a salt concentration of at most about 26% (w/w) (i.e., a saturated saline solution). In some embodiments, the salt concentration is within a range of about 0.1% (w/w) to about 26% (w/w), about 0.1% to about 15% (w/w), about 0.5% to about 10% (w/w), about 0.5% to about 5% (w/w) or about 1% to about 3%. In some embodiments, the salt is sodium chloride. Other additives can include but are not limited to flavorants and/or colorants. The time and/or temperature of the boiling and the concentration of the salt and any additives can be adjusted by the skilled person to achieve the desired salt content, additive content, moisture content, protein denaturization, sterility, native compound and/or malodors content or the like in the resulting boiled composition or final product.

In some aspects, a mycelium (e.g., an aerial mycelium or panel) of the present disclosure (or any section or strip obtained therefrom, or any compressed and/or perforated panel, section or strip, or any boiled panel, section or strip) can be brined, for example, to impart flavor and/or color. In some embodiments, a brining step can include contacting an aerial mycelium (e.g., panel, or any section or strip obtained therefrom, or any compressed and/or perforated panel, section or strip, or any boiled panel, section or strip) with a brine fluid. In some embodiments, a brine fluid can be an aqueous solution containing salt. In some embodiments, the aqueous salt solution can have a salt concentration of at most about 26% (w/w) (i.e., a saturated saline solution). In some embodiments, the salt concentration is within a range of about 0.1% (w/w) to about 26% (w/w), about 0.1% to about 15% (w/w), about 0.5% to about 10% (w/w), about 0.5% to about 5% (w/w) or about 1% to about 3%. In some embodiments, the salt is sodium chloride. In some embodiments, the brine fluid comprises one or more additives. In some embodiments, the one or more additives includes flavorants and/or colorants. A brining step can comprise soaking, marinating or simmering a mycelium (e.g., an aerial mycelium or panel) of the present disclosure (or any section or strip obtained therefrom, or any compressed and/or perforated panel, section or strip, or any boiled panel, section or strip) in the brine fluid, or can comprise injecting or topically applying the brine fluid. The time and/or temperature of the brining and the concentration of the salt and any further additives can be adjusted by the skilled person to achieve the desired salt and additive content in the resulting brined composition or final product.

In some aspects, a mycelium (e.g., an aerial mycelium) of the present disclosure (or any section or strip obtained therefrom, or any compressed and/or perforated panel, section or strip, or any boiled panel, section or strip, or any brined panel, section or strip) can be dried. In some embodiments, a drying step can include heating a mycelium (e.g., an aerial mycelium) of the present disclosure (or any section or strip obtained therefrom, or any compressed and/or perforated panel, section or strip, or any boiled panel, section or strip, or any brined panel, section or strip. The drying or more particularly, the heating, can be performed by any variety of means, including a conventional oven, a convection oven, a microwave, a dehydrator or a freeze dryer or the like. The drying time and means can be adjusted by the skilled person to achieve the desired moisture content of the resulting dried composition or final product.

In some aspects, a mycelium (e.g., an aerial mycelium) of the present disclosure, or any section or strip obtained therefrom, or any compressed and/or perforated panel, section or strip, or any boiled panel, section or strip, or any brined panel, section or strip, each of which is optionally dried, can be fatted. In some embodiments, a fatting step can include contacting a mycelium (e.g., an aerial mycelium) of the present disclosure, or any section or strip obtained therefrom, or any compressed and/or perforated panel, section or strip, or any boiled panel, section or strip, or any brined panel, section or strip, each of which is optionally dried, with a fat. Non-limiting embodiments of fatting include marinating, confitting, injecting or topically applying the fat. Non-limiting examples of a fat are disclosed herein. In some embodiments, the fat further comprises an additive, including but not limited to a colorant, flavorant, or both. After adding the fat, the fatted mycelial tissue can be cooled to set the fat. The cooling step can include refrigeration of the fatted tissue.

Any number of combinations of processing steps can be implemented, such as cutting, compressing, boiling, brining, and/or fatting, and so on, to provide a cut, compressed, boiled, brined and/or fatted mycelium. In a non-limiting example, a strip of aerial mycelium, having been processed via brining and fatting, can be referred to herein as a brined, fatted strip. In another non-limiting example, a strip of aerial mycelium, having been processed via compressing (prior to or after a cutting step), brining and fatting, can be referred to herein as a compressed, brined, fatted strip.

In some aspects, the present disclosure provides for the incorporation of one or more additives into the mycelial tissue or onto the surface of the mycelial tissue. The additive can be incorporated during or after the growth of the mycelium, and before, during or after any one or more post-processing steps. Additives suitable for the incorporation into a mycelium of the present disclosure and methods of incorporating the same are disclosed in US2020/0024557A1. Additional useful additives for incorporation into edible mycelia of the present disclosure, and methods of incorporation thereof, are disclosed herein.

In some aspects, an additive can be a fat, a protein, a peptide, an amino acid, a flavorant, an aromatic agent, a mineral, a vitamin, a micronutrient, a colorant or a preservative; or a combination thereof. An additive can be a naturally occurring additive or an artificial additive, or a combination thereof.

Non-limiting examples of a fat include almond oil, animal fat, avocado oil, butter, canola oil (rapeseed oil), coconut oil, corn oil, grapeseed oil, hempseed oil, lard, mustard oil, olive oil, palm oil, peanut oil, rice bran oil, safflower oil, soybean oil, sunflower seed oil, vegetable oil, or vegetable shortening; or a combination thereof. In some embodiments, the fat is a plant-based oil or fat. In some embodiments, the plant-based oil is coconut oil or avocado oil. In some embodiments, the oil is a refined oil. In some embodiments, the fat is animal fat. In some embodiments, the animal fat is pork fat, chicken fat or duck fat.

Non-limiting examples of a flavorant include a smoke flavorant, umami, maple, a salt, a sweetener, a spice, or a meat flavor (e.g., pork flavor); or a combination thereof.

Non-limiting examples of a smoke flavorant include applewood flavor, hickory flavor, liquid smoke; or a combination thereof.

Non-limiting examples of umami include a glutamate, such as sodium glutamate.

Non-limiting examples of a salt include sodium chloride, table salt, flaked salt, sea salt, rock salt, kosher salt or Himalayan salt; or a combination thereof.

Non-limiting examples of a sweetener include sugar, cane sugar, brown sugar, honey, molasses, juice, nectar, or syrup; or a combination thereof.

Non-liming examples of a colorant include beet extract, beet juice, or paprika; or a combination thereof.

Non-limiting examples of a spice include paprika, pepper, mustard, garlic, chili, jalapeno, and the like; or a combination thereof.

“Aromatic agent” as used herein refers to a substance having a distinctive fragrance. Non-liming examples of an aromatic agent include allicin.

Non-limiting examples of a mineral include iron, magnesium, manganese, selenium, zinc, calcium, sodium, potassium, molybdenum, iodine or phosphorus; or a combination thereof.

Non-limiting examples of a vitamin include ascorbic acid (vitamin C), biotin, a retinoid, a carotene, vitamin A, thiamine (vitamin B1), riboflavin (vitamin B2), niacin (vitamin B3), pantothenic acid (vitamin B5), pyridoxine (vitamin B6), folate, folic acid (vitamin B9), cobalamine (vitamin B12), choline, calciferol (vitamin D), alpha-tocopherol (vitamin E) or phylloquinone (menadione, vitamin K); or a combination thereof.

Non-limiting examples of a protein include a plant-derived protein, a heme protein; or a combination thereof.

Non-limiting examples of an amino acid include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine; or a combination thereof.

One or more additives can be incorporated into a mycelium of the present disclosure at virtually any step(s) during or between the mycelium growth or post-processing steps described herein.

In some embodiments, one or more additives can be included in (e.g., admixed with) a growth matrix, growth media, growth media substrate, and/or in a further source of nutrition (e.g., a nutritional supplement) in the growth media.

As disclosed in US2020/0024557A1, an additive can be deposited on the growth media during the growth process, either through liquid or solid deposition, or though natural cellular uptake (bioadsorbtion), e.g., increasing mineral content in the growth media, to increase final content in the panel of tissue. Furthermore, during growth, desired nutrients, flavors, or other additives can be aerosolized into the growth chamber, condense on the propagating tissue, and be incorporated into the matrix.

As disclosed herein, an aerial mycelium of the present disclosure can be obtained by depositing aqueous mist onto a growth matrix, an extra-particle mycelial growth or both. The mist can contain a solute, and the solute can be one or more additives. Thus, one or more additives can be incorporated into a growth matrix and/or extra-particle mycelial growth (and thus, into the aerial mycelium obtained therefrom) via misting.

As further disclosed in US2020/0024557A1, a mycelial panel can be infused with at least one additive.

In some embodiments, one or more additives is added to a mycelium during the incubation time period. In some embodiments, one or more additives is added to a mycelium after the incubation time period. In some embodiments, one or more additives is added to a mycelium after extraction from the growth matrix.

In some embodiments, one or more additives is added during one or more post-processing steps. Thus, one or more additives can be incorporated into a mycelium by injection into a mycelium, during boiling (e.g., by incorporating additives in the aqueous solution used for boiling), during brining (e.g., in a brine fluid), during fatting (e.g., in the fat), or at any time prior to packaging. An additive can be included with the packaged goods.

An edible mycelium of the present disclosure, in any form, including an aerial mycelium for use as a food ingredient, a food product, a strip of mycelium-based bacon, and the like, can be packaged to provide a finished product. The package can include a label describing cooking instructions, storage or handling instructions, nutritional information, or a combination thereof.

In some aspects, the present disclosure provides for a method of cooking at least one edible strip of mycelium-based bacon. The method can comprise at least one of pan frying and baking. The pan frying and baking can be at a temperature within a range of about 275° F. to about 400° F. The cooking can be terminated when the edible strip of mycelium-based bacon is crisp.

### EXAMPLES

The following sets forth several non-limiting examples of making mycelia of the present disclosure and of processing mycelia of the present disclosure.

### Example 1

Growth media was prepared by hand mixing corn stover substrate (375 g) with poppy seed (90 g), maltodextrin (16 g), calcium sulfate (5 g), and water to about 65% moisture content (w/w) in polypropylene bags. The resulting growth media was pretreated by sterilization at 121° C. at 15 psi for 60 minutes, cooled to room temperature, then inoculated with Ganoderma sessile white millet grain spawn under aseptic conditions.

The resulting growth media (i.e. growth matrix) was placed in an uncovered Pyrex food dish with a volume of 59 cubic inches to a density of 26.5 pounds per cubic foot (pcf)) and incubated for a time period of 7 days in a growth chamber having an atmosphere maintained at 5% (v/v) CO₂, 14 to 20% (v/v) O₂, and >99% relative humidity via evaporative moisture, throughout the incubation time period. Growth chamber atmospheric content was maintained based on CO₂ and fresh air injection to maintain the given CO₂ setpoint, as such O₂ and other atmospheric components are maintained indirectly and fluctuate as a function of fungal respiration. Throughout the incubation period, the temperature was maintained at 85° F. The incubation was performed entirely in the dark. The growth chamber was equipped with a fan, which provided a flow of air (the air containing the same components as the growth chamber atmosphere described above) directed substantially parallel to the growth matrix at a rate within a range of about 70 to 100 linear feet per minute throughout the incubation period. The growth chamber was further equipped with a commercial ultrasonic mister that, in this case, was not operated thereby excluding mist from the growth environment.

At the end of the incubation time period, the Pyrex dish with growth matrix and resulting extra-particle mycelial growth was removed from the growth chamber, and the extra-particle mycelial growth was manually extracted from the growth matrix using a scalpel as an appressed, distinctly non-floccose and non-aerial, positively gravitropic and thigmotropic, contiguous mycelium sheet which grew along the exterior face of the Pyrex dish (11.3 g) having a moisture content of about 79% (w/w) (as determined via a Mettler Toledo HB43-S series halogen moisture analyzer), a mean thickness of 2.5 mm with a maximum thickness of 9.3 mm and a mean native density of 30 pcf. The mycelium sheet was dried at 110° F. for 24 hours to a final moisture content of equal to or less than 10% (w/w), after which the mean dry density of the mycelium sheet was 4.2 to 7.5 pcf.

### Example 2

An appressed mycelium was obtained essentially as described in Example 1, with the following exceptions: the corn stover was replaced with maple flour substrate with an approximate particle size of 0.5 mm (800 g); calcium sulfate was excluded from the growth media; the growth media was inoculated with Pleurolus ostreatus white millet grain spawn rather than with Ganoderma sessile; the Pyrex food dish was filled with growth matrix to a density of 32 pcf; and the incubation temperature was 75° F.

At the end of the incubation time period, the Pyrex dish with growth matrix and resulting extra-particle mycelial growth was removed from the growth chamber, and the extra-particle mycelial growth was manually extracted from the growth matrix using a scalpel as an appressed, distinctly non-floccose and non-aerial, felty to sub-felty, positively gravitropic and thigmotropic, contiguous mycelium sheet which grew along the exterior face of the Pyrex dish (3.8 g) having a moisture content of about 77% (w/w), a mean thickness of 2.5 mm with a maximum thickness of 8.5 mm. The mycelium sheet was desiccated at room temperature for 24 hours to a final moisture content of equal to or less than 10% (w/w).

### Example 3

Growth media was prepared by hand mixing corn stover substrate (375 g) with poppy seed (90 g), maltodextrin (16 g), calcium sulfate (5 g), and water to about 65% moisture content (w/w) in polypropylene bags. The resulting growth media was pretreated by sterilization at 121° C. at 15 psi for 60 minutes, cooled to room temperature, then inoculated with Ganoderma sessile white millet grain spawn under aseptic conditions.

For each growth replicate the resulting growth media (i.e. growth matrix) was placed in an uncovered Pyrex food dish with a volume of 59 cubic inches to a density of 26.5 pcf and incubated for a time period of 7 days in a growth chamber having an atmosphere maintained at 5% (v/v) CO₂, 14 to 20% (v/v) O₂, and >99% relative humidity via evaporative moisture, throughout the incubation time period. Growth chamber atmospheric content was maintained based on CO₂ and fresh air injection to maintain the given CO₂ setpoint, as such O₂ and other atmospheric components are maintained indirectly and fluctuate as a function of fungal respiration. Throughout the incubation period the temperature was maintained within the range of 85 to 90° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 70 to 100 linear feet per minute throughout the incubation period. The growth chamber was further equipped with a commercial ultrasonic mister supplied with tap water having a conductivity of between 400 and 500 microsiemens/cm operated at a 2% duty cycle over a 360 second cycle period. The mist was circulated within the growth chamber via the directed airflow resulting in mist deposition onto the surface of the growth matrix and the subsequent extra-particle mycelial growth at a mist deposition rate of 144 microliters/cm²/hour, and a mean mist deposition rate of 3 microliters/cm²/hour throughout the incubation time period.

At the end of the incubation time period, the Pyrex dish with growth matrix and resulting extra-particle aerial mycelial growth was removed from the growth chamber, and the extra-particle aerial mycelial growth was manually extracted from the growth matrix using a hand saw affixed with a scalloped blade as a multitude of discrete bulbose pieces of negatively gravitropic aerial mycelium (73-88 g) having a moisture content of about 91-93% (w/w), a mean thickness of >10 mm and a mean native density of 39-64 pcf. The harvested mycelium was dried at 110° F. for 24 hours to a final moisture content of equal to or less than 10% (w/w), after which the mean dry density of the panel was 4.2 to 5.4 pcf.

### Example 4

Aerial mycelium was prepared as described in Example 3, with the following exceptions. Throughout the incubation period the temperature was maintained at a temperature of 85° F. The ultrasonic mister was operated at a 0.3% duty cycle over a 1800 second cycle period. The mist was deposited onto the surface of the growth matrix and the resulting extra-particle mycelial growth at a mist deposition rate of 64 microliters/cm²/hour, and a mean mist deposition rate of 0.2 microliters/cm²/hour throughout the incubation time period.

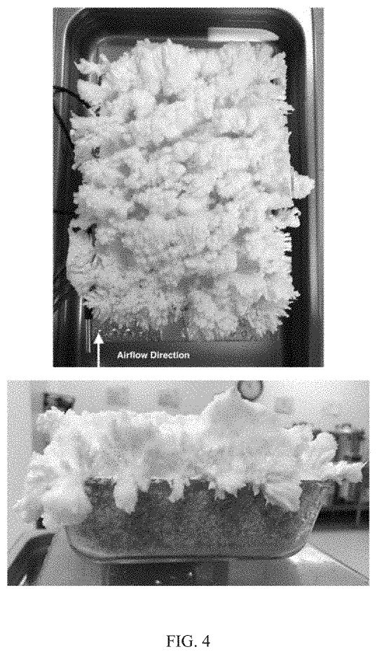

At the end of the incubation time period, the Pyrex dish with growth matrix and resulting extra-particle aerial mycelial growth was removed from the growth chamber (FIG. 4), and the extra-particle aerial mycelial growth was manually extracted from the growth matrix using a hand saw affixed with a scalloped blade as a multitude of discrete coralloid to bulbose-coralloid pieces of negatively gravitropic aerial mycelium (58 g) having a moisture content of about 89% (w/w), a mean thickness of >10 mm and a mean native density of 32 pcf. The harvested mycelium was dried at 110° F. for 24 hours to a final moisture content of equal to or less than 10% (w/w), after which the mean dry density of the panel was 4.15 pcf.

### Example 5

Aerial mycelium was prepared as described in Example 3, with the following exceptions. Throughout the incubation period the temperature was maintained at 85° F. The ultrasonic mister was operated at a 0.2% duty cycle over a 1800 second cycle period. The mist was deposited onto the surface of the growth matrix and the resulting extra-particle mycelial growth at a mist deposition rate of 18 microliters/cm²/hour, and a mean mist deposition rate of 0.03 microliters/cm²/hour throughout the incubation time period.

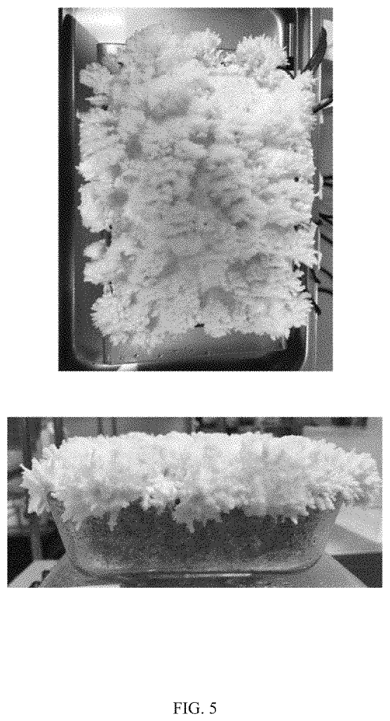

At the end of the incubation time period, the Pyrex dish with growth matrix and resulting extra-particle aerial mycelial growth was removed from the growth chamber (FIG. 5), and the extra-particle aerial mycelial growth was manually extracted from the growth matrix using a hand saw affixed with a scalloped blade as a multitude of discrete coralloid to bulbose-coralloid pieces of negatively gravitropic aerial mycelium (35-47 g) having a moisture content of about 85-86% (w/w), a mean thickness of >10 mm and a mean native density of 19-22 pcf. The harvested mycelium was dried at 110° F. for 24 hours to a final moisture content of equal to or less than 10/(w/w), after which the mean dry density of the panel was 3.4 to 3.7 pcf.

### Example 6

Aerial mycelium was prepared as described in Example 3, with the following exceptions. Throughout the incubation period the temperature was maintained at 85° F. The ultrasonic mister was placed beneath an acrylic box with a ¾″ opening from which, when the mister was in operation, mist was emitted thus reducing the mist output from the ultrasonic mister into the growth environment by >90% compared to mist emission without the acrylic box. The ultrasonic mister was operated at a 45% duty cycle over a 360 second cycle period. The mist was circulated within the growth chamber via the directed airflow resulting in mist deposition onto the surface of the growth matrix and the resulting extra-particle mycelial growth at a mist deposition rate of 0.07-0.53 microliters/cm²/hour, and a mean mist deposition rate of 0.03-0.24 microliters/cm²/hour throughout the incubation time period.

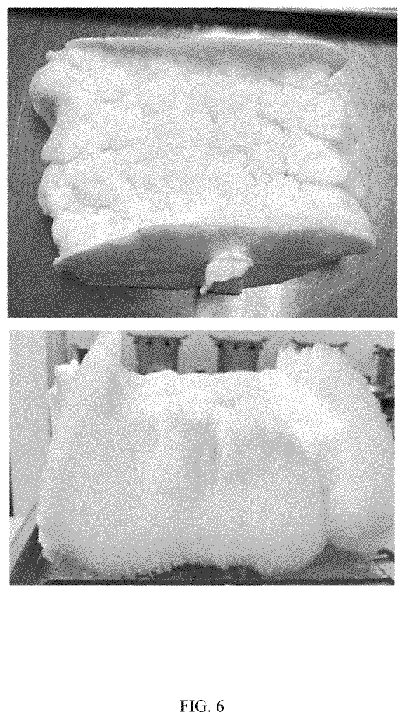

At the end of the incubation time period, the Pyrex dish with growth matrix and resulting extra-particle aerial mycelial growth was removed from the growth chamber (FIG. 6), and the extra-particle aerial mycelial growth was manually extracted from the growth matrix using a hand saw affixed with a scalloped blade as a contiguous mat of negatively gravitropic, bulbose, floccose to sub-cottony, aerial mycelium (80-122 g) having a moisture content of about 80-87% (w/w), a thickness of 19-34 mm and a native density of 4-14 pcf. The harvested mycelium mat was dried at 110° F. for 24 hours to a final moisture content of equal to or less than 10% (w/w), after which the mean dry density of the panel was 1.1 to 2.2 pcf.

### Example 7

Growth media was prepared by hand mixing maple flour substrate with an approximate particle size of 0.5 mm (800 g) with poppy seed (90 g), maltodextrin (14 g), and water to about 65% moisture content (w/w) in polypropylene bags. The resulting growth media was pretreated by sterilization at 121° C. at 15 psi for 60 minutes, cooled to room temperature, then inoculated with Pleurotus ostrealtus white millet grain spawn under aseptic conditions.

The resulting growth media (i.e. growth matrix) was placed in an uncovered Pyrex food dish with a volume of 59 cubic inches to a density of 32 pcf and incubated for a time period of 7 days in a growth chamber having an atmosphere maintained at 5% (v/v) CO₂, 14 to 20% (v/v) O₂, and >99% relative humidity via evaporative moisture, throughout the incubation time period. Growth chamber atmospheric content was maintained based on CO₂ and fresh air injection to maintain the given CO₂ setpoint, as such O₂ and other atmospheric components are maintained indirectly and fluctuate as a function of fungal respiration. Throughout the incubation period, the temperature was maintained at 75° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 70 to 100 linear feet per minute throughout the incubation period. The growth chamber was further equipped with a commercial ultrasonic mister supplied with tap water having a conductivity of between 400 and 500 microsiemens/cm. The ultrasonic mister was placed beneath an acrylic box with a ¾″ opening from which, when the mister was in operation, mist was emitted thus reducing the mist output from the ultrasonic mister into the growth environment by >90% compared to mist emission without the acrylic box. The ultrasonic mister was operated at a 45% duty cycle over a 360 second cycle period. The mist was circulated within the growth chamber via the directed airflow resulting in mist deposition onto the surface of the growth matrix and the resulting extra-particle mycelial growth at a mist deposition rate of 0.24 microliters/cm²/hour, and a mean mist deposition rate of 0.11 microliters/cm²/hour throughout the incubation time period.

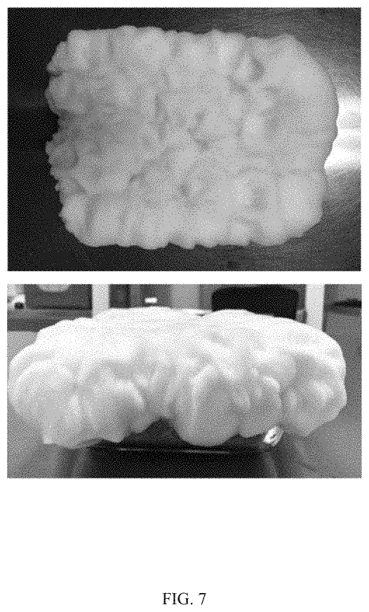

At the end of the incubation time period, the Pyrex dish with growth matrix and resulting extra-particle aerial mycelial growth was removed from the growth chamber (FIG. 7), and the extra-particle aerial mycelial growth was manually extracted from the growth matrix using a hand saw affixed with a scalloped blade as a contiguous mat of negatively gravitropic, bulbose, floccose to sub-cottony, aerial mycelium (59 g) having a moisture content of about 88% (w/w), a mean thickness of 19.8 mm and a mean native density of 10 pcf. The harvested mycelium mat was desiccated at room temperature for 24 hours to a final moisture content of equal to or less than 100% (w/w), after which the mean dry density of the panel was 1.4 pcf.

### Example 8

Mist deposition rate and mean mist deposition rate are measurable by a variety of methods. In one method, the mist deposition rate or mean mist deposition rate was measured by placing one or more open Petri dishes of known surface area in a growth environment during an incubation period for at least 24 hours throughout which mist is introduced into the growth environment, collecting the mist deposited in the open Petri dish(es), determining the total volume or mass of collected mist, and dividing the volume or mass by the period of time.

### Example 9

Hyphal filament width. Aerial and appressed mycelia were obtained essentially as described in Examples 1 to 8 and 11 to 23. After extraction from the growth matrix, the mycelium was dried for 18 hours at 110° F., after which the residual moisture content was less than about 10% (w/w) of the total mass of the mycelium. Dried aerial mycelia exhibited about 50% contraction. Sections were sliced along the thickness of the dried mycelium and embedded in epoxy resin. The epoxy embedded mycelium was then microsectioned and optically analyzed via autofluorescence to determine the hyphal width of the mycelial tissue. Alternatively, the tissue was sampled fresh via a simple tease mount, stained, and manual imaging and cell width measurement performed. The results indicated that the mean hyphal width ranged from about 0.2 micron to about 15 microns.

### Example 10

Aerial mycelium was prepared as described in Example 7, with the following exceptions. The incubation time period was 9 days. The ultrasonic mister was supplied with distilled water having a conductivity of about 3 microsiemens/cm. The mist was deposited onto the surface of the growth matrix and the resulting extra-particle mycelial growth at a mist deposition rate of 0.2 microliters/cm²/hour, and a mean mist deposition rate of 0.09 microliters/cm²/hour throughout the incubation time period.

A Wenglor OPT20 laser rangefinder was affixed to the top exterior portion of the growth chamber, where the growth chamber was made of clear acrylic, such that the laser with a spot size of 9 mm emitted at 660 nm was facing the growth matrix. The output of the laser rangefinder was integrated with the growth chamber such that the distance between the growth matrix, and subsequent aerial growth produced from the growth matrix, and the laser rangefinder during the incubation period was detected and recorded in real time during the incubation period. As such the aerial growth rate was monitored over the 9 day incubation period in order to detect when aerial growth was occurring and when aerial growth ceased indicating transition to the stationary phase, at which point the incubation period was ended.

At the end of the incubation time period, the Pyrex dish with growth matrix and resulting extra-particle aerial mycelial growth was removed from the growth chamber, and the extra-particle aerial mycelial growth was manually extracted from the growth matrix using a hand saw affixed with a scalloped blade as a contiguous mat of negatively gravitropic, bulbose, floccose to sub-cottony, aerial mycelium (63 g) having a moisture content of about 91% (w/w), a mean thickness of 20.01 mm, a maximum thickness of 30.36 mm, and a mean native density of 14 pcf. The harvested mycelium mat was desiccated at room temperature for 24 hours to a final moisture content of equal to or less than 10% (w/w), after which the mean dry density of the panel was 1.6 pcf.

### Example 11

Aerial mycelium was prepared as described in Example 7, with the following exceptions. The incubation time period was 9 days. The ultrasonic mister was supplied with distilled water having a conductivity of about 3 microsiemens/cm. The mist was deposited onto the surface of the growth matrix and the resulting extra-particle mycelial growth at a mist deposition rate of 0.35 microliters/cm²/hour, and a mean mist deposition rate of 0.16 microliters/cm²/hour throughout the incubation time period.

At the end of the incubation time period, the Pyrex dish with growth matrix and resulting extra-particle aerial mycelial growth was removed from the growth chamber, and the extra-particle aerial mycelial growth was manually extracted from the growth matrix using a hand saw affixed with a scalloped blade as a contiguous mat of negatively gravitropic, bulbose, floccose to sub-cottony, aerial mycelium (73 g) having a moisture content of about 89% (w/w), a mean thickness of 35.7 mm, a maximum thickness of 50.38 mm, and a mean native density of 10 pcf. The harvested mycelium mat was desiccated at room temperature for 24 hours to a final moisture content of equal to or less than 10% (w/w), after which the mean dry density of the panel was 1.4 pcf.

### Example 12

Growth media was prepared by combining via machine mixing on a dry mass basis maple flour substrate of an approximate particle size of 0.5 mm (87.5%) with poppy seed (10%), maltodextrin (20%) and calcium sulfate (0.5%). The mixed substrate was hydrated to about 65% moisture content (w/w) and sterilized in a mixing pressure vessel at 20 psi (130° C.) for 30 minutes. After cooling to below 26° C. the resulting growth media was inoculated with Pleurotus ostreatus white millet grain spawn under aseptic conditions.

The resulting growth media (i.e. growth matrix) was dispensed into twenty-four uncovered Cambro food pans with a volume of 560 cubic inches at a rate of 1767 g of growth media per pan and incubated for a time period of 13 days in a growth chamber having an atmosphere maintained at an average of 0.2% (v/v) CO₂, 14 to 20% (v/v) O₂, and approximately 99.8% relative humidity throughout the incubation time period. Growth chamber atmospheric content was maintained based on CO₂ and fresh air injection to maintain the given CO₂ setpoint, as such O₂ and other atmospheric components are maintained indirectly and fluctuate as a function of fungal respiration. Throughout the incubation period, the temperature was maintained between 65 and 72.5° F. The incubation was performed entirely in the dark. The growth chamber was equipped with forced air circulation, which provided a flow of air (the air containing the same components as the growth chamber atmosphere described above) directed substantially parallel to the surface of the growth matrix of each of the 24 Cambro trays, which are arranged on shelves such that there is adequate volume around each tray to allow for airflow, at a rate within a range of about 70 to 100 linear feet per minute throughout the incubation period. The growth chamber was further equipped with a commercial ultrasonic mister supplied with tap water having a conductivity of between about 400 and 500 microsiemens/cm. The ultrasonic mister was placed such that mist was emitted into the air stream, thereby disbursing mist homogeneously into the growth chamber. The ultrasonic mister was operated at a 100% duty cycle. The mist was circulated within the growth chamber via the directed airflow resulting in mist deposition onto the surface of the growth matrix of each Cambro tray and the resulting extra-particle mycelial growth at a mist deposition and a mean mist deposition rate each ranging from 0.16 to 0.68 microliters/cm²/hour (depending on Cambro tray position within the growth chamber) throughout the incubation time period.

At the end of the incubation time period, each Cambro tray containing the growth matrix and resulting extra-particle aerial mycelial growth was removed from the growth chamber, and the extra-particle aerial mycelial growth was mechanically extracted from the growth matrix as a contiguous mat of negatively gravitropic, bulbose, floccose to sub-cottony, aerial mycelium (671-766 g per tray) having a moisture content of about 91% (w/w) and a mean thickness of >10 mm.

### Example 13

The methods disclosed herein may also be performed according to the follow contemplated protocol.

Growth media is prepared by combining by machine mixing in a sterile vessel maple flour substrate (1545 g; approximate particle size 0.5 mm, pretreated by sterilization at 265° F. at 20 psi for 30 minutes) with poppy seed (180 g), maltodextrin (32 g) and calcium sulfate (10 g). The resulting growth media is then inoculated with Pleurotus ostreatus (Jacquin: Fries) strain ATCC 58753 NRRL 2366 white millet grain or Pleurotus ostreatus ATCC 56761 (180 g).

The resulting growth matrix is placed in an uncovered Cambro food pan with a volume of 560 cubic inches and incubated for a time period of 13 days in a growth chamber having a growth atmosphere maintained at 5% (v/v) CO₂, 14 to 20% (v/v) O₂, atmospheric N₂ (about 78% (v/v), and 99% relative humidity, throughout the incubation time period. Throughout the incubation period, the temperature is maintained within the range of 65 to 70° F. The incubation is performed entirely in the dark. The growth chamber is equipped with an airflow box, which provides a flow of air (the air containing the same components as the growth chamber atmosphere described above) directed substantially parallel to the surface of the growth matrix at a rate of about 81 linear feet per minute throughout the incubation period. The growth chamber is further equipped with a submersible misting puck apparatus operated at a 40% duty cycle over a 180 second cycle period, and mist is deposited onto the surface of the growth matrix and the resulting extra-particle mycelial growth at a mean mist deposition rate within a range of 0.30 to 0.35 microliters/cm²/hour throughout the incubation time period.

At the end of the incubation time period, the resulting extra-particle aerial mycelial growth is removed from the chamber and mechanically extracted from the growth matrix as a single panel of aerial mycelium. The panel (600 g) had a moisture content of about 90% (w/w), a thickness of about 30 to 60 mm and a mean density of 10 to 15 pounds per cubic foot.

### Example 14

Growth media was prepared by combining by machine mixing in a sterile vessel maple flour substrate (1545 g; approximate particle size 0.5 mm), poppy seed (180 g), maltodextrin (32 g) and calcium sulfate (10 g). The mixture was hydrated to a final moisture content of 62% (w/w), sterilized at 265° F. at 20 psi for 30 minutes, and cooled. The resulting growth media was then inoculated with fungal inoculum containing Pleurotus ostreatus spawn and white millet grain.

The resulting growth matrix was placed in an uncovered Cambro food pan with a volume of 560 cubic inches and incubated for a time period of 13 days in a growth chamber having a growth atmosphere maintained at 5% (v/v) CO₂, 14 to 20% (v/v) O₂, atmospheric N₂ (about 78% (v/v), and 99% relative humidity, throughout the incubation time period. Throughout the incubation period, the temperature was maintained within the range of 65 to 70° F. The incubation was performed entirely in the dark. The growth chamber was equipped with an airflow box, which provided a flow of air (the air containing the same components as the growth chamber atmosphere described above) directed substantially parallel to the surface of the growth matrix at a rate of about 81 linear feet per minute throughout the incubation period. The growth chamber was further equipped with a submersible misting puck apparatus operated at a 40% duty cycle over a 180 second cycle period, and mist was deposited onto the surface of the growth matrix and the resulting extra-particle mycelial growth at a mean mist deposition rate of within a range of 0.30 to 0.35 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (600 g) having a moisture content of about 90% (w/w), a thickness of about 38 to 64 mm and a mean native density of 5.5 pounds per cubic foot. The panel was dried at 110° F. for 18 hours to a final moisture content of about 10% (w/w), after which the mean dry density of the panel of 0.55 pounds per cubic foot.

### Example 15

Growth media was prepared by machine mixing, in a sterile vessel, maple flour substrate (1545 g; approximate particle size 0.5 mm) with defatted soy flour (150 g). The mixture was hydrated to a final moisture content of 60 to 65% (w/w), sterilized at 265° F. at 20 psi for 30 minutes, and cooled. The resulting growth media was then inoculated with fungal inoculum containing Pleurolus ostreatus spawn and white millet grain.

The resulting growth matrix was placed in an uncovered Cambro food pan with a volume of 560 cubic inches and incubated for a time period of 13 days in a growth chamber having a growth atmosphere of 5% (v/v) CO₂, 14 to 20% (v/v) O₂, 78% (v/v) N₂, and 99% relative humidity. Throughout the incubation period, the temperature was maintained within the range of 65 to 70° F. The incubation was performed entirely in the dark. The growth chamber was equipped with an airflow box, which provided a flow of air (the air containing the same components as the growth chamber atmosphere described above) directed substantially parallel to the surface of the growth matrix at a rate of about 81 linear feet per minute throughout the incubation period. The growth chamber was further equipped with a submersible misting puck apparatus operated at a 40% duty cycle over a 180 second cycle period, and mist was deposited onto the surface of the growth matrix and the resulting extra-particle mycelial growth at a mist deposition rate of 0.35 microliters/cm² hour, and a mean mist deposition rate of 0.30 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (1000 g) having a moisture content of about 90% (w/w), a thickness of about 38 to 75 mm and a mean native density of 8 pounds per cubic foot. The panel was dried at 110° F. for 18 hours to a final moisture content of about 10% (w/w), after which the dry density of the panel was 0.8 pounds per cubic foot.

### Example 16

Aerial mycelium was prepared as described in Example 15, with the following exceptions. The growth media was prepared by combining by aseptically hand mixing maple flour substrate (1545 g; approximate particle size 0.5 mm) with chick pea flour (150 g) prior to the hydration, sterilization, cooling and inoculation with fungal inoculum containing Pleurolus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (530 g) having a moisture content of about 90% (w/w), a thickness of about 38 to 50 mm and an estimated mean native density of 5.25 pounds per cubic foot. The panel was dried at 110° F. for 18 hours to a final moisture content of about 10% (w/w), after which the dry density of the panel was 0.53 pounds per cubic foot.

### Example 17

Aerial mycelium was prepared as described in Example 15, with the following exceptions. The growth media was prepared by combining by aseptically hand mixing maple flour substrate (1545 g; approximate particle size 0.5 mm) with millet seed flour (150 g) prior to the hydration, sterilization, cooling and inoculation with fungal inoculum containing Pleurolus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (150 g) having a moisture content of about 90% (w/w), a thickness of about 13 to 26 mm and an estimated mean native density of 3.75 pounds per cubic foot. The panel was dried at 110° F. for 18 hours to a final moisture content of about 10% (w/w), after which the dry density of the panel was 0.38 pounds per cubic foot.

### Example 18

Aerial mycelium was prepared as described in Example 15, with the following exceptions. The growth media was prepared by machine mixing in a sterile vessel maple flake substrate (1250 g; approximate particle size 2.0 mm) with defatted soy flour (150 g) prior to the hydration, sterilization, cooling and inoculation with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (500 g) having a moisture content of about 90% (w/w), a thickness of about 13 to 60 mm and a mean native density of 9.75 pounds per cubic foot. The panel was dried at 110° F. for 18 hours to a final moisture content of about 10% (w/w), after which the dry density of the panel was 0.98 pounds per cubic foot.

### Example 19

Aerial mycelium was prepared as described in Example 15, with the following exceptions. The growth media was prepared by combining by aseptically hand mixing oak flake substrate (1250 g; approximate particle size 2.0 mm) with defatted soy flour (150 g) prior to hydration, sterilization, cooling and inoculation with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (210 g) having a moisture content of about 90% (w/w), a thickness of about 7 to 38 mm and an estimated mean native density of 7.5 pounds per cubic foot. The panel was dried at 110° F. for 18 hours to a final moisture content of about 10% (w/w), after which the dry density of the panel was 0.75 pounds per cubic foot.

### Example 20

Aerial mycelium was prepared as described in Example 15, with the following exceptions. The growth media was prepared by machine mixing in a sterile vessel oak pellet substrate (680 g to 700 g; approximate particle size 2.0 to 4.0 mm) with soybean hull pellets (680 g to 700 g) prior to hydration, sterilization, cooling and inoculation with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (350 g) having a moisture content of about 90% (w/w), a thickness of about 13 to 51 mm and an estimated mean native density of 7.25 pounds per cubic foot. The panel was dried at 110° F. for 18 hours to a final moisture content of about 10% (w/w), after which the dry density of the panel was 0.73 pounds per cubic foot.

### Example 21

Aerial mycelium was prepared as described in Example 15, with the following exceptions. The growth media was prepared by combining by aseptically hand mixing maple chip substrate (1350 g; approximate particle size 50.0 mm) with defatted soy flour (150 g) prior to hydration, sterilization, cooling and inoculation with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (375 g) having a moisture content of about 90% (w/w), a thickness of about 10 to 38 mm and an estimated mean native density of 10.1 pcf. The panel was dried at 110° F. for 18 hours to a final moisture content of about 10% (w/w), after which the dry density of the panel was 0.1 pcf.

### Example 22

Aerial mycelium was prepared as described in Example 15, with the following exceptions. Growth media was prepared machine mixing in a vessel maple flake substrate (1250 g; approximate particle size 2.0 mm) with defatted soy flour (150 g). The mixture was hydrated to a final moisture content of 60 to 65% (w/w), pasteurized at 212° F. at 0-5 psi for 30 minutes, and cooled. The resulting growth media was then inoculated with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (600 g) having a moisture content of about 90% (w/w), a thickness of about 26 to 60 mm and a mean native density of 7 pcf. The panel was dried at 110° F. for 18 hours to a final moisture content of about 10% (w/w), after which the dry density of the panel was 0.7 pounds per cubic foot.

### Example 23

Aerial mycelium was prepared as described in Example 15, with the following exceptions. The growth media was prepared by combining by aseptically hand mixing vermiculite substrate (1200 g; approximate particle size 0.5 to 1.0 mm), poppy seed (180 g), maltodextrin (32 g) and calcium sulfate (10 g) prior to hydration, sterilization, cooling and inoculation with fungal inoculum containing Pleurotus ostreatus spawn and white millet grain.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium (20 g) having a moisture content of about 90% (w/w), a thickness of about 10 to 20 mm and an estimated and approximate mean native density of 0.6 pounds per cubic foot. The panel was dried at 110° F. for 18 hours to a final moisture content of about 10% (w/w), after which the dry density of the panel was 0.06 pounds per cubic foot.

### Example 24

Malt Extract Agar (MEA) was prepared by dissolving 20 g/L malt extract and 20 g/L agar agar in distilled water and autoclaving at 121° C. for 10 minutes. The MEA was cooled to 65° C. and dispensed into 90×15 mm petri dishes at rate of 20 mL per dish to an approximate depth of 5 mm allowing for a 10 mm headspace when the petri dish lid is applied. The petri dishes containing MEA (i.e. MEA plates) were inoculated with Pleurotus ostreatus ATCC 56761 by transferring a 0.5 mm diameter agar plug as provided as frozen in a cryogenic storage ampule by ATCC, and transferring the subculture of mycelial tissue to the center of the MEA plate under aseptic conditions. Inoculated plates were incubated in the dark at between 22-32° C. for a period of 7 days, during which Pleurotus ostreatus grows across the agar surface forming a circular, radial, zonate or non-zonate, floccose to cottony, subcottony or subfelty colony with a maximum colonial thickness from the agar surface of 5 mm and a total colony radius of 20 to 30 mm.

### Example 25

Malt Extract Agar (MEA) was prepared by dissolving 20 g/L malt extract and 20 g/L agar agar in distilled water and autoclaving at 121° C. for 10 minutes. The MEA was cooled to 65 C and dispensed into 90×15 mm petri dishes as at rate of 20 mL per dish to an approximate depth of 5 mm allowing for a 10 mm headspace when the petri dish lid is applied. The petri dishes containing MEA (i.e. MEA plates) were inoculated with Ganoderma sessile by either transferring a 0.5 mm diameter agar plug sub-cultured from another culture plate, or with a pre-meiotic tissue biopsy from a basidiocarp sampled under aseptic conditions, and transferring the subculture of biopsy tissue to the center of the MEA plate under aseptic conditions. Inoculated plates were incubated in the dark at between 22-32 C for a period of 7 days, during which Ganoderma sessile grows across the agar surface forming a circular, radial, zonate or non-zonate, appressed and subfelty colony with a maximum colonial thickness from the agar surface of <2 mm.

### Example 26

Malt Extract Agar (MEA) was prepared by dissolving 20 g/L malt extract and 20 g/L agar agar in distilled water and autoclaving at 121° C. for 10 minutes. The MEA was cooled to 65 C and dispensed into 90×15 mm petri dishes as at rate of 20 mL per dish to an approximate depth of 5 mm allowing for a 10 mm headspace when the petri dish lid is applied. The petri dishes containing MEA (i.e. MEA plates) were inoculated with Pleurotus ostreatus by either transferring a 0.5 mm diameter agar plug sub-cultured from another culture plate, or with a pre-meiotic tissue biopsy from a basidiocarp sampled under aseptic conditions, and transferring the subculture of biopsy tissue to the center of the MEA plate under aseptic conditions. Inoculated plates were incubated in the dark at between 22-32 C for a period of 7 days, during which Pleurotus ostreatus grows across the agar surface forming a circular, radial, zonate or non-zonate, floccose to cottony or subcottony colony with a maximum colonial thickness from the agar surface of 5 mm to 8 mm.

### Example 27

Dry white millet (800 g) was combined with distilled water (600 mL) and CaSO4 (10 g) in polypropylene bags affixed with 0.2 micron filters and pressure sterilized at 121° C. at 15 psi for 60 minutes. After cooling to room temperature, under aseptic conditions, approximately ⅙ of a 90×15 mm MEA culture of either Pleurotus ostreatus or Ganoderma sessile was cut into approximately 5×5 mm cubes and transferred into the polypropylene bag containing prepared white millet. The polypropylene bag was heat sealed and mixed by hand to distribute the MEA culture cubes through the white millet. The bag was incubated at temperatures between 22-32° C. for 7 days, mixed by hand to agitate the white millet particles, then incubated for an additional 5 to 7 days until mycelium was visible around and between the white millet particles (i.e. the white millet was colonized). The colonized white millet was then stored at 4° C. until use.

### Example 28

Kramer shear force. Kramer shear force was measured using an Instron® Universal Testing Machine, Model 3345 having a 1 kiloNewton (1 kN) load cell, in connection with a Kramer Shear cell, Catalog no. S5403 having a 2 kN capacity and equipped with five 3 mm thick blades.

A. Kramer Shear Force Testing for Pleurotus ostreatus Aerial Mycelia.

Two batches of aerial mycelium panels grown from Pleurotus ostreatus were prepared as follows. Briefly, growth media was prepared by combining via machine mixing on a dry mass basis maple flour substrate of an approximate particle size of 0.5 mm (87.5%) with poppy seed (10%), maltodextrin (2%) and calcium sulfate (0.5%). The mixed substrate was hydrated to about 65% moisture content (w/w) and sterilized in a mixing pressure vessel at 20 psi (130° C.) for 30 minutes. After cooling to below 26° C. the resulting growth media was inoculated with fungal inoculum containing Pleurotus ostreatus spawn and white millet grain. The resulting growth matrix was placed in an uncovered Cambro food pan with a volume of 560 cubic inches and incubated for a time period of 9 to 13 days in a growth chamber having a growth atmosphere maintained at 5% (v/v) CO₂, 14 to 20% (v/v) O₂, atmospheric N₂ (about 78% (v/v), and 99% relative humidity, throughout the incubation time period, during which the temperature was maintained within the range of 65 to 70° F. The incubation was performed entirely in the dark. The growth chamber was equipped with an airflow box, which provided a flow of air (the air containing the same components as the growth chamber atmosphere described above) directed substantially parallel to the surface of the growth matrix at a rate within a range of 125 to 155 linear feet per minute (first batch), or 220 to 275 linear feet per minute (second batch), throughout the incubation period. The growth chamber was further equipped with a submersible misting puck apparatus operated at a mean duty cycle 61%, and mist was deposited onto the surface of the growth matrix and the resulting extra-particle mycelial growth. In a typical experiment, the mist was deposited at a mean mist deposition rate of within a range of about 0.30 to about 0.35 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium having a moisture content of at least about 80% (w/w).

On the day of extraction from the growth matrix, four “fresh” panels of aerial mycelium, obtained as described above, were analyzed via Kramer shear cell testing. Briefly, specimens were sliced from the center (3) and the edge (3) of each panel to provide 24 specimens per panel. Each specimen was weighed, placed in the 1.75 inch by 1.75 inch Kramer shear cell, and sheared through the 1.75 inch by 1.75 inch cross-section extrusion grate, either in the dimension substantially parallel to the direction of aerial mycelial growth (“with grain”), or in the dimension substantially perpendicular to the direction of aerial mycelial growth (“against grain”). The maximum kilograms of force value was taken from the peak of the Load-Extension curve recorded from the load cell. The grams of material was taken from the mass of the sample obtained prior to being placed in the 1.75″×1.75″ Kramer shear cell. The maximum kilograms of force value was divided by the mass of the sample in grams to yield a kg/g ratio. The Kramer shear force for the aerial mycelia obtained from P. ostreatus was within a range of about 2 kg per gram of aerial mycelium to about 15 kg per gram of aerial mycelium. More particularly, the specimens obtained from the fresh panels exhibited Kramer shear force values in a range of 1.95 to 8.40 kg/g.

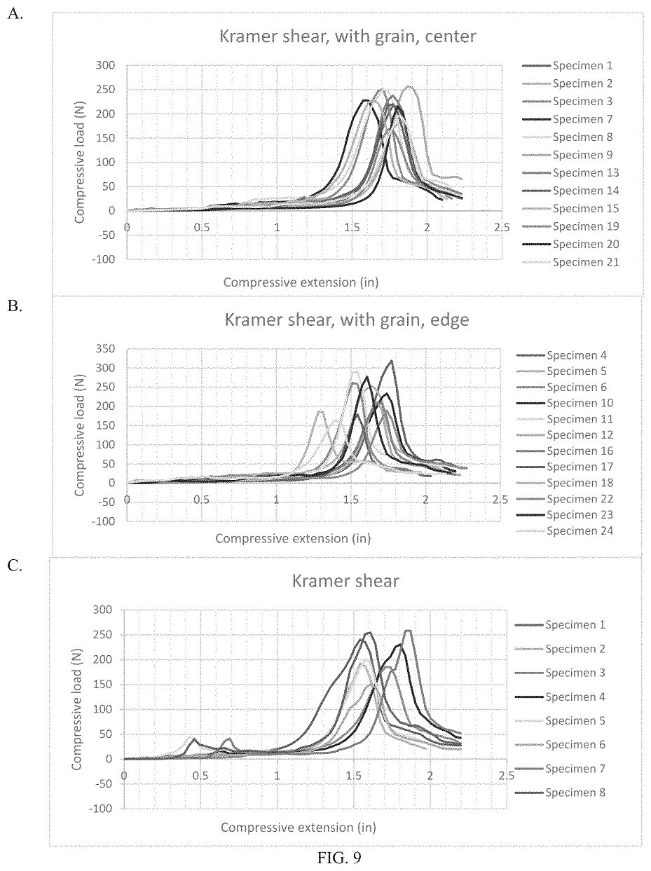

Fresh panel specimens sheared in the dimension substantially parallel to the direction of aerial mycelial growth (“with grain”; specimens 1 to 24) exhibited Kramer shear force values ranging from 1.95 to 5.04 kg/g, and a mean Kramer shear force of 2.83 kg/g. The subset of specimens cut from the center of the panel (specimens 1 to 3, 7 to 9, 13 to 15 and 19 to 21) exhibited Kramer shear force values ranging from 1.95 to 3.73 kg/g, and a mean Kramer shear force of 2.42 kg/g [FIG. 9A]. The subset of specimens cut from the edge of the panel (specimens 4 to 6, 10 to 12, 16 to 18 and 22 to 24) exhibited Kramer shear force values ranging from 2.26 to 5.04 kg/g, and a mean Kramer shear force of 3.23 kg/g [FIG. 9B]. Additional Kramer shear force test results measured on fresh panels “with grain” are described in Example 32. [See FIG. 9C.]

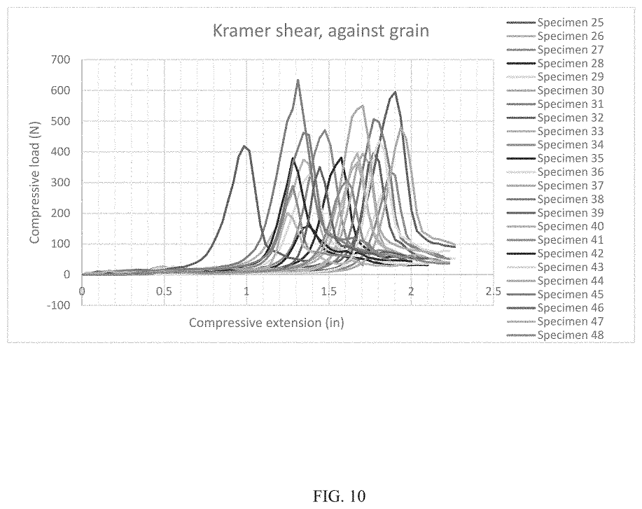

Fresh panel specimens sheared in the dimension substantially perpendicular to the direction of aerial mycelial growth (“against grain”; specimens 25 to 48), exhibited Kramer shear force values ranging from 3.04 to 8.40 kg/g, and a mean Kramer shear force of 5.85 kg/g. The subset of specimens cut from the center of the panel (specimens 25 to 27, 31 to 33, 37 to 39 and 43 to 45) exhibited Kramer shear force values ranging from 3.44 to 8.40 kg/g, and a mean Kramer shear force of 6.13 kg/g. The subset of specimens cut from the edge of the panel (specimens 28 to 38, 34 to 36, 40 to 42 and 46 to 48) exhibited Kramer shear force values ranging from 3.04 to 7.94 kg/g, and a mean Kramer shear force of 5.57 kg/g. [See FIG. 10.]

The “rise behavior” in the front half of the curves for the fresh aerial mycelia (FIG. 9 and FIG. 10) reflect the light load required to densify the material (almost “like a marshmallow”), followed by a significantly stronger load to ultimately tear (or “bite”) through it.

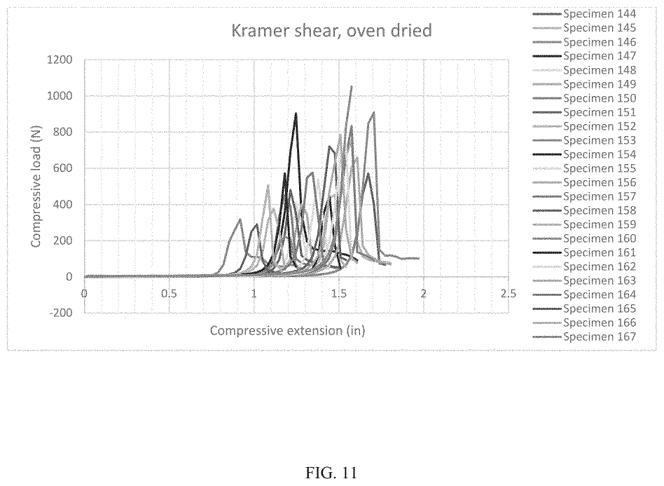

Kramer shear force values for oven-dried materials were determined as follows. Fresh panels were oven dried in an electric dryer at 110° F. for approximately 24 hours to a final moisture content of about 17%. Specimens were cut from the center and edge of the panels to provide 24 specimens. Kramer shear force testing was performed essentially as described above, shearing in the dimension substantially parallel to the direction of aerial mycelial growth (“with grain”). The specimens (specimens 144 to 167) exhibited Kramer shear force values ranging from 56.6 to 116.6 kg/g, and a mean Kramer shear force of 83.6 kg/g. [See FIG. 11.]

B. Kramer Shear Force Testing for Ganoderma sessile Aerial Mycelia.

Panels of aerial mycelium grown from Ganoderma sessile were prepared essentially as described in Example 6. At the end of the incubation time period, the resulting extra-particle aerial mycelial growth was removed from the chamber and mechanically extracted from the growth matrix as a single panel of aerial mycelium having a moisture content of at least about 80% (w/w). The panels were then allowed to acclimate to ambient atmospheric conditions (room temperature and relative humidity) for about 24 hours, but not dried in an oven or desiccated.

Aerial mycelial samples were cut from each panel and weighed, and then analyzed via the Kramer shear cell test, essentially as described for Example 28 A. Briefly, after each sample was placed in the cell, attempts were made to shear the samples through the 1.75 inch by 1.75 inch cross-section extrusion grate. These efforts overloaded the 1 kN load cell capacity, indicating that the Kramer shear force of each sample was greater than 100 kg/g of aerial mycelium.

### Example 29

Open volume (porosity). Aerial mycelia were obtained essentially as described in Example 6. After extraction from the growth matrix, the mycelium was dried for 18 hours at 110° F., after which the residual moisture content was less than about 10% (w/w) of the total mass of the mycelium. The open volume (porosity) of the dried mycelium was measured by a variety of methods.

In one experiment, sections of the aerial mycelium were sliced along its thickness and analyzed by fluid saturation. The open volume of the aerial mycelium was determined to be between 84% and 93% (v/v).

In another experiment, the aerial mycelium was embedded with a clear epoxy resin and was ground to a thin section using common thin sectioning techniques. This section was then imaged on a light microscope and the images were analyzed for open volume percentage. The open volume of the aerial mycelium was determined to be about 80% to 99% (v/v).

In yet other experiments, the aerial mycelium was inspected either by scanning electron microscopy (SEM), confocal or micro-computed tomography (CT) scanning techniques and analyzed for open volume percentage. The aerial mycelium open volume was determined to be about 80% to about 88% (v/v). Additional porosity experiments and data are disclosed in Example 39.

### Example 30

Aerial mycelium was prepared as described in Example 7, with the following exceptions. The growth media was inoculated with Pleurotus ostreatus ATCC 56761 white millet grain. The ultrasonic mister was supplied with reverse osmosis filtered water having a conductivity of between 20 and 40 microsiemens/cm.

At the end of the incubation time period, the Pyrex dish with growth matrix and resulting extra-particle aerial mycelial growth was removed from the growth chamber, and the extra-particle aerial mycelial growth was manually extracted from the growth matrix using a hand saw affixed with a scalloped blade as a contiguous mat of negatively gravitropic, bulbose, floccose to sub-cottony, aerial mycelium (63 g) having a moisture content of about 91% (w/w), a mean thickness of 20.8 mm, a maximum thickness of 36.8 mm, and a mean native density of 3.49 pcf. The harvested mycelium mat was desiccated at room temperature for 24 hours to a final moisture content of 9.4% (w/w), after which the mean dry density of the panel was 1.74 pcf.

### Example 31

Aerial mycelium was prepared as described in Example 7, with the following exceptions. The ultrasonic mister was supplied with reverse osmosis filtered water having a conductivity of between 20 and 40 microsiemens/cm.

At the end of the incubation time period, the Pyrex dish with growth matrix and resulting extra-particle aerial mycelial growth was removed from the growth chamber, and the extra-particle aerial mycelial growth was manually extracted from the growth matrix using a hand saw affixed with a scalloped blade as a contiguous mat of negatively gravitropic, bulbose, floccose to sub-cottony, aerial mycelium (63 g) having a moisture content of about 91% (w/w), a mean thickness of 22.6 mm, a maximum thickness of 36.3 mm, and a mean native density of 4.01 pcf. The harvested mycelium mat was desiccated at room temperature for 24 hours to a final moisture content of 6.9% (w/w), after which the mean dry density of the panel was 1.37 pcf.

### Example 32

A batch of 24 aerial mycelial panels was prepared as follows. To prepare each panel of the batch, growth media was prepared by machine mixing in a sterile vessel oak pellet substrate (680 g; approximate particle size 2.0 to 4.0 mm) with soybean hull pellets (680 g). The mixture was hydrated to a final moisture content of 60 to 65% (w/w), sterilized at 265° F. at 20 psi for 30 minutes, and cooled. The resulting growth media was then inoculated with fungal inoculum containing Pleurotus ostreatus spawn and white millet grain.

The resulting growth matrix was placed in an uncovered Cambro food pan with a volume of 560 cubic inches (11.5 in wide×19.5 in long×2.5 in deep) and incubated for a time period of 13 days in a growth chamber having a growth atmosphere of 5% (v/v) CO₂ and 99% relative humidity. Throughout the incubation period, the temperature was maintained within the range of 65 to 70° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 80 to 90 linear feet per minute throughout the incubation period. The growth chamber was further equipped with a submersible misting puck apparatus operated at 100% duty cycle over a 60 second cycle period. Mist was deposited onto the surface of the growth matrix and the resulting extra-particle mycelial growth at a mean mist deposition rate within a range of about 0.30 to about 0.35 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium.

Each of the 24 panels of aerial mycelium, prepared as described above, was weighed post-extraction. The maximum wet (native) mass was 1080 g, and the mean native mass was 819 g.

One of the 24 panels of aerial mycelium, prepared as described above and having been positioned near the top, center and front region of the growth chamber throughout the incubation time period, was pulled for further analysis. The panel (667 g) had a moisture content of 90.4% (w/w), a thickness of about 40 to 60 mm and an estimated mean native density of about 4.6 pounds per cubic foot. This panel, without any further processing, was sampled for physical testing as described below.

Kramer shear force. Aerial mycelial specimens (8) were sliced from the panel and then analyzed via Kramer shear cell testing. Briefly, each specimen was weighed, placed in the 1.75 inch by 1.75 inch Kramer shear cell, and sheared through the 1.75 inch by 1.75 inch cross-section extrusion grate. The maximum kilograms of force value was taken from the peak of the load-extension curve recorded from the load cell. The grams of material was taken from the specimen weight obtained prior to being placed in the 1.75″×1.75″ Kramer shear cell. The maximum kilograms of force value was divided by the mass of the specimen in grams to yield a kg/g ratio. The mean Kramer shear force for the aerial mycelial specimens (specimens 1 to 8; FIG. 9C) was 2.08+/−0.432 kg/g of material.

Tensile Strength. ASTM D638-10: Standard Test Method for Tensile Properties of Plastics was used to determine ultimate tensile strength in the dimension substantially perpendicular to the direction of aerial mycelial growth. Test samples were prepared by slicing the mycelia into 4 mm thick layers and using a CNC laser cutter to trim out testing samples having dimensions consistent with ASTM D638-10 Type IV specifications. This test was modified to accommodate wet panels, which don't cut neatly; accordingly, a rectangular block was cut, the cross-sectional area was measured (by assuming a regular width and thickness and finding the product), and the pounds per square inch at peak measured. Ultimate tensile strength was measured using on an Instron 3345 with a 5 kN load cell, and in the dimension substantially perpendicular to the direction of aerial mycelial growth (“against grain”). A single sample showed a tensile strength of 0.37 psi.

ASTM D1623 was used to determine the ultimate tensile strength in the dimension substantially parallel to the direction of mycelial growth for four samples obtained from the same panel. Ultimate tensile strength was measured using an Instron 3345 instrument with a 5 kN load cell in the dimension substantially parallel to the direction of aerial mycelial growth (“with grain”). This test as well was done with the same modification to the ASTM as described in the previous paragraph; with a larger cross-sectional area cut and assumed to be regular in geometry. A single sample showed a tensile strength of 1.1 psi.

Compression. ASTM C165-07 was used to determine the compressive properties of the samples. Specimens were cut from the center of the panel of aerial mycelium. A rectangular-prism section was measured in all three directions (width, length, height) and placed on a set of compression platens on the Instron 3345 machine (with 1 kN load cell capacity). The part was then compressed to 10° % strain, and the data over the course of the compression showed a linear relationship between stress and strain. The slope of this line (the compressive modulus) was outputted and recorded. The results of this test showed a compressive modulus at 10% strain of 0.61+/−0.02 psi and a compressive stress at 10% strain of 0.11 psi.

### Example 33

A batch of 24 aerial mycelial panels was prepared as follows. To prepare each panel in the batch, growth media was prepared by machine mixing in a sterile vessel oak pellet substrate (680 g; approximate particle size 2.0 to 4.0 mm) with soybean hull pellets (680 g). The mixture was hydrated to a final moisture content of 60 to 65% (w/w), sterilized at 265° F. at 20 psi for 30 minutes, and cooled. The resulting growth media was then inoculated with fungal inoculum containing Pleurotus ostreatus spawn and white millet grain.

The resulting growth matrix was placed in an uncovered Cambro food pan with a volume of 560 cubic inches and incubated for a time period of 13 days in a growth chamber having a growth atmosphere of 5% (v/v) CO₂ and 99% relative humidity. Throughout the incubation period, the temperature was maintained at 70° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 15 to 40 linear feet per minute throughout the incubation period. The growth chamber was further equipped with an AKIMist® Dry Fog Humidifier, which delivers a mean droplet diameter of 7 microns, and which was operated at 14.5% duty cycle over a 60 second cycle period. Mist was deposited onto the surface of the growth matrix and the resulting extra-particle mycelial growth at mean mist deposition rate within a range of about 0.3 to about 0.35 microliters/cm²/hour throughout the incubation time period.

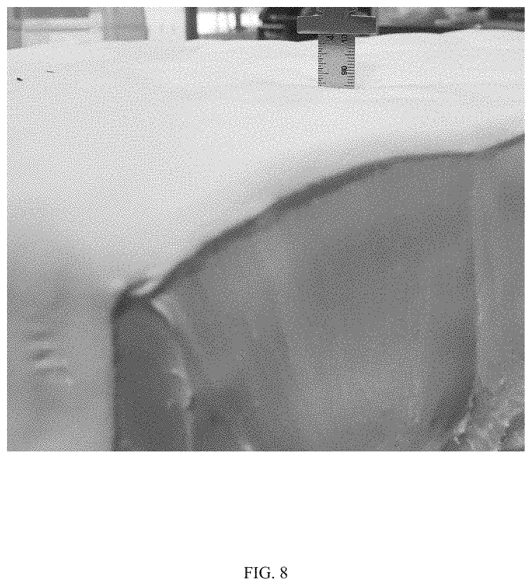

At the end of the incubation time period, the food pan containing the growth matrix and the resulting extra-particle aerial mycelial growth was removed from the growth chamber. Prior to extraction, a metal ruler was inserted into a panel (but not into the growth matrix beneath the panel) to measure the panel thickness, which was about 84 mm (FIG. 8). Additional panels in the batch were similarly measured, revealing a panel thickness within a range of about 63 to about 84 mm across the batch of aerial mycelia.

### Example 34

Five (5) batches of aerial mycelia were prepared as described below, from which nine (9) panels of aerial mycelia were analyzed for their nutritional content.

Growth media was prepared by machine mixing in a sterile vessel maple flake substrate (1250 g; approximate particle size 2.0 mm) with defatted soy flour (150 g), Batch 1, maple flour substrate (1545 g; approximate particle size 0.5 mm), poppy seed (180 g), maltodextrin (32 g) and calcium sulfate (10 g), Batches 2 and 5; or oak pellet substrate (680 g; approximate particle size 2.0 to 4.0 mm) with soybean hull pellets (680 g), Batches 3 and 4. Each mixture was hydrated to a final moisture content of 60 to 65% (w/w), pasteurized at 212° F. at 0-5 psi for 30 minutes or sterilized at 265° F. at 20 psi for 30 minutes, and cooled. The resulting growth media was then inoculated with fungal inoculum containing Pleurotus ostreatus spawn and grain.

The resulting growth matrix was placed in an uncovered Cambro food pan with a volume of 560 cubic inches and incubated for a time period of 9 to 13 days in a growth chamber having a growth atmosphere of 5% (v/v) CO₂ and 99% relative humidity. Throughout the incubation period, the temperature was maintained within the range of 65 to 70° F. The incubation was performed entirely in the dark. The growth chamber was equipped with an airflow box or a fan, which provided a flow of air directed substantially parallel to the surface of the growth matrix throughout the incubation period. The growth chamber was further equipped with a misting apparatus, which was operated at a 100% duty cycle, with the directed flow of air provided at a rate within a range of about 80 to 90 linear feet per minute (Batches 1, 2 and 3); operated at a 43% duty cycle, with the directed flow of air provided at a rate within a range of about 15 to 40 linear feet per minute (Batch 4); or operated at a rate of 61% duty cycle, with the directed flow of air provided at a rate within a range of about 125 to 275 linear feet per minute (Batch 5). Mist was deposited onto the surface of the growth matrix and the resulting extra-particle mycelial growth at a mean mist deposition rate of about 0.3 to about 0.35 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium.

Aerial mycelial panels prepared as described above (two panels from each of Batches 1, 3, 4 and 5, and one panel from Batch 2) were weighed post-extraction and further analyzed for nutritional and inorganics content according to the methods described below. The 24 panels from Batch 4 had wet (native) masses within a range of 710 g to 1044 g, with a mean mass of 894 g.

Nutritional content of aerial mycelia. All nutritional parameters were first determined on a wet (native) weight basis and then converted to a dry weight basis according to the following equation:

[100*(Wet weight basis/(100−moisture content)]=Dry weight basis; wherein the moisture content includes volatiles and is determined according to Example 34A.

A. Moisture content (including volatiles) of mycelia was determined using the Official Method of Analysis (AOAC) 925.09. Briefly, a native (undried) mycelial sample is weighed and placed into a 100° C. vacuum oven for a specific amount of time, based on sample matrix. After drying, the sample is removed from the oven and cooled in a desiccator. When cool, the weight of the dried sample is determined. The moisture content (including volatiles) is the difference between the weight of the undried sample and the weight of the sample after drying.

B. Protein content. Protein content of aerial mycelia was determined using reference methods AOAC 990.03 and AOAC 992.15. Briefly, a sample of aerial mycelium is placed into a protein analyzer combustion chamber. Following combustion, the resulting gas is analyzed for nitrogen content. Crude protein is calculated by multiplying the nitrogen content by a protein conversion factor. The standard protein conversion factor is 6.25, however, as mushrooms contain a significant amount of non-protein nitrogen as chitin, a conversion factor of 4.38 is used. [See: Organization for Economic Co-operation and Development (OECD) Environment, Health and Safety Publications Series on the Safety of Novel Foods and Feeds, No. 26, Consensus Document on Compositional Considerations for New Varieties of OYSTER MUSHROOM [Pleurotus ostreatus]: Key Food and Feed Nutrients, Anti-nutrients and Toxicants; Paris 2013; the entire content of which is hereby incorporated by reference in its entirety.]

C. Fat content. Total fat content for aerial mycelia is reported based on total triglycerides, as determined using reference method AOAC 996.06 mod. Briefly, a fat extraction method is performed. Sample or extracted fat from sample is reacted with boron-trifluoride/methanol reagent to convert fatty acids present in any form into their corresponding methyl ester forms, which are then extracted into hexanes and injected onto a capillary column gas chromatograph. Standards of known composition are used to identify the fatty acids present, and the percentage of each fatty acid as a part of the entire sample is calculated.

D.) Dietary Fiber Dietary fiber content for aerial mycelia was determined using reference method AOAC 991.43. Briefly, fat and sugar are extracted from the sample, then dried samples undergo enzymatic digestion to remove starch and protein, leaving dietary fiber.

E. Carbohydrates Carbohydrate content of aerial mycelia was calculated using the standard CFR 21 calculation. [See: 21CFR101.9. Code of Federal Regulations, Title 21, Volume 2, Revised Apr. 1, 2019; the entire content of which is hereby incorporated by reference in its entirety.] As such, carbohydrates are calculated as follows:

Total carbohydrates=[100−(crude protein+total fat+total moisture (including volatiles)+ash)].

F. Ash. Ash content, also referred to herein as “inorganic content,” is determined using reference method AOAC 942.05. Briefly, unaltered fresh sample is weighed and placed into a temperature-controlled furnace. Set temperature is maintained for a specified amount of time, typically 600° C. for 2 hours. Dried sample is transferred to a desiccator, cooled, and weighed immediately.

G. Potassium. Potassium content of aerial mycelia was determined using reference AOAC methods 984.27 mod, 927.02 mod, 985.01 mod, 965.17 mod. Briefly, samples are digested, and the resultant digest is analyzed by Inductively Coupled Plasma Optical Emission Spectrophotometry against a set of ISO certified standards.

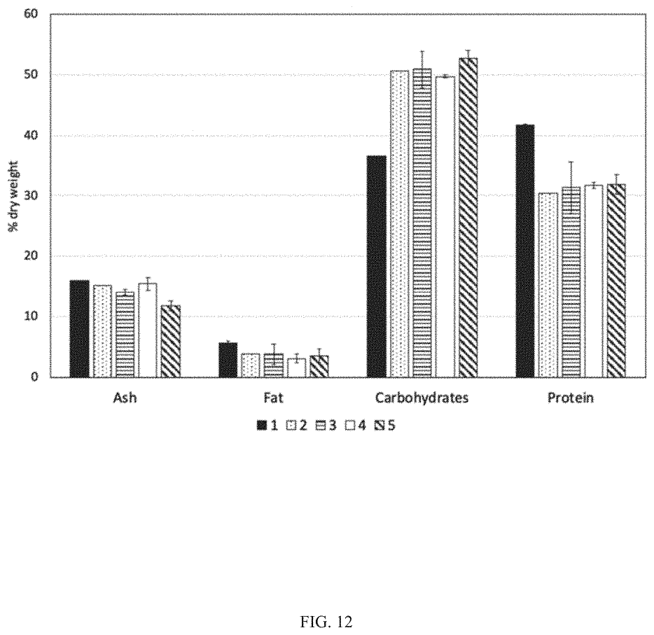

Results. The mean protein content ranged from 30.38% to 41.67% (w/w); the mean fat content ranged from 3.10% to 5.74% (w/w); the mean ash content ranged from 11.76% to 16.04% (w/w); and the mean carbohydrate content ranged from 36.48% to 52.79% (w/w); wherein each percentage is reported on a dry weight basis, and wherein each mean value is an average obtained for the two representative panels from each of Batches 1, 3, 4 and 5, or a single value for the Batch 2 panel (FIG. 12). The mean dietary fiber content ranged from 17.5% (w/w) to 31.9% (w/w); wherein each percentage is reported on a dry weight basis, and wherein each mean value is an average obtained for the two representative panels from each of Batches 1, 4 and 5, or a single value for each of Batches 2 and 3. The potassium content ranged from 4883 to 6044 mg potassium per 100 g of dry aerial mycelium.

### Example 35

Heavy metals. Heavy metal content of aerial mycelia was determined using reference methods from the Journal of AOAC Int'l 94(4): 1240-1252, and AOAC 993.1; the entire content of which is hereby incorporated by reference in its entirety Briefly, samples of aerial mycelia are digested with nitric acid in an open- or closed-vessel microwave digestion system. Analysis is performed using an Inductively Coupled Plasma with Mass Spectrometric detection. The digested samples are compared to standards of known concentration.

Panels prepared according to Example 34 were analyzed as described above and showed less than 100 ppb lead, less than 50 ppb arsenic, less than 200 ppb cadmium and less than 500 ppb mercury.

### Example 36

Five aerial mycelial panels were obtained as follows. Growth media was prepared by hand mixing maple flour substrate with an approximate particle size of 0.5 mm (800 g) with poppy seed (90 g), maltodextrin (14 g), and water to about 65% moisture content (w/w) in polypropylene bags. The resulting growth media was pretreated by sterilization at 121° C. at 15 psi for 60 minutes, cooled to room temperature, then inoculated with Pleurotus ostreatus white millet grain spawn under aseptic conditions.

The resulting growth media (i.e. growth matrix) was placed in an uncovered Pyrex food dish with a volume of 59 cubic inches to a density of 32 pcf and incubated for a time period of 7 days in a growth chamber having an atmosphere maintained at >99% relative humidity via evaporative moisture, and at a CO₂ setpoint of either 5% (v/v) CO₂ (three (3) control panels) or 0.1% (v/v) CO₂ (two (2) test panels), throughout the incubation time period. More particularly, for each control panel, growth chamber atmospheric content was maintained at the 5% (v/v) CO₂ setpoint via CO₂ and fresh air injection; as such, O₂ and other atmospheric components were maintained indirectly and fluctuated as a function of fungal respiration. For the first test panel, CO₂ and fresh air injection and increased ventilation were employed to maintain the target 0.1% (v/v) CO₂ level; the observed CO₂ level was less than 0.2% (v/v) (mean 0.04% (v/v) (400 ppm)) over the course of the incubation period. For the second test panel, progressive metabolic accumulation was allowed to occur during growth, and the CO₂ level reached a maximum of 3% (v/v) (mean 2% (v/v)) over the course of the incubation period.

Throughout the incubation period, the temperature was maintained at 75° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 70 to 100 linear feet per minute throughout the incubation period. The growth chamber was further equipped with a commercial ultrasonic mister supplied with reverse osmosis filtered water having a conductivity of between 20 and 40 microsiemens/cm. The ultrasonic mister was placed beneath an acrylic box with a ¾″ opening from which, when the mister was in operation, mist was emitted. The ultrasonic mister was operated at a 45% duty cycle over a 360 second cycle period. The mist was circulated within the growth chamber via the directed airflow resulting in mist deposition onto the surface of the growth matrix and the resulting extra-particle mycelial growth at a mist deposition rate of 0.59 microliters/cm²/hour, and a mean mist deposition rate of 0.26 microliters/cm²/hour throughout the incubation time period.

At the end of the incubation time period, each Pyrex dish with growth matrix and resulting extra-particle aerial mycelial growth was removed from the growth chamber, and the extra-particle aerial mycelial growth was manually extracted from the growth matrix using a hand saw affixed with a scalloped blade. Each panel presented as a contiguous mat of negatively gravitropic, bulbose, floccose to sub-cottony, aerial mycelium with no visible stipe, cap or spores. Yield, mean thickness (within a range of 13 to 23 mm), dry density (1 to 2 pcf) and morphology was consistent between the three positive controls and the two test panels.

### Example 37

Four aerial mycelial panels were obtained as follows. Growth media was prepared by hand mixing maple flour substrate with an approximate particle size of 0.5 mm (800 g) with poppy seed (90 g), maltodextrin (14 g), and water to about 65% moisture content (w/w) in polypropylene bags. The resulting growth media was pretreated by sterilization at 121° C. at 15 psi for 60 minutes, cooled to room temperature, then inoculated with Pleurolus ostreatus white millet grain spawn under aseptic conditions.

The resulting growth media (i.e. growth matrix) was placed in an uncovered Pyrex food dish with a volume of 59 cubic inches to a density of 32 pcf and incubated for a time period of 7 days in a growth chamber having an atmosphere maintained at >99% relative humidity via evaporative moisture and at a CO₂ setpoint of 5% (v/v) CO₂, and a temperature maintained at 75° F., throughout the incubation time period. The growth chamber was equipped with a fan, which provided a flow of air (the air containing the same components as the growth chamber atmosphere described above) directed substantially parallel to the surface of the growth matrix at a rate within a range of about 70 to 100 linear feet per minute throughout the incubation period. The growth chamber was further equipped with a commercial ultrasonic mister supplied with reverse osmosis filtered water having a conductivity of between 20 and 40 microsiemens/cm. The ultrasonic mister was placed beneath an acrylic box with a ¾″ opening from which, when the mister was in operation, mist was emitted. The ultrasonic mister was operated at a 45% duty cycle over a 360 second cycle period. The mist was circulated within the growth chamber via the directed airflow resulting in mist deposition onto the surface of the growth matrix and the resulting extra-particle mycelial growth at a mist deposition rate of 0.59 microliters/cm²/hour, and a mean mist deposition rate of 0.26 microliters/cm²/hour throughout the incubation time period.

The growth chamber was further equipped with white LED strip lights. For three (3) control panels, the incubation was performed in the dark throughout the incubation time period; for one (1) test panel, the incubation was performed with white light exposure via the LED strip light throughout the incubation time period.

At the end of the incubation time period, each Pyrex dish with growth matrix and resulting extra-particle aerial mycelial growth was removed from the growth chamber, and the extra-particle aerial mycelial growth was manually extracted from the growth matrix using a hand saw affixed with a scalloped blade. Each panel presented as a contiguous mat of negatively gravitropic, bulbose, floccose to sub-cottony, aerial mycelium. Neither the control aerial mycelial panels (grown in the dark) nor the test aerial mycelial panel (grown with exposure to white light, which includes light in the red, blue and green spectral ranges) showed any visible stipe, cap or spores. Thus, while exposing fungi to white light (and especially blue light) has been associated with the induction of fruiting and the enhancement of production efficiency of oyster mushrooms (Roshita & Goh, AIP Conference Proceedings 2030, 020110 (2018)), no fruiting bodies were observed on the test aerial mycelial panel. Yield, mean thickness (within a range of 12.5 to 23 mm), dry density (1 to 2 pcf) and morphology was consistent between the three positive controls and the one test panel.

### Example 38

Four aerial mycelia and one appressed mycelium were obtained as described follows. Growth media was prepared by hand mixing maple flour substrate with an approximate particle size of 0.5 mm (800 g) with poppy seed (90 g), maltodextrin (14 g), and water to about 65% moisture content (w/w) in polypropylene bags. The resulting growth media was pretreated by sterilization at 121° C. at 15 psi for 60 minutes, cooled to room temperature, then inoculated with Pleurotus ostreatus white millet grain spawn under aseptic conditions.

The resulting growth media (i.e. growth matrix) was placed in an uncovered Pyrex food dish with a volume of 59 cubic inches to a density of 32 pcf and incubated for a time period of 7 days in a growth chamber having an atmosphere maintained at >99% relative humidity via evaporative moisture and at a CO₂ setpoint of 5% (v/v) CO₂, and a temperature maintained at 75° F., throughout the incubation time period. The incubation was performed in the dark throughout the incubation time period. The growth chamber was equipped with a fan, which provided a flow of air (the air containing the same components as the growth chamber atmosphere described above) directed substantially parallel to the surface of the growth matrix at a rate within a range of about 70 to 100 linear feet per minute throughout the incubation period.

The growth chamber was further equipped with a commercial ultrasonic mister supplied with reverse osmosis filtered water having a conductivity of between 20 and 40 microsiemens/cm. The ultrasonic mister was placed beneath an acrylic box with a ¾″ opening from which, when the mister was in operation, mist was emitted. For three (3) control samples, the mister was operated at a 45% duty cycle over a 360 second cycle period throughout the entire incubation time period. For a first test sample, the mister was not operated (0% duty cycle) during days 1 to 3 of the incubation time period, and was subsequently operated at a 45% duty cycle over a 360 second cycle period throughout the remainder of the incubation time period. For a second test sample, the mister was not operated (0% duty cycle) at any time during the incubation time period. When in operation, the ultrasonic mister was used to circulate mist within the growth chamber via the directed airflow resulting in mist deposition onto the surface of the growth matrix and the resulting extra-particle mycelial growth at a mist deposition rate of 0.59 microliters/cm²/hour, and a mean mist deposition rate of 0.26 microliters/cm²/hour.

At the end of the incubation time period, each Pyrex dish with growth matrix and resulting mycelial growth was removed from the growth chamber, and the mycelial growth was manually extracted from the growth matrix using a hand saw affixed with a scalloped blade. Each control sample (obtained with mist deposition throughout the incubation time period) and the first test sample (obtained with mist drop-out during days 1 to 3 only) presented as a contiguous mat of negatively gravitropic, bulbose, floccose to sub-cottony, aerial mycelium. Yield, mean thickness (within a range of 13 to 23 mm), dry density (1 to 2 pcf) and morphology was consistent between the three positive controls and the first test sample. In contrast, the second test sample (obtained without mist deposition) presented as an appressed mycelium having a mean thickness of 2.5 mm.

For the positive controls, a laser rangefinder was used to measure vertical expansion kinetics of mycelia over the course of the incubation time period. Overall, the kinetic characteristics captured were exceptionally consistent between replicate growth cycles, including a flat region representing the primary myceliation phase, and a linear vertical region representing a vertical expansion phase. Calculated velocities were also highly consistent between cycles with differences in time of inflection and area under the curve of the linear region fitting rationally with yield. The primary myceliation phase included days 1 to 3 of the incubation time period. Thus, misting throughout the vertical expansion phase was sufficient to produce aerial mycelium having substantially similar characteristics to aerial mycelia obtained by depositing mist throughout the entire incubation period.

### Example 39

A batch of 6 aerial mycelial panels was prepared as follows. To prepare each panel in the batch, growth media was prepared by machine mixing in a sterile vessel oak pellet substrate (680 g; approximate particle size 2.0 to 4.0 mm) with soybean hull pellets (680 g). The mixture was hydrated to a final moisture content of 60 to 65% (w/w), sterilized at 265° F. at 20 psi for 30 minutes, and cooled. The resulting growth media was then inoculated with fungal inoculum containing Pleurotus ostreatus spawn and white millet grain.

The resulting growth matrix was placed in an uncovered Cambro food pan with a volume of 560 cubic inches and incubated for a time period of 13 days in a growth chamber having a growth atmosphere of 5% (v/v) CO₂ and 99.9% relative humidity. Throughout the incubation period, the temperature was maintained at 70° F. The incubation was performed entirely in the dark. The growth chamber was equipped with a fan, which provided a flow of air (the air containing the same components as the growth chamber atmosphere described above) directed substantially parallel to the surface of the growth matrix at a rate within a range of about 15 to 40 linear feet per minute throughout the incubation period. The growth chamber was further equipped with an AKIMist® Dry Fog Humidifier, which delivers a mean droplet diameter of 7 microns, and which was operated at 14.5% duty cycle over a 60 second cycle period. Mist was deposited onto the surface of the growth matrix and the resulting extra-particle mycelial growth at mean mist deposition rate within a range of about 0.3 to about 0.35 microliters/cm²/hour throughout the incubation time period.

At the end of the incubation time period, the food pan containing the growth matrix and the resulting extra-particle aerial mycelial growth was removed from the growth chamber, and the extra-particle aerial mycelial growth was mechanically extracted from the growth matrix as a single panel of aerial mycelium.

The six panels of aerial mycelium prepared as described above, identified as panels A, D, G, J, P and S, were weighed post-extraction and analyzed for physical properties. The six panels of aerial mycelium had native masses within a range of 706 to 810 g (mean 743 g), native volumes within a range of 0.31 to 0.34 cubic feet (mean 0.32 cubic feet), native moisture contents of about ˜90% (w/w), and native densities within a range of 4.7 pcf to 5.6 pcf (mean 5.1 pcf).

The aerial mycelial panels were then dried at 110° F. to a final moisture content of less than 10% (w/w). The dry densities were within the range of 0.47 pcf to 0.56 pcf (mean 0.51 pcf; n=6), as calculated based on the mass of the dried mycelium over the measured volume of the mycelium prior to drying, and within the range of 0.93 pcf to 1.09 pcf (mean 1.01 pcf; n=6), as calculated based on the mass of the dried mycelium over the measured volume of the mycelium after drying. Dried mycelia exhibited about 50% contraction. The skeletal density of the dried mycelium as determined via helium pycnometry was within the range of 11.7 to 23.2 pcf (mean 17.9 pcf; n=3). The percent porosity and median pore diameter of the dried mycelium as determined via liquid extrusion porosimetry was within the range of 62.2% to 78.2% (n=3) and 24.5 micron to 31.2 micron (n=3), respectively.

The thickness of each panel is reported in Table 1, including the thickness of the first and third quartiles and the mean, median and maximum thickness over the entire volume of each panel.

TABLE 1
Mean and median thickness of each aerial mycelial panel.

| Panel thickness | Panel A | Panel D | Panel G | Panel J | Panel P | Panel S |
|---|---|---|---|---|---|---|
| First quartile (mm) | 53.78 | 51.76 | 53.75 | 56.27 | 48.77 | 52.12 |
| Mean (mm) | 60.00 | 58.94 | 60.01 | 61.37 | 58.73 | 59.96 |
| Median (mm) | 65.97 | 65.33 | 65.99 | 65.60 | 66.73 | 67.80 |
| Third quartile (mm) | 69.31 | 69.09 | 69.33 | 70.03 | 70.36 | 70.55 |
| Maximum (mm) | 77.15 | 77.89 | 77.17 | 77.12 | 82.65 | 77.93 |

Thus, each panel in the batch had a thickness of at least 48 mm over 75% of the panel volume, a thickness of at least 65 mm over 50% of the panel volume, a thickness of at least 69 mm over 25% of the panel volume, a maximum thickness of at least 77 mm, and a mean thickness of at least 58 mm. Moreover, 100% of the panels in the batch met these same criteria.

Twelve (12) aerial mycelial specimens were cut from each of panels A, G and J, with six specimens cut from the edge of each panel (three of insufficient quantity for further analysis), and six specimens cut from the center of each panel (about 5 inches from the panel edge). The resulting 33 specimens were split into two groups of 15 and 18 specimens each.

Compressive modulus with compression to 10% strain. The first group of 15 specimens was analyzed using method ASTM C165-07, essentially as described in Example 32. Briefly, eight specimens were analyzed by applying compressive force (load) in the direction parallel to the direction of mycelial growth; these specimens showed a mean compressive modulus at 10% strain of 1.48±0.77 psi, and a compressive stress at 10% strain of 0.15±0.06 psi. Seven specimens were analyzed by applying compressive force (load) in the direction perpendicular to the direction of mycelial growth; these specimens showed a mean compressive modulus at 10% strain of 0.33±0.17 psi and a compressive stress at 10% strain of 0.05±0.02 psi. When taking all specimens (center and edge-cut) into consideration, the results of compressive testing in the parallel and perpendicular directions showed a mean compressive modulus at 10% strain of 0.95±0.82 psi and a compressive stress at 10% strain of 0.11±0.07 psi.

When the edge-cut specimens were analyzed by applying compressive force (load) in the direction parallel to the direction of mycelial growth, these specimens showed a mean compressive modulus at 10% strain of 0.86±0.20 psi and a compressive stress at 10% strain of 0.10±0.02 psi. When the edge-cut specimens were analyzed by applying compressive force (load) in the direction perpendicular to the direction of mycelial growth, these specimens showed a mean compressive modulus at 10% strain of 0.30±0.03 psi and a compressive stress at 10% strain of 0.049±0.004 psi.

The results for center-cut specimens are shown in Tables 2 and 3 for parallel and perpendicular compression, respectively.

TABLE 2
Compressive testing to 10% strain with compression parallel
to the direction of growth for center-cut specimens.

| Panel-specimen | Compressive modulus at | Compressive stress at |
|---|---|---|
| number-cut | 10% strain, PSI | 10% strain, PSI |
| A-5-Center | 2.04 | 0.20 |
| G-4-Center | 1.16 | 0.12 |
| G-6-Center | 1.43 | 0.14 |
| J-1-Center | 3.08 | 0.27 |
| J-3-Center | 1.56 | 0.16 |
| mean | 1.85 | 0.18 |
| standard deviation | 0.76 | 0.06 |

TABLE 3
Compressive testing to 10% strain with compression perpendicular
to the direction of growth for center-cut specimens.

| Panel-specimen | Compressive modulus at | Compressive stress at |
|---|---|---|
| number-cut | 10% strain, PSI | 10% strain, PSI |
| A-4-Center | 0.69 | 0.09 |
| A-6-Center | 0.21 | 0.04 |
| G-5-Center | 0.17 | 0.04 |
| J-2-Center | 0.34 | 0.06 |
| mean | 0.35 | 0.06 |
| standard deviation | 0.23 | 0.02 |

For center-cut specimens compressed to 10% strain, the mean compressive modulus-upon compression in the direction parallel to the direction of mycelial growth was over 5-fold greater than the mean compressive modulus upon compression in the direction perpendicular to mycelial growth; and the mean compressive stress upon compression in the direction parallel to the direction of mycelial growth was 3-fold greater than the mean compressive stress upon compression in the dimension perpendicular to mycelial growth.

Compression to 80% strain. The second set of 18 specimens was analyzed by a modified method, as follows. A rectangular-prism section was measured in all three directions (width, length, height) and placed within a rigid high-density polyethylene (HDPE) lower platen on an Instron 3345 machine (with 1 kN load cell capacity). The upper platen was affixed to the screw attenuated actuator having a dual clevis joint to enable self-alignment within the lower platen. The specimen was preloaded with 0.5 lbF which initiated the test. The specimen was then compressed to 80% strain, measured by extension, and the data over the course of the compression was plotted to provide a relationship between stress and strain (extension) and load and strain (extension). Compressive stress to about 65% strain were further extrapolated from the data.

For all specimens (cut from edge and center of panels), the compressive stress at 65% strain, upon compression in the direction perpendicular to mycelial growth, was 0.12 psi±0.08 psi. For edge specimens, the compressive stress at 65% strain, upon compression in the direction perpendicular to mycelial growth, was 0.14 psi±0.10 psi; for center specimens, the compressive stress at 65% strain, in the direction perpendicular to mycelial growth, was 0.10±0.04 psi.

Compressive stress at 80% strain for edge and center-cut samples tested by compression in the direction parallel and perpendicular to mycelial growth showed the following results: panel A: mean 7.7 psi±15 psi; panel G: mean 1.7 psi±1.8 psi; panel J: mean 3.1 psi±3.4 psi)

### Example 40

1. Mycelial tissue is cut parallel to the grain into 0.25 to 1-inch strips.
2. Cut strips are compressed to 15-75% original height, antiparallel to the grain.
3. Compressed strips are then needle-punched, to disrupt tissue network.
4. Tenderized strips are then boiled for 5 minutes in a salt brine to impart flavor and modify texture.
5. Boiled strips are then baked or pan-fried in oil at 275 to 400° F. until crispy.

### Example 41

1. Mycelial tissue is compressed to disrupt fiber alignment.
2. 0.75 to 1.25-inch strips are then cut from the compressed tissue, parallel to the grain.
3. Compressed strips are then needle-punched to disrupt tissue network.
4. Tenderized strips are then boiled for 5 minutes in a salt brine to impart flavor and modify texture.
5. Boiled strips are then pan-fried in oil at 275 to 400° F. until crispy.

### Example 42

1. Mycelial tissue is cut parallel to the grain into 0.25 to 1-inch strips.
2. Cut strips are compressed to 15-75% original height, antiparallel to the grain.
3. Compressed strips are then needle-injected, to disrupt tissue network, and the tissue matrix is injected with brine, fats, flavors, proteins, or the like.
4. Tenderized and injected strips are then cooked at 275 to 400° F. until crispy.

### Example 43

1. Mycelial tissue is cut parallel to the grain into 0.25 to 1-inch strips.
2. Cut strips are compressed to 15-75% original height, antiparallel to the grain.
3. Compressed strips are then stacked and needle-punched, to disrupt tissue network, and entangle multiple strips into one contiguous unit of material.
4. Tenderized strips are then boiled for 5 minutes in a salt brine to impart flavor and modify texture.
5. Boiled strips are then cooked at 275 to 400° F. until crispy.

### Example 44

1. Mycelial tissue is cut parallel to the grain into 0.25 to 1-inch strips.
2. Cut strips are compressed to 15-75% original height, antiparallel to the grain.
3. Compressed strips are then stacked and needle-punched, where the needle punching, density, intensity, and shape, is varied across the matrix to disrupt tissue network, and create sections that cook at different rates than others, modifying finished texture.
4. Tenderized strips are then boiled for 5 minutes in a salt brine to impart flavor, and modify texture.
5. Boiled strips are then cooked at 275 to 400° F. until crispy.

Some nonlimiting embodiments of the disclosure are listed below.

A1. A method of making an edible aerial mycelium, comprising: providing a growth matrix comprising a substrate and a fungal inoculum, wherein the fungal inoculum comprises a fungus; incubating the growth matrix as a solid-state culture in a growth environment for an incubation time period; and introducing aqueous mist into the growth environment throughout the incubation time period, or a portion thereof, wherein the aqueous mist has a mist deposition rate and a mean mist deposition rate, and the mean mist deposition rate is less than or equal to about 10 microliter/cm²/hour; thereby producing extra-particle aerial mycelial growth from the growth matrix.

A2. The method of embodiment A1, wherein: the growth environment comprises a growth atmosphere having a relative humidity, an oxygen (O₂) level and a carbon dioxide (CO₂) level, wherein the CO₂ level is at least about 0.02% (v/v) and less than about 8% (v/v); the mist deposition rate is less than or equal to about 150 microliter/cm²/hour; and the mean mist deposition rate is less than or equal to about 5 microliter/cm²/hour, or less than or equal to about 3 microliter/cm²/hour.

A3. The method of embodiment A1 or A2, further comprising removing the extra-particle aerial mycelial growth from the growth matrix, thereby providing an aerial mycelium

A4. The method of embodiment A1, A2 or A3, wherein:

(a) the carbon dioxide level is within a range of about 0.2% to about 7% (v/v), and the aerial mycelium does not contain a visible fruiting body; or
(b) the carbon dioxide level is within a range of about 0.02% to about 7% (v/v), and

(i) the incubation time period ends no later than when a visible fruiting body forms;

(ii) the incubation time period ends when a visible fruiting body forms; or

(iii) the aerial mycelium does not contain a visible fruiting body.

A5. The method of any one of embodiments A1 to A4, wherein the growth matrix comprises a nutrient source, wherein the nutrient source is the same or different than the substrate.

A6. The method of embodiment A5, wherein the nutrient source is different than the substrate.

A7. The method of any one of embodiments A1 to A6, wherein introducing the aqueous mist into the growth environment comprises depositing the aqueous mist onto the growth matrix, the extra-particle aerial mycelial growth, or both.

A8. The method of any one of embodiments A1 to A7, wherein the mist deposition rate is less than about 100 microliter/cm²/hour, is less than about 75 microliter/cm²/hour, is less than about 50 microliter/cm²/hour, or is less than about 25 microliter/cm²/hour.

A9. The method of embodiment A8, wherein the mist deposition rate is less than about 10 microliter/cm²/hour, is less than about 5 microliter/cm²/hour, is less than about 4 microliter/cm²/hour, is less than about 3 microliter/cm²/hour, is less than about 2 microliter/cm²/hour, or is less than about 1 microliter/cm²/hour.

A10. The method of any one of embodiments A1 to A9, wherein the CO₂ level is within a range of about 0.2% (v/v) to about 7% (v/v).

A11. The method of any one of embodiments A1 to A10, wherein the CO₂ level is greater than about 2% (v/v).

A12. The method of embodiment A11, wherein the CO₂ level is within a range of about 3% (v/v) to about 7% (v/v).

A13. The method of any one of embodiments A1 to A12, wherein the O₂ level is within a range of about 14% to about 21% (v/v).

A14. The method of any one of embodiments A1 to A13, wherein the relative humidity is at least about 95%, is at least about 96% or is at least about 97%.

A15. The method of embodiment A14, wherein the relative humidity is at least about 98%, is at least about 99%, or is about 100%.

A16. The method of any one of embodiments A1 to A15, wherein the fungus is a filamentous fungus.

A17. The method of any one of embodiments A1 to A16, wherein the incubation time period is up to about 3 weeks.

A18. The method of embodiment A17, wherein the incubation time period is within a range of about 4 days to about 17 days.

A19. The method of embodiment A17, wherein the incubation time period is within a range of about 7 days to about 16 days, is within a range of about 8 days to about 15 days, is within a range of about 9 days to about 15 days, or is within a range of about 9 days to about 14 days.

A20. The method of embodiment A17, wherein the incubation time period is about 7 days, is about 8 days, is about 9 days, is about 10 days, is about 11 days, is about 12 days, is about 13 days, is about 14 days, is about 15 days or is about 16 days.

A21. The method of any one of embodiments A1 to A20, wherein the growth environment is a dark environment.

A22. The method of any one of embodiments A1 to A21, wherein the growth environment has a temperature within a range of about 55° F. to about 100° F., or within a range of about 60° F. to about 95° F.

A23. The method of embodiment A22, wherein the growth environment temperature is within a range of about 60° F. to about 75° F., is within a range of about 65° F. to about 75° F., or is within a range of about 65° F. to about 70° F.

A24. The method of embodiment A22, wherein the growth environment temperature is within a range of about 80° F. to about 95° F., or is within a range of about 85° F. to about 90° F.

A25. The method of any one of embodiments A21 to A24, wherein the growth environment further comprises an airflow.

A26. The method of any one of embodiments A1 to A25, further comprising directing an airflow through the growth environment.

A27. The method of embodiment A25 or A26, wherein the airflow is a substantially horizontal airflow.

A28. The method of embodiment A27, wherein the substantially horizontal airflow has a velocity of no greater than about 275 linear feet per minute, has a velocity of no greater than about 175 linear feet per minute, or has a velocity of no greater than about 150 linear feet per minute.

A29. The method of embodiment A27, wherein the substantially horizontal airflow has a velocity of no greater than about 125 linear feet per minute, has a velocity of no greater than about 110 linear feet per minute, has a velocity of no greater than about 100 linear feet per minute, or has a velocity of no greater than about 90 linear feet per minute.

A30. The method of any one of embodiments A27 to A29, wherein the substantially horizontal airflow has a velocity of at least about 5, 10, 15, 20, 25, 30, 35, 40, 45 or 50 linear feet per minute.

A31. The method of any one of embodiments A6 to A30, wherein the substrate and the nutrient source each have a particle size, and wherein the substrate particle size and the nutrient particle size have a ratio within a range of about 200:1 to about 1:1, within a range of about 100:1 to about 1:1, within a range of about 50:1 to about 1:1, within a range of about 10:1 to about 1:1, or within a range of about 5:1 to about 1:1.

A32. The method of any one of embodiments A1 to A31, wherein at least a portion of the aerial mycelium has a native thickness of at least about 10 mm.

A33. The method of embodiment A32, wherein at least a portion of the aerial mycelium has a native thickness of at least about 15 mm.

A34. The method of embodiment A32 or A33, wherein the portion is at least about 10% of the aerial mycelium.

A35. The method of embodiment A32 or A33, wherein the portion is at least about 25% of the aerial mycelium.

A36. The method of embodiment A32 or A33, wherein the portion is at least about 50% of the aerial mycelium.

A37. The method of embodiment A32 or A33, wherein the portion is at least about 700% of the aerial mycelium.

A38. The method of any one of embodiments A3 to A37, wherein the aerial mycelium has a mean native density of at least about 1 pound per cubic foot (pcf) and a native moisture content of at least about 80% (w/w).

A39. The method of embodiment A38, wherein the aerial mycelium has a mean native density of at least about 2 pcf, at least about 3 pcf, at least about 4 pcf, at least about 5 pcf, at least about 6 pcf, at least about 7 pcf, at least about 8 pcf, at least about 9 pcf, or at least about 10 pcf.

A40. The method of embodiment A38, wherein the aerial mycelium has a mean native density of no greater than about 70 pcf, no greater than about 60 pcf, no greater than about 50 pcf, no greater than about 40 pcf, no greater than about 30 pcf, no greater than about 20 pcf or no greater than about 15 pcf.

A41. The method of any one of embodiments A1 to A40, wherein the aerial mycelium has an open volume of at least about 50% (v/v), at least about 60% (v/v) or at least about 70% (v/v).

A42. The method of any one of embodiments A1 to A41, wherein the ratio of the mist deposition rate to the mean mist deposition rate is within a range of about 100:1 to about 1,000:1, or within a range of about 250:1 to about 750:1.

A43. The method of embodiment A42, wherein the mist deposition rate is at least about 10 microliters/cm²/hour, or is at least about 15 microliters/cm²/hour.

A44. The method of embodiment A43, wherein the aerial mycelium has a mean native density of at least about 15 pcf.

A45. The method of embodiment A42, A43 or A44, wherein the method further comprises drying the aerial mycelium to provide a dry aerial mycelium having a moisture content of no greater than about 10% (w/w); and wherein the dry aerial mycelium has a dry density of at least about 3 pcf, or has a dry density of at least about 3.5 pcf.

A46. The method of embodiment A41, wherein the mist deposition rate is less than about 2 microliter/cm² hour, the mean mist deposition rate is less than about 1 microliter/cm²/hour, or both.

A47. The method of embodiment A46, wherein the mean mist deposition rate is within a range of about 0.2 to about 0.8 microliter/cm²/hour.

A48. The method of embodiment A46 or A47, wherein the mist deposition rate is less than about 1 microliter/cm/hour, the mean mist deposition rate is less than about 0.5 microliter/cm²/hour, or both.

A49. The method of any one of embodiments A1 to A41 and A46 to A48, wherein the mist deposition rate is at least about 0.05 microliter/cm²/hour, and the mean mist deposition rate is at least about 0.02 microliter/cm²/hour.

A50. The method of any one of embodiments A46 to A49, wherein the ratio of the mist deposition rate and the mean mist deposition rate is within a range of about 3:1 to about 1:1.

A51. The method of any one of embodiments A46 to A50, wherein the aerial mycelium has: a mean native density of at least about 1 pcf, at least about 2 pcf, at least about 3 pcf, at least about 4 pcf, or at least about 5 pcf; a mean native density of no greater than about 45 pcf; and a native moisture content of at least about 80% (w/w).

A52. The method of embodiment A51, wherein the aerial mycelium has a Kramer shear force of no greater than about 15 kilogram per gram of aerial mycelium, of no greater than about 10 kilogram/gram of aerial mycelium, or within a range of about 2 kilogram per gram to about 10 kilogram per gram of aerial mycelium.

A53. The method of any one of embodiments A46 to A52, wherein the method further comprises drying the aerial mycelium to provide a dry aerial mycelium having a moisture content of no greater than about 10% (v/v); and wherein the dry aerial mycelium has a dry density of less than about 3 pcf, less than about 2 cf or less than about 1 pcf.

A54. The method of any one of embodiments A3 to A53, further comprising terminating the incubation prior to removing the extra-particle aerial mycelial growth from the growth matrix.

A55. The method of any one of embodiments A3 to A54, further comprising terminating the incubation prior to formation of a visible fruiting body.

A56. The method of any one of embodiments A1 to A55, wherein the method further comprises terminating the incubation during a decline in aerial mycelial growth rate.

A57. The method of any one of embodiments A1 to A56, wherein the method further comprises terminating the incubation during a stationary phase of aerial mycelial growth.

A58. The method of any one of embodiments A1 to A57, wherein the method further comprises terminating the incubation prior to necrosis or death of the fungus.

A59. The method of any one of embodiments A1 to A58, wherein the method further comprises terminating the incubation after the mycelial thickness fails to substantially increase over a period of 1 day.

A60. The method of any one of embodiments A1 to A59, wherein the fungus is a species of the genus Agrocybe, Albatrellus, Amillaria, Agaricus, Bondarzewia, Cantharellus, Cerioporus, Chimacodon, Cordvceps, Fistuihna, Flammulina, Fomes, Fomitopsis, Fusarium, Grifola, Herecium, Hydnum, Hypomyces, Hypsizygus, Ischnoderma, Laetiporus, Laricifomes, Lentimda, Lentinus, Lepista, Meripilus, Morchella, Ophiocordyceps, Panellus, Piptoporus, Pleurotus, Polyporus, Pycnoporellus, Rhizopus, Schizophyllum, Stropharia, Tuber, Tvromyces or Wollfporia.

A61. The method of embodiment A60, wherein the fungus is a species of the genus Flammulina, Lentiula, Morchella or Pleurotus.

A62. The method of embodiment A61, wherein the fungus is a species of the genus Pleurotus.

A63. The method of any one of embodiments A1 to A60, wherein the fungus is an edible fungus selected from the group consisting of: Agaricus spp., Agaricus bisporus, Agaricus arvensis, Agaricus campestris, Agaricus bitorquis, Agaricus brasiliensis, Albatrellus spp., Bondarzewia berkleyii, Cantharellus spp., Cantharellus cibarius, Cerioporus squamosus, Climacodon spp., Cordyceps spp., Cordyceps militaris, Fistulina hepatica, Flammulina velutipes, Fomes spp., Fomitopsis spp., Fusarium spp., Grnfola frondosa, Herecium spp., Herecium erinaceus, Herecium americamum, Herecium abietis, Hydmim spp., Hydnmm repandum, Hydnum umbellatum, Hvpomyces lactifuorum, Hypomyces spp., Hypsizygus spp., Ischnoderma resinosum, Laetiporus spp., Laetiporus suIphureus, Laetiporus cinncinatus, Laetiporus gilbertsonii, Laetiporus conifericola, Laetiporus huroniensis, Laricifomes oficinalis, Lepista nuda, Aeripilus spp., Aferipilus gigantea, Meripilus sumstinei, Morchella spp., Morchella esculenta, Morchella angusticeps, Morchella rufobrunnea, Morchella importuna, Morchella tomentosa, Morchella elata, Morchella semilibera, Morchella Americana, Morchella punctipes, Morchella deliciosa, Morchella conica, Ophiocordyceps sinensis, Panellus spp., Panellus serotinus, Piptoporus betulina, Pleurotus spp., Pleurotus eryngii, Pleurotus ostreatus, Pleurotus tuber-regium, Pleurotus eryngii, Pleurotus pulmonarius, Pleurotus sajor-caju, Pleurotus citrinopilleatus, Pleurotus columbinus, Pleurotus djamor, Pleurotus cornmcopiae, Pleurotus dryinus, Pleurotus floridanus, Pleurotus populimus, Polyporus spp., Polyporus umbellatus, Polyporus squamosus, Pycnoporellus spp., Rhizopus oligosporus, Rhizopus oryzae, Schizophvllum commune, Stropharia rugoso-annulata, Tyromyces spp., and Wolfiporia extensa.

A64. The method of any one of embodiments A1 to A63, wherein the aerial mycelium is an edible aerial mycelium.

A65. The method of embodiment A63 or A64, wherein the fungus is Pleurotus citrinopilleatus, Pleurotus columbinus, Pleurotus cornucopiae, Pleurotus dryinus, Pleurotus djamor, Pleurotus eryngii, Pleurotus floridanus, Pleurotus ostreatus, Pleurotus populinus, Pleurotus pulmonarius, Pleurotus sajor-caju or Pleurotus tuber-regium.

A66. The method of embodiment A65, wherein the fungus is Pleurotus ostreatus.

A67. The method of embodiment A66, wherein the fungus is Pleurotus ostreatus (Jacquin: Fries) strain ATCC 58753 NRRL 2366 or Pleurotus ostreatus ATCC 56761.

A68. The method of any one of embodiments A1 to A67, wherein the aqueous mist comprises one or more solutes.

A69. The method of any one of embodiments A1 to A68, wherein the aqueous mist has a conductivity of no greater than about 1,000 microsiemens/cm, has a conductivity of no greater than about 800 microsiemens/cm, has a conductivity of no greater than about 500 microsiemens/cm, has a conductivity of no greater than about 100 microsiemens/cm, or has a conductivity of no greater than about 50 microsiemens/cm.

A70. The method of any one of embodiments A1 to A69, wherein the aqueous mist has a conductivity of no greater than about 25 microsiemens/cm, has a conductivity of no greater than about 10 microsiemens/cm, has a conductivity of no greater than about 5 microsiemens/cm, or has a conductivity of no greater than about 3 microsiemens/cm.

A71. The method of any one of embodiments A1 to A70, further comprising removing the extra-particle aerial mycelium from the growth matrix as a single contiguous object.

A72. The method of embodiment A71, thereby obtaining the aerial mycelium as a single contiguous object having a contiguous volume.

A73. The method of embodiment A71 or A72, wherein the single contiguous object is characterized as having a contiguous volume of at least about 15 cubic inches.

A74. The method of embodiment A71, A72 or A73, wherein the single contiguous object is characterized as having a series of linked hyphae over the contiguous volume.

A75. An edible aerial mycelium obtained from the method of any one of embodiments A1 to A74.

A76. An edible aerial mycelium obtained from the method of any one of embodiments A46 to A74.

A77. A system for growing an edible aerial mycelium, comprising: a growth matrix comprising a substrate and a fungal inoculum, wherein the fungal inoculum comprises a fungus; a growth environment configured to incubate the growth matrix as a solid-state culture for an incubation time period; and an atmospheric control system with an electronic controller configured to maintain a carbon dioxide (CO₂) level within the growth environment between at least about 0.02% (v/v) and less than about 8% (v/v) and to introduce aqueous mist into the growth environment throughout the incubation time period, or a portion thereof, at a mist deposition rate of less than or equal to about 150 microliter/cm²/hour, and a mean mist deposition rate over the incubation time period of less than or equal to about 3 microliter/cm²/hour.

A78. The system of embodiment A77, wherein the growth environment is maintained at a relative humidity of at least about 95%.

A79. The system of embodiment A77 or A78, wherein the growth environment comprises a misting apparatus.

A80. The system of embodiment A77, A78 or A79, wherein the system is configured to provide a substantially horizontal airflow across the growth matrix.

A81. An edible product comprising an edible aerial mycelium, wherein: the aerial mycelium is an edible aerial mycelium having: a mean native density within a range of about 1 to about 70 pounds per cubic foot (pcf); a native moisture content of at least about 80% (w/w); and a Kramer shear force of no greater than about 15 kilogram per gram of edible aerial mycelium; wherein at least a portion of the aerial mycelium has a native thickness of at least about 10 mm.

A82. The edible product of embodiment A81, wherein the aerial mycelium does not contain a fruiting body.

A83. The edible product of embodiment A81 or A82, wherein at least a portion of the aerial mycelium has a native thickness of at least about 15 mm.

A84. The edible product of embodiment A81 or A82, wherein at least a portion of the aerial mycelium has a native thickness of at least about 20 mm.

A85. The edible product of any one of embodiments A81 to A84, wherein the portion is at least about 10% of the aerial mycelium.

A86. The edible product of embodiment A85, wherein the portion is at least about 25% of the aerial mycelium.

A87. The edible product of embodiment A85, wherein the portion is at least about 50%, is at least about 60%, or is at least about 70% of the aerial mycelium.

A88. The edible product of embodiment A85, wherein the portion is at least about 800% of the aerial mycelium.

A89. The edible product of any one of embodiments A81 to A88, wherein the aerial mycelium has a mean hyphal width of at most about 20 microns, at most about 15 microns, or within a range of about 0.2 to about 15 microns.

A90. The edible product of any one of embodiments A81 to A89, wherein the aerial mycelium has a mean native density of at least about 1 pound per cubic foot (pcf), at least about 2 pcf, at least about 3 pcf, at least about 4 pcf or about 5 pcf.

A91. The edible product of any one of embodiments A81 to A90, wherein the aerial mycelium has a mean native density of at least about 10 pcf.

A92. The edible product of any one of embodiments A81 to A91, wherein the aerial mycelium has a mean native density of no greater than about 60 pcf, no greater than about 50 pcf, no greater than about 40 pcf, no greater than about 30 pcf, no greater than about 20 pcf or no greater than about 15 pcf.

A93. The edible product of any one of embodiments A81 to A92, wherein the aerial mycelium has an open volume of at least about 50% (v/v), at least about 60% (v/v) or at least about 70% (v/v).

A94. The edible product of any one of embodiments A81 to A93, wherein the aerial mycelium is a growth product of an edible fungus.

A95. The edible product of embodiment A94, wherein the edible fungus is a species of the genus Agrocybe, Albatrellus, Amillaria, Agaricus, Bondarzewia, Cantharellus, Cerioporus, Clinacodon, Cordyceps, Fistulina, Flammulina, Fones, Fomitopsis, Fusarium, Grifola, Herecium, Hydnum, Hypomyces, Hypsizygus, Ischnoderma, Laetiporns, Laricifomes, Lentimda, Lentinus, Lepista, Meripilus, Morchella, Ophiocordyceps, Panelhus, Piptoporus, Pleurotus, Polyporus, Pycnoporellus, Rhizopus, Schizophyllum, Stropharia, Trametes, Tuber, Tyromyces or Wolfiporia.

A96. The edible product of embodiment A95, wherein the edible fungus is Pleurotus citrinopilleatus, Pleurotus columbinus, Pleurotus corntucopiae, Pleurotus dryinus, Pleurotus djamor, Pleurotus eryngii, Pleurotus floridanus, Pleurotus ostreatus, Pleurotus populinus, Pleurotus pulmonarius, Pleurotus sajor-caju or Pleurotus tuber-regium.

A97. The edible product of embodiment A96, wherein the edible fungus is Pleurolus ostreatus.

A98. The edible product of embodiment A97, wherein the fungus is Pleurotus ostreatus (Jacquin: Fries) strain ATCC 58753 NRRL 2366 or Pleurotus ostreatus ATCC 56761.

A99. The edible product of any one of embodiments A81 to A98, wherein the edible product consists of the edible aerial mycelium.

A100. The edible product of any one of embodiments A81 to A99, wherein the edible product further comprises one or more additives.

A101. The edible product of embodiment A100, wherein the additive is a fat, a protein, an amino acid, a flavorant, an aromatic agent, a mineral, a vitamin, a micronutrient, a colorant or a preservative; or a combination thereof.

A102. The edible product of embodiment A101, wherein the fat is almond oil, animal fat, avocado oil, butter, canola oil, coconut oil, corn oil, grapeseed oil, hempseed oil, lard, mustard oil, olive oil, palm oil, peanut oil, rice bran oil, safflower oil, soybean oil, sunflower seed oil, vegetable oil, vegetable shortening or animal fat; or a combination thereof; and wherein the animal fat is optionally pork fat, chicken fat or duck fat; optionally, each said oil is a refined oil.

A103. The edible product of embodiment A102, wherein the protein is a heme protein.

A104. The edible product of embodiment A101, wherein the amino acid is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine; or a combination thereof.

A105. The edible product of embodiment A101, wherein the flavorant is a smoke flavorant, umami, maple, a salt, a sweetener, a spice, or a combination thereof.

A106. The edible product of embodiment A105, wherein the umami is a glutamate; optionally, the glutamate is sodium glutamate.

A107. The edible product of embodiment A105, wherein the salt is sea salt.

A108. The edible product of embodiment A105, wherein the spice is jalepeno, capsaicin or paprika, or a combination thereof.

A109. The edible product of embodiment A105, wherein the smoke flavorant is a liquid smoke flavorant, a natural hickory smoke or an artificial hickory smoke, or a combination thereof.

A110. The edible product of embodiment A101, wherein the aromatic agent is allicin.

A111. The edible product of embodiment A101, wherein the mineral is iron, magnesium, manganese, selenium, zinc, calcium, sodium, potassium, molybdenum, iodine or phosphorus, or a combination thereof.

A112. The edible product of embodiment A101, wherein the vitamin is a ascorbic acid (vitamin C), biotin, a retinoid, a carotene, vitamin A, thiamine (vitamin B1), riboflavin (vitamin B2), pantothenic acid (vitamin B5), pyridoxine (vitamin B6), folate, folic acid (vitamin B9), cobalamine (vitamin B12), choline, calciferol (vitamin D), alpha-tocopherol (vitamin E) or phylloquinone (menadione, vitamin K), or a combination thereof.

A113. The edible product of embodiment A101, wherein the colorant is beet extract or paprika, or a combination thereof.

A114. The edible product of any one of embodiments A81 to A113, wherein the product contains substantially no amount of an artificial preservative.

A115. The edible product of any one of embodiments A81 to A114, wherein the product contains substantially no amount of an artificial colorant.

A116. The edible product of any one of embodiments A81 to A115, wherein the aerial mycelium has a protein content within a range of about 21% to about 41% (w/w), a fat content of less than about 7% (w/w), a carbohydrate content within a range of about 37% to about 70% (w/w), and a total dietary fiber content within a range of about 15% to about 28% (w/w); wherein each said percentage is based on a dry mass of the aerial mycelium.

A117. The edible product of embodiments A116, wherein the protein content is within a range of about 25% to about 33% (w/w), the fat content is within a range of about 2.5% and about 6.5% (w/w), the carbohydrate content is within a range of about 43% to about 65% (w/w), and the total dietary fiber content within a range of about 17% to about 26% (w/w).

A118. The edible product of any one of embodiments A81 to A117, wherein the edible product is a food product.

A119. The edible product of embodiment A118, wherein the food product is a mycelium-based food product.

A120. The edible product of embodiment A118 or A119, wherein the food product is a whole muscle meat alternative.

A121. The edible product of embodiment A118, A119 or A120, wherein the food product is a mycelium-based bacon product.

A122. The edible product of embodiment A118, wherein the food product is a food ingredient.

A123. The edible product of embodiment A122, wherein the food ingredient is suitable for use in manufacturing a mycelium-based food product, or wherein the food ingredient is for use in manufacturing a mycelium-based food product.

A124. The edible product of embodiment A123, wherein the mycelium-based food product is a whole muscle meat alternative.

A125. The edible product of embodiment A123, wherein the mycelium-based food product is a mycelium-based bacon product.

A126. The edible product of any one of embodiments A1 to A125, wherein the aerial mycelium is a single contiguous object having a contiguous volume.

A127. The edible product of embodiment A126, wherein the contiguous object is characterized as having a contiguous volume of at least about 15 cubic inches.

A128. The edible product of embodiment A126 or A127, wherein the contiguous object is characterized as having series of linked hyphae over the contiguous volume.

A129. The edible product of embodiment A118, wherein the food product is a structured alternative for carbohydrate or animal protein structures; optionally, the food product is mycelium-based eggs, mycelium-based pasta or mycelium-based confections.

A130. The edible product of any one of embodiments A81 to A129, provided that the aerial mycelium is not a growth product of a fungal species of the genus Ganoderma.

A131. A method of making an edible appressed mycelium, comprising: providing a growth matrix comprising a substrate and a fungal inoculum, wherein the fungal inoculum comprises a fungus; incubating the growth matrix as a solid-state culture in a growth environment for an incubation time period; provided that the growth environment excludes mist; thereby producing extra-particle appressed mycelial growth from the growth matrix.

A132. The method of embodiment A131, wherein: the growth environment comprises a growth atmosphere having a relative humidity, an oxygen (O₂) level and a carbon dioxide (CO₂) level, wherein the CO₂ level is at least about 0.02% (v/v) and less than about 8% (v/v).

A133. The method of embodiment A131 or A132, further comprising removing the extra-particle appressed mycelial growth from the growth matrix, thereby providing an appressed mycelium.

A134. The method of embodiment A131, A132 or A133, wherein:

(a) the carbon dioxide level is within a range of about 0.2% and 7% (v/v), and the appressed mycelium does not contain a visible fruiting body; or
(b) the carbon dioxide level is within a range of about 0.02% and 7%, and

(i) the incubation time period ends no later than when a visible fruiting body forms;

(ii) the incubation time period ends when a visible fruiting body forms; or

(iii) the aerial mycelium does not contain a visible fruiting body.

A135. The method of any one of embodiments A131 to A134, wherein the growth matrix comprises a nutrient source, wherein the nutrient source is the same or different than the substrate.

A136. The method of embodiment A135, wherein the nutrient source is different than the substrate.

A137. The method of any one of embodiments A131 to A136, wherein the CO₂ level is within a range of about 0.2 to about 7% (v/v).

A138. The method of embodiment A137, wherein the CO₂ level is greater than about 2% (v/v).

A139. The method of any one of embodiments A131 to A138, wherein the O₂ level is within a range of about 14% to about 21% (v/v).

A140. The method of any one of embodiments A131 to A139, wherein the relative humidity is at least about 95%, is at least about 96% or is at least about 97%.

A141. The method of embodiment A140, wherein the relative humidity is at least about 98%, is at least about 99%, or is about 100%.

A142. The method of any one of embodiments A131 to A141, wherein the fungus is a filamentous fungus.

A143. The method of any one of embodiments A131 to A142, wherein the incubation time period is up to about 3 weeks.

A144. The method of embodiment A143, wherein the incubation time period is within a range of about 4 days to about 17 days.

A145. The method of embodiment A143, wherein the incubation time period is within a range of about 7 days to about 16 days, is within a range of about 8 days to about 15 days, is within a range of about 9 days to about 15 days, or is within a range of about 9 days and about 14 days.

A146. The method of embodiment A143, wherein the incubation time period is about 7 days, is about 8 days, is about 9 days, is about 10 days, is about 11 days, is about 12 days, is about 13 days, is about 14 days, is about 15 days or is about 16 days.

A147. The method of any one of embodiments A131 to A146, wherein the growth environment is a dark environment.

A148. The method of any one of embodiments A131 to A147, wherein the growth environment has a temperature within a range of about 55° F. to about 100° F., or within a range of about 60° F. to about 95° F.

A149. The method of embodiment A148, wherein the growth environment temperature is within a range of about 60° F. to about 75° F., is within a range of about 65° F. to about 75° F., or is within a range of about 65° F. to about 70° F.

A150. The method of embodiment A148, wherein the growth environment temperature is within a range of about 80° F. to about 95° F., or is within a range of about 85° F. to about 90° F.

A151. The method of any one embodiments A131 to A150, wherein the growth environment further comprises an airflow.

A152. The method of any one of embodiments A131 to A151, further comprising directing an airflow through the growth environment.

A153. The method of embodiment A131 or A152, wherein the airflow is a substantially horizontal airflow.

A154. The method of embodiment A153, wherein the substantially horizontal airflow has a velocity of no greater than about 275 linear feet per minute, has a velocity of no greater than about 175 linear feet per minute, or has a velocity of no greater than about 150 linear feet per minute.

A155. The method of embodiment A153, wherein the substantially horizontal airflow has a velocity of no greater than about 125 linear feet per minute, has a velocity of no greater than about 110 linear feet per minute, has a velocity of no greater than about 100 linear feet per minute, or has a velocity of no greater than about 90 linear feet per minute.

A156. The method of any one of embodiments A153 to A155, wherein the substantially horizontal airflow has a velocity of at least about 5, 10, 15, 20, 25, 30, 35, 40, 45 or 50 linear feet per minute.

A157 The method of any one of embodiments A136 to A156, wherein the substrate and the nutrient source each have a particle size, and wherein the substrate particle size and the nutrient particle size have a ratio within a range of about 200:1 to about 1:1, within a range of about 100:1 to about 1:1, within a range of about 50:1 to about 1:1, within a range of about 10:1 to about 1:1, or within a range of about 5:1 to about 1:1.

A158. The method of any one of embodiments A133 to A157, wherein the appressed mycelium has a mean native thickness of no greater than about 3 mm, and a native moisture content of less than about 80% (w/w), or a native moisture content of no greater than about 78% (w/w).

A159. The method of any one of embodiments A131 to A158, wherein the method further comprises drying the appressed mycelium to provide a dry appressed mycelium having a moisture content of no greater than about 10% (v/v), wherein the dry appressed mycelium has a dry density within a range of about 2.8 to about 8 pounds per cubic foot (pcf).

A160. The method of embodiment A159, wherein the dry appressed mycelium has a mean dry density within a range of about 3.5 to about 8 pcf.

A161. The method of embodiment A159, wherein the dry appressed mycelium has a mean dry density within a range of about 5 to about 6 pcf.

A162. The method of any one of embodiments A131 to A161, further comprising terminating the incubation prior to removing the extra-particle appressed mycelial growth from the growth matrix.

A163. The method of any one of embodiments A131 to A162, further comprising terminating the incubation prior to formation of a visible fruiting body.

A164. The method of any one of embodiments A131 to A163, wherein the method further comprises terminating the incubation during a decline in appressed mycelial growth rate.

A165. The method of any one of embodiments A131 to 163, wherein the method further comprises terminating the incubation during a stationary phase of appressed mycelial growth.

A166. The method of any one of embodiments A131 to A165, wherein the method further comprises terminating the incubation prior to necrosis or death of the fungus.

A167. The method of any one of embodiments A131 to A166, wherein the fungus is a species of the genus Agrocybe, Albatrellus, Amillaria, Agaricus, Bondarzewia, Cantharellus, Cerioporus, Climacodon, Cordyceps, Fistulina, Flammulina, Fomes, Fomitopsis, Fusarium, Grifola, Herecium, Hydnum, Hypomyces, Hypsizygus, Ischnoderma. Laetiporus, Laricifomes, Lentinula, Lentinus, Lepista, Meripilus, Morchella, Ophiocordyceps, Panellus, Piptoporus, Pleurotus, Polyporus, Pycnoporellus, Rhizopus, Schizophyllum, Stropharia. Trametes, Tuber, Tyromyces or Wolfiporia.

A168. The method of any one of embodiments A131 to A167, wherein the fungus is a species of the genus Flammulina, Lentinula, Morchella or Pleurotus.

A169. The method of any one of embodiments A131 to A167, wherein the fungus is an edible fungus selected from the group consisting of: Agaricus spp., Agaricus bisporus, Agaricus arvensis, Agaricus campestris, Agaricus bilorquis, Agaricus brasiliensis, Albatrellus spp., Bondarzewia berkleyii, Canitharellus spp., Cantharellus cibarius, Cerioporus squamosus, Climacodon spp., Cordyceps spp., Cordyceps militaris, Fistulina hepatica, Flammulina velutipes, Fomes spp., Fomitopsis spp., Fusarium spp., Grifola frondosa, Herecium spp., Herecium erinaceus, Herecium americamum, Herecium abietis, Hyndnum spp., Hydnum repandum, Hydnum umbellatum, Hypomyces lactifuorum, Hypomyces spp., Hypsizygus spp., Ischnoderma resinosum, Laetiporus spp., Laetiporus sulphureus, Laetiporus cinncinatus, Laetiporus gilbertsonii, L. aetiporus conifericola, Laetiporus huroniensis, Laricifomes oficinalis, Lepista nuda, Meripilus spp., Meripilus gigantea, Meripilus sumstinei, Morchella spp., Morchella esculenta, Morchella angusticeps, Morchella rufobrunnea, Morchella importuna, Morchella tomentosa, Morchella elata, Morchella semilibera, Morchella Americana, Morchella punctipes, Morchella deliciosa, Morchella conica, Ophiocordyceps sinensis, Panellus spp., Panellus serotinus, Piptoporus betulina, Pleurotus spp., Pleurotus eryngii, Pleurotus ostreatus, Pleurotus tuber-regium, Pleurotus eryngii, Pleurotus pulmonarius, Pleurotus sajor-caju, Pleurotus citrinopilleatus, Pleurotus columbinus, Pleurotus djamor, Pleurotus cornucopiae, Pleurotus dryinus, Pleurotus floridanus, Pleurotus populinus, Polyporus spp., Polyporus umbellatus, Polyporus squamosus, Pycnoporellus spp., Rhizopus oligosporus, Rhizopus oryzae, Schizophyllum commune, Stropharia rugoso-anuluata, Tyromyces spp., Trametes spp. and Wolfiporia extensa.

A170. The method of any one of embodiments A131 to A169, wherein the appressed mycelium is an edible appressed mycelium.

A171. The method of embodiment A169 or A170, wherein the fungus is Pleurotus citrinopilleatus, Pleurotus columbinus, Pleurotus cornucopiae, Pleurotus dryinus, Pleurotus djamor, Pleurotus eryngii, Pleurotus floridamus, Pleurotus ostreatus, Pleurotus popiminus, Pleurotus pulmonarius, Pleurotus sajor-caju or Pleurotus tuber-regium.

A172. The method of embodiment A171, wherein the fungus is Pleurotus ostreatus.

A173. The method of embodiment A172, wherein the fungus is Pleurotus ostreatus (Jacquin: Fries) strain ATCC 58753 NRRL 2366 or Pleurotus ostreatus ATCC 56761.

A174. The method of any one of embodiments A133 to A173, further comprising removing the extra-particle appressed mycelium from the growth matrix as a single contiguous sheet.

A175. The method of embodiment A174, thereby obtaining the appressed mycelium as the single contiguous sheet having a contiguous surface area.

A176. The method of embodiment A174 or A175, wherein the single contiguous sheet is characterized as having a contiguous surface area of at least about 16 square inches.

A177. The method of embodiment A174, A175 or A176, wherein the single contiguous sheet is characterized as a series of linked hyphae over the contiguous surface area.

A178. The method of any one of embodiments A131 to A177, wherein the appressed mycelium is suitable for use in the manufacture of a food product.

A179. The method of any one of embodiments A131 to A178, wherein the appressed mycelium is for use in the manufacture of a food product.

A180. An edible appressed mycelium obtained from a method of any one of embodiments A131 to A179.

A181. The method of any one of embodiments A1 to A74 and A131 to A179, wherein the growth matrix further comprises at least one additive.

A182. The method of embodiment A181, wherein the additive is a component of the nutrient source.

A183. The method of embodiment A181 or A182, wherein the additive is the nutrient source.

A184. The method of embodiment A181 or A182, wherein the additive is a micronutrient, a mineral, an amino acid, a peptide, a protein, allicin or a combination thereof.

A185. The method of any one of embodiments A1 to A74 and A131 to A179, further comprising adding at least one additive to the mycelium or to the extra-particle mycelial growth.

A186. The method of embodiment A185, wherein adding the additive occurs during the incubation time period.

A187. The method of embodiment A185, wherein adding the additive occurs after the incubation time period.

A188. The method of embodiment A187, wherein adding the additive occurs after removing the extra-particle mycelium from the growth matrix.

A189. The method of any one of embodiments A185 to A188, wherein the additive is a fat, a protein, an amino acid, a flavorant, an aromatic agent, a mineral, a vitamin, a micronutrient, a colorant or a preservative; or a combination thereof.

A190. The method of embodiment A188, wherein the additive is the additive of any one of embodiments A102 to A113, or any additive as disclosed herein.

A191. The method of any one of embodiments A1 to A190, wherein the method excludes grinding the mycelium.

A192. The method of any one of embodiments A1 to A190, wherein the method excludes mincing the mycelium.

A193. The method of any one of embodiments A1 to A192, wherein the method excludes extruding the mycelium.

A200. An edible mycelium obtained from a method of any one of embodiments A181 to A193.

A201. A method of preparing edible mycelium-based bacon, the method comprising: providing an edible aerial mycelium having: a mean density within a range of about 1 to about 45 pcf, about 2 pcf to about 45 pcf, about 3 pcf to about 45 pcf, about 4 pcf to about 45 pcf or about 5 pcf to about 45 pcf; a moisture content of at least about 80% (w/w); and a Kramer shear force of no greater than about 15 kilogram per gram of the edible aerial mycelium; wherein at least a portion of the edible aerial mycelium has a thickness of at least about 15 mm; and cutting the edible aerial mycelium into a plurality of strips.

A202. The method of embodiment A201, wherein the mean density is a mean native density, the moisture content is a native moisture content, and the thickness is a native thickness.

A203. The method of embodiment A201 or A202, wherein the portion is at least about 10% of the aerial mycelium, or is at least about 25% of the aerial mycelium.

A204. The method of embodiment A201 or A202, wherein the portion is at least about 50% of the aerial mycelium, or is at least about 70% of the aerial mycelium.

A205. The method of any one of embodiments A201 to A204, wherein cutting the edible aerial mycelium into the plurality of strips comprises cutting the edible aerial mycelium in a direction substantially parallel to the direction of aerial mycelial growth.

A206. The method of any one of embodiments A201 to A205, wherein the method further comprises compressing the plurality of strips.

A207. The method of embodiment A206, wherein compressing the plurality of strips comprises applying pressure to at least one strip, thereby providing at least one compressed strip.

A208. The method of embodiment A207, wherein the method further comprises perforating the at least one compressed strip.

A209. The method of any one of embodiments A201 to A208, wherein the aerial mycelium further comprises an additive.

A210. The method of embodiment A209, wherein the additive is a fat, a protein, an amino acid, a flavorant, an aromatic agent, a mineral, a vitamin, a micronutrient, a colorant or a preservative; or a combination thereof; or the additive is as described in any one of embodiments A102 to A113.

A211. A method of making an edible aerial mycelium, comprising: providing a growth matrix comprising a substrate, a nutrient source and a fungal inoculum, wherein the fungal inoculum comprises a filamentous fungus; incubating the growth matrix as a solid-state culture in a growth environment for an incubation time period of up to about 3 weeks, wherein the growth environment comprises a growth atmosphere having a carbon dioxide (CO₂) level within a range of about 0.2% (v/v) to about 7% (v/v), and a relative humidity of at least about 95%; introducing aqueous mist into the growth environment throughout the incubation time period, or a portion thereof, wherein the aqueous mist has a mist deposition rate of no greater than about 2 microliter/cm²/hour, and a mean mist deposition rate of no greater than about 1 microliter/cm²/hour, thereby producing extra-particle aerial mycelial growth from the growth matrix; and removing the extra-particle aerial mycelial growth from the growth matrix, thereby providing an edible aerial mycelium; wherein the filamentous fungus is an edible species of fungus.

A212. The method of embodiment A211, wherein introducing the aqueous mist into the growth environment comprises depositing the aqueous mist onto an exposed surface of the growth matrix, an exposed surface of the aerial mycelial growth, or both.

A213. The method of embodiment A211 or A212, wherein the carbon dioxide level is within a range of about 3% (v/v) to about 7% (v/v).

A214. The method of any one of embodiments A211 to A213, wherein the O₂ level is within a range of about 14% to about 21% (v/v).

A215. The method of any one of embodiments A211 to A214, wherein the relative humidity is at least about 98%, is at least about 99%, or is about 100%.

A216. The method of any one of embodiments A211 to A215, further comprising removing the extra-particle aerial mycelium from the growth matrix as a single contiguous object, thereby obtaining the edible aerial mycelium as a single contiguous object having a contiguous volume, wherein the contiguous volume is at least about 15 cubic inches.

A217. The method of embodiment A216, wherein the single contiguous object is characterized as having a series of linked hyphae over the contiguous volume.

A218. The method of any one of embodiments A211 to A217, further comprising directing a substantially horizontal airflow through the growth environment.

A219. The method of embodiment A218, wherein the substantially horizontal airflow has a velocity of no greater than about 175 linear feet per minute, no greater than about 125 linear feet per minute, has a velocity of no greater than about 110 linear feet per minute, has a velocity of no greater than about 100 linear feet per minute, or has a velocity of no greater than about 90 linear feet per minute.

A220. The method of any one of embodiments A218 to A219, wherein the substantially horizontal airflow has a velocity of at least about 5, 10, 15, 20, 25, 30, 35, 40, 45 or 50 linear feet per minute.

A221. The method of any one of embodiments A211 to A220, wherein the substrate and the nutrient source each have a particle size, and wherein the substrate particle size and the nutrient particle size have a ratio within a range of about 200:1 to about 1:1, within a range of about 100:1 to about 1:1, within a range of about 50:1 to about 1:1, within a range of about 10:1 to about 1:1, or within a range of about 5:1 to about 1:1.

A222. The method of any one of embodiments A211 to A221, wherein the mean mist deposition rate is within a range of about 0.2 to about 0.8 microliter/cm²/hour.

A223. The method of any one of embodiments A211 to A222, wherein the mist deposition rate is less than about 1 microliter/cm² hour, the mean mist deposition rate is less than about 0.5 microliter/cm²/hour, or both.

A224. The method of any one of embodiments A211 to A223, wherein the mist deposition rate is at least about 0.05 microliter/cm²/hour, and the mean mist deposition rate is at least about 0.02 microliter/cm² hour.

A225. The method of any one of embodiments A211 to A224, wherein the mist deposition rate and the mean mist deposition rate have a ratio within a range of about 3:1 to about 1:1.

A226. The method of any one of embodiments A211 to A225, wherein the edible fungal species is a species of the genus Flammulina, Lentinula, Morchella or Pleurotus.

A227. The method of any one of embodiments A211 to A226, wherein the fungus is a species of the genus Pleurotus.

A228. The method of embodiment A227, wherein the fungus is Pleurotus citrinopilleatus. Pleurotus columbinus, Pleurotus cornucopiae, Pleurotus dryinus, Pleurotus djamor, Pleurotus eryngii, Pleurotus floridanus, Pleurotus ostreatus, Pleurotus populinus. Pleurotus pulmonarius, Pleurotus sajor-caju or Pleurotus tuber-regium.

A229. The method of embodiment A228, wherein the fungus is Pleurotus ostreatus.

A230. The method of any one of embodiments A211 to A229, wherein the aqueous mist comprises at least one solute.

A231. The method of any one of embodiments A211 to A230, wherein the aqueous mist has a conductivity of no greater than about 1,000 microsiemens/cm, has a conductivity of no greater than about 800 microsiemens/cm, has a conductivity of no greater than about 500 microsiemens/cm, has a conductivity of no greater than about 100 microsiemens/cm, or has a conductivity of no greater than about 50 microsiemens/cm.

A232. The method of any one of embodiments A211 to A231, wherein the aqueous mist has a conductivity of no greater than about 25 microsiemens/cm, has a conductivity of no greater than about 10 microsiemens/cm, has a conductivity of no greater than about 5 microsiemens/cm, or has a conductivity of no greater than about 3 microsiemens/cm.

A233. The method of any one of embodiments A211 to A232, wherein the incubation time period is within a range of about 4 days to about 17 days, or is within a range of about 4 to about 14 days.

A234. The method of any one of embodiments A211 to A232, wherein the incubation time period is within a range of about 7 days to about 17 days, is within a range of about 7 days to about 16 days, is within a range of about 8 days to about 15 days, is within a range of about 9 days to about 15 days, or is within a range of about 9 days and about 14 days.

A235. The method of any one of embodiments A211 to A232, wherein the incubation time period is about 7 days, is about 8 days, is about 9 days, is about 10 days, is about 11 days, is about 12 days, is about 13 days, is about 14 days, is about 15 days or is about 16 days.

A236. The method of any one of embodiments A211 to A232, wherein the incubation time period is within a range of about 8 to about 14 days.

A237. The method of embodiment A236, wherein the incubation time period is 13 or 14 days.

A238. The method of embodiment A237, wherein the incubation time period is 9 days.

A239. The method of any one of embodiments A211 to A238, wherein the carbon dioxide level is within a range of about 4% (v/v) to about 6% (v/v).

A240. The method of any one of embodiments A211 to A239, wherein the carbon dioxide level is about 5% (v/v).

A241. The method of any one of embodiments A211 to A240, wherein the growth environment is a dark environment.

A242. The method of any one of embodiments A211 to A241, wherein the growth environment has a temperature within a range of about 60° F. to about 95° F., is within a range of about 60° F. to about 75° F., is within a range of about 65° F. to about 75° F., or is within a range of about 65° F. to about 70° F.

A243. The method of any one of embodiments A211 to A241, wherein the growth environment has a temperature within a range of about 80° F. to about 95° F., or is within a range of about 85° F. to about 90° F.

A244. An edible aerial mycelium obtained from a method of any one of embodiments A211 to A243.

A245. An edible mycelium-based product comprising an edible aerial mycelium, wherein the edible aerial mycelium has: a mean native density within a range of about 1 to about 50 pounds per cubic foot (pcf), about 2 pcf to about 50 pcf, about 3 pcf to about 50 pcf, about 4 pcf to about 50 pcf or about 5 pcf to about 50 pcf; a native moisture content of at least about 80% (w/w); a Kramer shear force of no greater than about 15 kilogram per gram of aerial mycelium; and a native thickness of at least about 20 mm over at least 90% of the aerial mycelium; wherein the aerial mycelium does not contain a fruiting body.

A246. The edible mycelium-based product of embodiment A245, wherein the aerial mycelium is a growth product of a fungal species of the genus Pleurotus.

A247. The edible mycelium-based product of embodiment A246, wherein the fungal species is Pleurotus ostreatus.

A248. The edible mycelium-based product of embodiment A247, wherein the fungal species is Pleurotus ostreatus (Jacquin: Fries) strain ATCC 58753 NRRL 2366 or Pleurotus ostreatus ATCC 56761.

A249. The edible mycelium-based product of any one of embodiments A245 to A248 having a protein content within a range of about 25% to about 33% (w/w), a fat content within a range of about 2.5% and about 6.5% (w/w), a carbohydrate content within a range of about 43% to about 65% (w/w), and a total dietary fiber content within a range of about 17% to about 26% (w/w).

A250. The edible mycelium-based product of any one of embodiments A249 to A249, wherein the product consists of the edible aerial mycelium.

A251. The edible mycelium-based product of any one of embodiments A245 to A250, wherein the edible aerial mycelium is a food ingredient suitable for the manufacture of an edible mycelium-based meat alternative product.

A252. The edible mycelium-based product of any one of embodiments A245 to A250, wherein the edible aerial mycelium is a food ingredient for the manufacture of an edible mycelium-based meat alternative product.

A253. The edible mycelium-based product of any one of embodiments A245 to A250, wherein the edible aerial mycelium is a food ingredient suitable for the manufacture of an edible mycelium-based bacon product.

A254. The edible mycelium-based product of any one of embodiments A245 to A250, wherein the edible aerial mycelium is a food ingredient for the manufacture of an edible mycelium-based bacon product.

A255. The edible aerial mycelium-based product of any one of embodiments A245 to A254, wherein the aerial mycelium has a Kramer shear force of no greater than about 10 kilogram per gram of aerial mycelium.

A256. The edible aerial mycelium-based product of any one of embodiments A245 to A255, wherein the aerial mycelium has a Kramer shear force within a range of about 2 kilogram per gram to about 10 kilogram per gram of aerial mycelium.

Some further nonlimiting embodiments of the disclosure follow: B1. The method of any one of embodiments A1 to A74, A131 to A179 and A181 to A243, wherein introducing the mist into the growth environment comprises releasing the mist from a misting apparatus.

B2. The method of embodiment B1, wherein the growth environment comprises a misting apparatus.

B3. The method of embodiment B1 or B2, wherein the misting apparatus is a high pressure misting pump, a nebulizer, an aerosol generator or aerosolizer, a mist generator, an ultrasonic nebulizer, an ultrasonic aerosol generator or aerosolizer, an ultrasonic mist generator, a dry fog humidifier, an ultrasonic humidifier or an atomizer misting system (including but not limited to a “misting puck”), essentially as described in WO 2019/099474 A1, the entire content of which is hereby incorporated by reference in its entirety, or a print head configured to deposit mist, such as a 3D printer, essentially as described in U.S. patent application Ser. No. 16/688,699, the entire content of which is hereby incorporated by reference in its entirety.

B4. The method of embodiment B1 or B2, wherein the mist is introduced into the growth environment via modulation of growth environment atmospheric pressure, temperature and/or relative humidity, or via modulation of the growth atmosphere dew point.

B5. The method of embodiment B1 or B2, wherein the misting apparatus is the same or different than an apparatus that controls relative humidity of the growth environment.

B6. The method of any one of embodiments B1 to B5, wherein the total volume of aqueous mist introduced into the growth environment throughout the incubation period is less about 200 microliters/cm².

B7. The method of any one of embodiments B1 to B6, wherein the total volume of aqueous mist introduced into the growth environment throughout the incubation period is less than or equal to about 100 microliters/cm².

B8. The method of any one of embodiments B1 to B6, wherein the total volume of aqueous mist introduced into the growth environment throughout the incubation period is at least about 5 microliters/cm².

B9. The method of any one of embodiments B1 to B8, wherein the growth atmosphere has an atmospheric pressure within a range of about 27 to about 31 inches of mercury (Hg), within a range of about 29 to about 31 inches Hg, or of about 29.9 inches Hg.

B10. The method of any one of embodiments B1 to B9, wherein the method comprises terminating the incubation.

B11. The method of embodiment B10, wherein terminating the incubation comprises exposing the aerial mycelium to a terminal environment, wherein the terminal environment is different from the growth environment.

B12. The method of embodiment B11, wherein said terminal environment has one or more conditions that differ from corresponding conditions of the growth environment.

B13. The method of embodiment B12, wherein the one or more terminal environmental conditions is selected from the group consisting of relative humidity, misting condition, temperature, carbon dioxide level and oxygen level; and combinations thereof; wherein the terminal environmental misting condition is an absence of mist or a reduction in a mist deposition rate.

B14. The method of any one of embodiments B11 to B13, wherein exposing the growth matrix to the terminal environment comprises physically moving the aerial mycelium from the growth environment to the terminal environment.

B15. The method of any one of embodiments B11 to B13, wherein exposing aerial mycelium to the terminal environment comprises modifying one or more conditions of the growth environment, thereby providing the terminal environment.

B16. The method of embodiment B10, wherein terminating the incubation comprises restoring the growth environment to ambient environmental conditions.

B17. The method of anyone of embodiments B1 to B16, further comprising placing the growth matrix inside a tool.

B18. The method of embodiment B17, wherein the tool has a base having a surface area and a wall having a height.

B19. The method of embodiment B18, wherein the base has a surface area of at least about 1 square inch.

B20. The method of embodiment B18 or B19, wherein the tool has a volume of at least about 1 cubic inch.

B21. The method of embodiment B20, wherein the tool has a volume of at least about 100 cubic inches, at least about 200 cubic inches, at least about 300 cubic inches, at least about 400 cubic inches or at least about 500 cubic inches.

B22. The method of any one of embodiments B17 to B21, wherein the tool has a base having a surface area of at most about 2000 square feet.

B23. The method of any one of embodiments B1 to B16, further comprising placing the growth matrix on a planar surface.

B24. The method of embodiment B23, wherein the planar surface is a tray, a sheet, a table or a conveyer belt.

B25. The method of embodiment B24, wherein the planar surface has a surface area, and wherein the surface area is at most about 2000 square feet.

B26. The method of any one of embodiments B1 to B25, wherein the growth environment is an enclosed growth chamber.

B27. The method of any one of embodiments B1 to B26, wherein the substrate contains moisture.

B28. The method of embodiment B27, wherein the substrate has a moisture content within a range of about 45% to about 75% (w/w).

B29. The method of embodiment B28, wherein the moisture content is within a range of about 60% to about 65% (w/w).

B30. The method of any one of embodiments B1 to B29, wherein the method further comprises sterilizing or pasteurizing the substrate (a) prior to providing the growth matrix, or (b) prior to inoculating the substrate or a growth media with the fungal inoculum, wherein said growth media comprises said substrate.

B31. The method of embodiment B30, wherein the sterilization or pasteurization comprises heat sterilization, steam sterilization, or irradiation with electromagnetic radiation; optionally, the electromagnetic radiation comprises gamma rays, X-rays, UV or UV-visible radiation.

B32. The method of any one of embodiments B1 to B31, wherein the substrate is a solid or a gel.

B33. The method of embodiment B32, wherein the substrate is a natural substrate. B34. The method of embodiment B33, wherein the natural substrate comprises a lignocellulosic material; optionally, the natural substrate consists essentially of a lignocellulosic substrate, or consists of a lignocellulosic substrate.

B35. The method of embodiment B34, wherein the lignocellulosic material comprises a plant or wood material.

B36. The method of embodiment B34 or B35, wherein the lignocellulosic substrate is an agricultural waste product.

B37. The method of embodiment B36, wherein the agricultural waste product is selected from the group consisting of corn stover, kenaf pith, canola straw and wheat straw.

B38. The method of embodiment B35, wherein the plant or wood material is purposefully harvested for use in the production of a mycelium.

B39. The method of embodiment B34 or B35, wherein the lignocellulosic material is not an agricultural waste product.

B40. The method of any one of embodiments B34 to B40, wherein the lignocellulosic material comprises hemp, maple, oak, oak pellets, corn, kenaf, canola, soy straw, soy flour, soybean hull pellets, wheat straw, seed or seed husk material; or a combination thereof.

B41. The method of embodiment B39 or B40, wherein the lignocellulosic material is not corn stover.

B42. The method of embodiment B40, wherein the seed is selected from the group consisting of sunflower seed, walnut and poppy seed; and combinations thereof.

B43. The method of embodiment B35, wherein the lignocellulosic material is a wood material, and wherein the wood material comprises a hardwood or a softwood material.

B44. The method of embodiment B43, wherein the hardwood or the softwood is of the genus Acer, Quercus, Populus, Abies or Pinus.

B45. The method of embodiment B35, B43 or B44, wherein the lignocellulosic material comprises wood flour, plant flour, wood chips, wood flakes, wood shavings, wood pellets or plant shavings.

B46. The method of embodiment B45, wherein the wood flour is maple wood flour.

B47. The method of embodiment B45, wherein the wood chips are maple wood chips, the wood flakes are maple wood flakes, and the wood shavings are maple wood shavings.

B48. The method of embodiment B45, wherein the plant flour is soy flour.

B49. The method of embodiment B33, wherein the natural substrate comprises a cellulosic material.

B50. The method of embodiment B49, wherein the cellulosic material is a lignin-free material.

B51. The method of embodiment B49 or B50, wherein the cellulosic material comprises plant fiber.

B52. The method of embodiment B51, wherein the plant fiber is a fiber obtained from cotton (Gossypium sp.), hemp (Cannabis sp.), flax (Linum sp.) or jute (Corchorus sp.).

B53. The method of embodiment B49, B50, B51 or B52, wherein the cellulosic material comprises pet bedding, paper, cardboard, card stock, cotton, linen or textile; or a combination thereof.

B54. The method of embodiment B33, wherein the natural substrate comprises an inorganic material; optionally, the natural substrate consists essentially of an inorganic material, or consists of an inorganic material.

B55. The method of embodiment B54, wherein the inorganic material is a mineral or mineral-based material.

B56. The method of embodiment B55, wherein the mineral or mineral-based material selected from the group consisting of vermiculite, perlite, soil, chalk, gypsum, clay, sand, rockwool and growstones; and combinations thereof.

B57. The method of embodiment B56, wherein the clay is expanded clay or clay in the form of beads.

B58. The method of embodiment B55, wherein the mineral or mineral-based material is a lignin-free material.

B59. The method of embodiment B32, wherein the substrate comprises a synthetic material.

B60. The method of embodiment B59, wherein the synthetic material is a plastic.

B61. The method of embodiment B59, wherein the synthetic material is a synthetic polymer.

B62. The method of embodiment B61, wherein the synthetic polymer is a synthetic organic polymer.

B63. The method of embodiment B62, wherein the synthetic organic polymer is selected from the group consisting of a polyethylene, a polypropylene, a polyvinyl chloride, a polystyrene, a polyacrylate, a nylon, a polytetrafluoroethylene (e.g., Teflon™), a polyamide, a polyester, a polysulfide, a polycarbonate, a polythene or a polyurethane.

B64. The method of embodiment B62 or B63, wherein the synthetic organic polymer contains one or more heteroatoms.

B65. The method of embodiment B64, wherein the synthetic organic polymer containing one or more heteroatoms is selected from the group consisting of a polyamide, a polyester, a polyurethane, a polysulfide and a polycarbonate; optionally, the synthetic organic polymer is a polyurethane, which is optionally a thermoplastic polyurethane.

B66. The method of any one of embodiments B59 to B65, wherein the synthetic material is obtained from a recycled material.

B67. The method of embodiment B32, wherein the substrate comprises an artificial material.

B68. The method of embodiment B67, wherein the artificial material comprises alginate, rayon, agar or agar-agar; optionally the rayon is rayon fiber, such as viscose.

B69. The method of embodiment B68, wherein the alginate is sodium alginate.

B71. The method of any one of embodiments B1 to B69, wherein the substrate is provided as a particles, said particles characterized as having a particle size.

B72. The method of embodiment B71, wherein the particle size is at most about 0.25 inch in diameter.

B73. The method of embodiment B72, wherein the particle size is less than 0.25 inch in diameter.

B74. The method of embodiment B71, wherein the particle size is at most about 0.125 inch in diameter.

B75. The method of embodiment B71, wherein the particle size is less than about 0.125 inch in diameter.

B76. The method of embodiment B71, wherein the particle size is at most about 0.01 inches in diameter.

B77. The method of embodiment B71, wherein the particle size is less than about 0.01 inch in diameter; optionally, at most about 0.007 inch in diameter.

B78. The method of embodiment B71, wherein the particle size is at least about 0.25 inch, or is greater than 0.25 inch in diameter.

B79. The method of embodiment B78, wherein the particle size is at most about 2 inches in diameter.

B80. The method of any one of embodiments B1 to B79, wherein the method further comprises sizing the substrate to a predetermined particle size prior to providing the growth matrix.

B81. The method of any one of embodiments B1 to B44 and B49 to B69, wherein the substrate is a monolithic substrate.

B82. The method of embodiment B81, wherein the monolithic substrate is a contiguous porous solid.

B83. The method of embodiment B82, wherein the monolithic substrate is a log, a slab of wood, textile or a solidified porous gel medium; or a combination thereof.

B84. The method of embodiment B82, wherein the monolithic substrate is a contiguous woven textile or a contiguous non-woven textile.

B85. The method of embodiment B84, wherein the contiguous woven or non-woven textile comprises rockwool, cotton (including nonwoven cotton), wood fiber or polyester fiber; optionally, the contiguous textile is provided in the form of a mat.

B86. The method of embodiment B82, wherein the monolithic substrate comprises a combination of two or more monolithic substrates.

B87. The method of any one of embodiments B1 to B86, wherein the substrate is a non-toxic substrate.

B88. The method of any one of embodiments B1 to B87, wherein the nutrient source is a lignocellulosic material.

B89. The method of embodiment B88, wherein the lignocellulosic material comprises seed, seed husks or both.

B90. The method of embodiment B89, wherein the seed is sunflower seed, walnut, or poppy seed; or a combination thereof.

B91. The method of any one of embodiments B1 to B90, wherein providing the growth matrix further comprises inoculating the substrate with the fungal inoculum.

B92. The method of any one of embodiments B1 to B91, wherein providing the growth matrix comprises inoculating a blend containing the substrate and the nutrient source with the fungal inoculum, thereby providing the growth matrix.

B93. The method of any one of embodiments B1 to B92, wherein the fungal inoculum is a seed-supported fungal inoculum, a grain-supported fungal inoculum, a seed-saw dust mixture fungal inoculum, or another commercially available fungal inoculum (for example, a specialty proprietary spawn type provided by inoculum retailers).

B94. The method of embodiment B93, wherein the fungal inoculum has a density of about 0.1 gram per cubic inch to about 10 grams per cubic inch, or from about 1 gram per cubic inch to about 7 grams per cubic inch; optionally, the fungal inoculum is a seed-supported or a grain-supported fungal inoculum.

B95. An edible aerial mycelium, prepared by the method of any of embodiments B1 to B94, wherein the edible aerial mycelium exhibits at least one of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

B96. An edible aerial mycelium, prepared by the process of any of embodiments B1 to B94, wherein the edible aerial mycelium exhibits at least two of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

Some other nonlimiting embodiments of the present disclosure are listed below.

C1. A foodstuff comprising an aerial mycelium, wherein the aerial mycelium exhibits at least one of the following physical characteristics: a mean thickness of at least about 10 mm, a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C2. A foodstuff comprising an edible aerial mycelium, wherein the edible aerial mycelium exhibits at least two of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C3. A foodstuff comprising an edible aerial mycelium, wherein the edible aerial mycelium exhibits at least three of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C4. A foodstuff comprising an edible aerial mycelium, wherein the edible aerial mycelium exhibits at least four of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C5. An edible aerial mycelium having at least one of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C6. An edible aerial mycelium having at least two of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C7. An edible aerial mycelium having at least three of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C8. An edible aerial mycelium having at least four of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C9. A manufactured edible aerial mycelium having at least one of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C10. A manufactured edible aerial mycelium having at least two of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C11. A manufactured edible aerial mycelium having at least three of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C12. A manufactured edible aerial mycelium having at least four of the following physical characteristics: a mean thickness of at least about 10 mm; a moisture content of at least about 80% (w/w); a mean native density within a range of about 1.8 to about 42 pounds per cubic foot (pcf); a Kramer shear force of no greater than about 15 kg/g; and a mean hyphal width of at most about 20 microns, at most about 15 microns, or within a range of about 0.2 to about 15 microns.

C13. A method of using an edible aerial mycelium to form a foodstuff, comprising combining the aerial mycelium with at least one additive; thereby forming a foodstuff.

C14. The method of embodiment C13, wherein the additive is a fat, a protein, an amino acid, a flavorant, an aromatic agent, a mineral, a vitamin, a micronutrient, a colorant or a preservative; or a combination thereof.

C15. The method of embodiment C13 or C15, wherein the foodstuff is a mycelium-based bacon product.

Some other nonlimiting embodiments of the present disclosure are listed below.

D1. An edible mycelium-based product comprising an edible aerial mycelium, wherein the edible aerial mycelium has at least two of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus at 10% strain of no greater than about 10 psi; and
ix. a native thickness of at least about 20 mm over at least about 80% of the aerial mycelium;
   wherein the edible aerial mycelium does not contain a fruiting body.

D2. An edible mycelium-based product comprising an edible aerial mycelium, wherein the edible aerial mycelium has at least three of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus at 10% strain of no greater than about 10 psi; and
ix. a native thickness of at least about 20 mm over at least about 80% of the aerial mycelium;
   wherein the edible aerial mycelium does not contain a fruiting body.

D3. An edible mycelium-based product comprising an edible aerial mycelium, wherein the edible aerial mycelium has at least four of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus at 10% strain of no greater than about 10 psi; and
ix. a native thickness of at least about 20 mm over at least about 80% of the aerial mycelium;
   wherein the edible aerial mycelium does not contain a fruiting body.

D4. An edible mycelium-based product comprising an edible aerial mycelium, wherein the edible aerial mycelium has at least five, at least six, at least seven, at least eight, or has each and every one of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus at 10% strain of no greater than about 10 psi; and
ix. a native thickness of at least about 20 mm over at least about 80% of the aerial mycelium;
   wherein the edible aerial mycelium does not contain a fruiting body.

D5. The edible mycelium-based product of any one of embodiments D1 to D4, wherein the edible aerial mycelial mean native density is within a range of about 1 pcf to about 15 pcf.

D6. The edible mycelium-based product of any one of embodiments D1 to D4, wherein the edible aerial mycelial mean native density is within a range of about 1 pcf to about 10 pcf.

D7. The edible mycelium-based product of embodiment D5 or D6, wherein the edible aerial mycelial mean native density is at least about 2 pcf, or is at least about 3 pcf.

D8. The edible mycelium-based product of any one of embodiments D1 to D4, wherein the edible aerial mycelial mean native density is within a range of about 3 pcf to about 6 pcf.

D9. The edible mycelium-based product of any one of embodiments D1 to D8, wherein the edible aerial mycelial native thickness is at least about 20 mm over at least about 90% of the aerial mycelium.

D10. The edible mycelium-based product of any one of embodiments D1 to D8, wherein the edible aerial mycelial native thickness is at least about 30 mm over at least about 80% of the aerial mycelium.

D11. The edible mycelium-based product of any one of embodiments D1 to D8, wherein the edible aerial mycelial native thickness is at least about 30 mm over at least about 90% of the aerial mycelium.

D12. The edible mycelium-based product of any one of embodiments D1 to D11, wherein the edible aerial mycelial native moisture content is at least about 90% (w/w).

D13. The edible mycelium-based product of any one of embodiments D1 to D12, wherein the ratio of the native ultimate tensile strength in the dimension substantially parallel to the direction of aerial mycelial growth, to the native ultimate tensile strength in the dimension substantially perpendicular to the direction of aerial mycelial growth, is about 3:1.

D14. The edible mycelium-based product of any one of embodiments D1 to D13, wherein the edible aerial mycelium exhibits a Kramer shear force in a dimension substantially parallel to the direction of aerial mycelial growth within a range of about 50 kg/g to about 120 kg/g after oven drying the edible aerial mycelium.

D15. The edible mycelium-based product of any one of embodiments D1 to D14, wherein the edible aerial mycelial native compressive modulus is within a range of about 0.58 psi to about 0.62 psi.

D16. The edible mycelium-based product of any one of embodiments D1 to D15, wherein the edible aerial mycelium has a native compressive stress at 10% compression within a range of about 0.05 psi to about 0.15 psi, or within a range of about 0.08 psi to about 0.13 psi.

D17. The edible mycelium-based product of any one of embodiments D1 to D16, wherein the edible aerial mycelium has a native protein content within a range of about 20% to about 50% (w/w), about 21% to about 49% (w/w), about 22% to about 48% (w/w), about 23% to about 47%, about 24% to about 46% (w/w), about 25% to about 45% (w/w), about 26% to about 44% (w/w), about 27% to about 43% (w/w) or about 28% to about 42% (w/w), on a dry weight basis.

D18. The edible mycelium-based product of any one of embodiments D1 to D17, wherein the edible aerial mycelium has a native potassium content of at least about 4000 mg per 100 grams of dry aerial mycelium.

D19. The edible mycelium-based product of embodiment D18, wherein the native potassium content is within a range of about 4000 mg to about 7000 mg potassium per 100 g dry aerial mycelium.

D20. The edible mycelium-based product of any one of embodiments D1 to D19, wherein the edible aerial mycelium has a native fat content of at most about 7% (w/w), or at most about 6% (w/w), on a dry weight basis.

D21. The edible mycelium-based product of any one of embodiments D1 to D20, wherein the edible aerial mycelium has a native carbohydrate content within a range of about 30% (w/w) to about 60% (w/w), about 35% (w/w) to about 55% (w/w), about 40% (w/w) to about 55% (w/w), about 40% (w/w) to about 50% (w/w), or about 45% (w/w) to about 55% (w/w), on a dry weight basis.

D22. The edible mycelium-based product of any one of embodiments D1 to D21, wherein the edible aerial mycelium has a native inorganic content within a range of about 5% (w/w) to about 20% (w/w), about 6% (w/w) to about 20% (w/w), about 7% (w/w) to about 20% (w/w), about 8% (w/w) to about 20% (w/w), about 9% (w/w) to about 20% (w/w), about 10% (w/w) to about 20% (w/w), or about 90% (w/w) to about 18% (w/w), on a dry weight basis.

D23. The edible mycelium-based product of any one of embodiments D1 to D22, wherein the edible aerial mycelium has a native dietary fiber content within a range of about 15% (w/w) to about 35% (w/w), on a dry weight basis.

D24. The edible mycelium-based product of any one of embodiments D1 to D23, wherein the edible aerial mycelium is white to off-white in color.

D25. The edible mycelium-based product of any one of embodiments D1 to D24, wherein the edible aerial mycelium is a growth product of a fungal species of the genus Pleurotus.

D26. The edible mycelium-based product of embodiment D25, wherein the fungal species is Pleurotus ostreatus.

D27. The edible mycelium-based product of any one of embodiments D1 to D26, wherein the edible mycelium-based product consists of the edible aerial mycelium.

D28. The edible mycelium-based product of any one of embodiments D1 to D26, wherein the edible aerial mycelium is a food ingredient suitable for use in the manufacture of an edible mycelium-based meat alternative product.

D29. The edible mycelium-based product of any one of embodiments D1 to D26, wherein the edible aerial mycelium is a food ingredient for use in the manufacture of an edible mycelium-based meat alternative product.

D30. The edible mycelium-based product of any one of embodiments D1 to D26, wherein the edible aerial mycelium is a food ingredient suitable for use in the manufacture of an edible mycelium-based bacon product.

D31. The edible mycelium-based product of any one of embodiments D1 to D26, wherein the edible aerial mycelium is a food ingredient for use in the manufacture of an edible mycelium-based bacon product.

Some other nonlimiting embodiments of the present disclosure are listed below.

E1. A batch of edible aerial mycelial panels, wherein greater than 50% of the edible aerial mycelial panels in the batch have at least two of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus within a range of about 0.5 psi to about 0.7 psi; and
ix. a native thickness of at least about 20 mm over at least about 80% of the aerial mycelium;
   wherein the edible aerial mycelium does not contain a fruiting body.

E2. A batch of edible aerial mycelial panels, wherein greater than 50% of the edible aerial mycelial panels in the batch have at least three of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus within a range of about 0.5 psi to about 0.7 psi; and
ix. a native thickness of at least about 20 mm over at least about 80% of the aerial mycelium;
   wherein the edible aerial mycelium does not contain a fruiting body.

E3. A batch of edible aerial mycelial panels, wherein greater than 50% of the edible aerial mycelial panels in the batch have at least four of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus within a range of about 0.5 psi to about 0.7 psi; and
ix. a native thickness of at least about 20 mm over at least about 80% of the aerial mycelium;
   wherein the edible aerial mycelium does not contain a fruiting body.

E4. A batch of edible aerial mycelial panels, wherein greater than 50% of the edible aerial mycelial panels in the batch have at least five of the following properties:
i. a mean native density of at least about 1 pcf;
ii. a native moisture content of at least about 80% (w/w);
iii. a native Kramer shear force in a dimension substantially parallel to the direction of aerial mycelial growth within a range of about 1.5 kilogram per gram (kg/g) of aerial mycelium to about 5.5 kg/g of aerial mycelium;
iv. a native Kramer shear force in a dimension substantially perpendicular to the direction of aerial mycelial growth within a range of about 2.5 kg/g to about 9 kg/g of aerial mycelium;
v. a native ultimate tensile strength in a dimension substantially parallel to the direction of aerial mycelial growth within a range of about 0.5 psi to about 1.6 psi;
vi. a native ultimate tensile strength in a dimension substantially perpendicular to the direction of aerial mycelial growth within a range of about 0.3 psi to about 0.5 psi;
vii. a native ultimate tensile strength in a dimension substantially parallel to the direction of aerial mycelial growth, and a native ultimate tensile strength in a dimension substantially perpendicular to the direction of aerial mycelial growth, in a ratio of about 2:1, about 2.5:1, about 3:1, about 3.5:1 or about 4:1;
viii. a native compressive modulus within a range of about 0.5 psi to about 0.7 psi; and
ix. a native thickness of at least about 20 mm over at least about 80% of the aerial mycelium;
wherein the edible aerial mycelium does not contain a fruiting body.

E5. The batch of edible aerial mycelial panels of any one of embodiments E1 to E4, wherein the edible aerial mycelial panel mean native density is within a range of about 1 pcf to about 15 pcf.

E6. The batch of edible aerial mycelial panels of any one of embodiments E1 to E4, wherein the edible aerial mycelial panel mean native density is within a range of about 1 pcf to about 10 pcf.

E7. The batch of edible aerial mycelial panels of embodiment E5, wherein the edible aerial mycelial panel mean native density is at least about 2 pcf, or is at least about 3 pcf.

E8. The batch of edible aerial mycelial panels of any one of embodiments E1 to E4, wherein the edible aerial mycelial panel mean native density is within a range of about 3 pcf to about 6 pcf.

E9. The batch of edible aerial mycelial panels of any one of embodiments E1 to E8, wherein the edible aerial mycelial panel native thickness is at least about 20 mm over at least about 90% of the aerial mycelium.

E10. The batch of edible aerial mycelial panels of any one of embodiments E1 to E8, wherein the edible aerial mycelial panel native thickness is at least about 30 mm over at least about 80% of the aerial mycelium.

E11. The batch of edible aerial mycelial panels of any one of embodiments E1 to E8, wherein the edible aerial mycelial panel native thickness is at least about 30 mm over at least about 90% of the aerial mycelium.

E12. The batch of edible aerial mycelial panels of any one of embodiments E1 to E11, wherein the edible aerial mycelial panel native moisture content is at least about 90% (w/w).

E13. The batch of edible aerial mycelial panels of any one of embodiments E1 to E12, wherein the ratio of the native ultimate tensile strength in the dimension substantially parallel to the direction of aerial mycelial growth, to the native ultimate tensile strength in the dimension substantially perpendicular to the direction of aerial mycelial growth, is about 3:1.

E14. The batch of edible aerial mycelial panels of any one of embodiments E1 to E13, wherein the edible aerial mycelial panel has the following additional property; a Kramer shear force in a dimension substantially parallel to the direction of aerial mycelial growth within a range of about 50 kg/g to about 120 kg/g after oven drying the edible aerial mycelial panel.

E15. The batch of edible aerial mycelial panels of any one of embodiments E1 to E14, wherein the edible aerial mycelial panel native compressive modulus is within a range of about 0.58 psi to about 0.62 psi.

E16. The batch of edible aerial mycelial panels of any one of embodiments E1 to E15, wherein the edible aerial mycelial panel has the following additional property: a native compressive stress at 10% compression within a range of about 0.05 psi to about 0.15 psi, or within a range of about 0.08 psi to about 0.13 psi.

E17. The batch of edible aerial mycelial panels of any one of embodiments E1 to E16, wherein the edible aerial mycelial panel has the following additional property: a native protein content within a range of about 20% to about 50% (w/w), about 21% to about 49% (w/w), about 22% to about 48% (w/w), about 23% to about 47%, about 24% to about 46% (w/w), about 25% to about 45% (w/w), about 26% to about 44% (w/w), about 27% to about 43% (w/w) or about 28% to about 42% (w/w), on a dry weight basis.

E18. The batch of edible aerial mycelial panels of any one of embodiments E1 to E17, wherein the edible aerial mycelial panel has the following additional property: a native potassium content of at least about 4000 mg per 100 grams of dry aerial mycelium.

E19. The batch of edible aerial mycelial panels of embodiment E18, wherein the native potassium content is within a range of about 4000 mg to about 7000 mg potassium per 100 g dry aerial mycelium.

E20. The batch of edible aerial mycelial panels of any one of embodiments E1 to E19, wherein the edible aerial mycelial panel has the following additional property: a native fat content of at most about 7% (w/w), or at most about 6% (w/w), on a dry weight basis.

E21. The batch of edible aerial mycelial panels of any one of embodiments E1 to E20, wherein the edible aerial mycelial panel has the following additional property: a native carbohydrate content within a range of about 30% (w/w) to about 60% (w/w), about 35% (w/w) to about 55% (w/w), about 40% (w/w) to about 55% (w/w), about 40% (w/w) to about 50% (w/w), or about 45% (w/w) to about 55% (w/w), on a dry weight basis.

E22. The batch of edible aerial mycelial panels of any one of embodiments E1 to E21, wherein the edible aerial mycelial panel has the following additional property: a native inorganic content within a range of about 5% (w/w) to about 20% (w/w), about 6% (w/w) to about 20% (w/w), about 7% (w/w) to about 20% (w/w), about 8% (w/w) to about 20% (w/w), about 9% (w/w) to about 20% (w/w), about 10% (w/w) to about 20% (w/w), or about 9% (w/w) to about 18% (w/w), on a dry weight basis.

E23. The batch of edible aerial mycelial panels of any one of embodiments E1 to E22, wherein the edible aerial mycelial panel has the following additional property: a native dietary fiber content within a range of about 15% (w/w) to about 35% (w/w), on a dry weight basis.

E24. The batch of edible aerial mycelial panels of any one of embodiments E1 to E23, wherein the edible aerial mycelial panel has the following additional property: being white to off-white in color.

E25. The batch of edible aerial mycelial panels of any one of embodiments E1 to E24, wherein each edible aerial mycelial panel in the batch is a growth product of a fungal species of the genus Pleurotus.

E26. The batch of edible aerial mycelial panels of embodiment E25, wherein the fungal species is Pleurotus ostreatus.

E27. The batch of edible aerial mycelial panels of any one of embodiments E1 to E26, wherein at least 75% of the edible aerial mycelial panels in the batch have at least two, at least three, at least four or at least five of said properties.

E28. The batch of edible aerial mycelial panels of any one of embodiments E1 to E27, wherein the edible aerial mycelial panel is a food ingredient suitable for use in the manufacture of an edible mycelium-based meat alternative product.

E29. The batch of edible aerial mycelial panels of any one of embodiments E1 to E27, wherein the edible aerial mycelial panel is a food ingredient for use in the manufacture of an edible mycelium-based meat alternative product.

E30. The batch of edible aerial mycelial panels of any one of embodiments E1 to E27, wherein the edible aerial mycelial panel is a food ingredient suitable for use in the manufacture of an edible mycelium-based bacon product.

E31. The batch of edible aerial mycelial panels of any one of embodiments E1 to E27, wherein the edible aerial mycelial panel is a food ingredient for use in the manufacture of an edible mycelium-based bacon product.

E32. The batch of edible aerial mycelial panels of any one of embodiments E1 to E31, wherein the batch is a quantity of at least ten edible aerial mycelial panels.

E33. The batch of edible aerial mycelial panels of any one of embodiments E1 to E32, wherein the batch quantity is at most about 100 edible aerial mycelial panels.

Some other nonlimiting embodiments of the present disclosure are listed below.

F1. A method of processing an edible aerial mycelium, comprising:
(a) providing a panel comprising an edible aerial mycelium, wherein the edible aerial mycelium is characterized as having a direction of mycelial growth along a first axis;
(b) performing a physical method comprising:
   compressing the panel in a compressing direction which is substantially non-parallel with respect to the first axis to form a compressed panel;
   optionally, sectioning the compressed panel to form at least one compressed section;
   cutting the compressed panel, or optionally the at least one compressed section, in a cutting direction which is substantially parallel to the first axis to form at least one compressed strip; and
   optionally, perforating the at least one compressed strip to form at least one perforated strip;
(c) boiling the at least one compressed strip, or optionally the at least one perforated strip, in a first aqueous saline solution to form at least one boiled strip;
(d) brining the at least one boiled strip to provide at least one brined strip;
(e) drying the at least one brined strip to provide at least one dried strip; and
(f) adding fat to the at least one dried strip to provide at least one fattened strip.

F2. The method of F1, wherein the compressing comprises compressing the panel to about 15% to about 75% of the original panel length or width.

F3. The method of F2, wherein the compressing comprises compressing the panel to about 30% to about 40% of the original panel length or width.

F4. The method of any one of F1 to F3, wherein the compressing direction is within a range of greater than 45 degrees and less than 135 degrees, or greater than about 70 degrees and less than about 110 degrees, with respect to the first axis.

F5. The method of any one of F1 to F3, wherein the compressing direction is substantially orthogonal to the first axis.

F6. The method of any one of F1 to F5, wherein the cutting direction is within a range of plus or minus about 45 degrees with respect to the first axis, or is within a range of plus or minus about 30 degrees with respect to the first axis.

F7. The method of any one of F1 to F6, wherein the method further comprises sectioning the compressed panel to form at least one compressed section.

F8. The method of F7, wherein the sectioning comprises cutting the panel in the cutting direction to form the at least one compressed section.

F9. The method of any of F1 to F8, wherein the physical method comprises perforating the at least one compressed strip to form the at least one perforated strip.

F10. The method of F9, wherein the perforating comprises needling.

F11. The method of F10, wherein needling comprise inserting at least one needle into the outer surface of the at least one compressed strip.

F12. The method of F11, wherein the at least one needle is straight or barbed.

F13. The method of F10, F11 or F12, wherein the needling comprises inserting the at least one needle through entirely through the mycelial tissue of the at least one compressed strip.

F14. The method of any one of F1 to F13, wherein perforating the at least one compressed strip comprises a first perforation step forming a first perforation pattern, and a second perforation step forming a second perforation pattern.

F15. The method of F14, wherein at least one of the density, intensity and shape of the first perforation pattern is different from the density, intensity and shape of the second perforation pattern.

F16. The method of any one of F9 to F15, wherein the at least one edible strip comprises a plurality of strips stacked relative to each other.

F17. The method of any one of F1 to F16, wherein the first aqueous saline solution has a salt concentration within a range of about 0.1% (w/w) to about 26% (w/w), about 0.1% to about 15% (w/w), about 0.5% to about 10% (w/w), about 0.5% to about 5% (w/w) or about 1% to about 3%.

F18. The method of any one of F1 to F17, wherein the first aqueous saline solution further comprises at least one an additive.

F19. The method of any one of F1 to F18, wherein the brining comprises treating the at least one boiled strip with a brine fluid to provide the at least one brined strip.

F20. The method of F19, wherein the brine fluid is a second aqueous saline solution having a salt concentration within a range of about 0.1% (w/w) to about 26% (w/w), about 0.1% to about 15% (w/w), about 0.5% to about 10% (w/w), about 0.5% to about 5% (w/w) or about 1% to about 3%.

F21. The method of F19 or F20, wherein the brine fluid further comprises at least one additive.

F22. The method of F21, wherein the at least one additive is a flavorant, a colorant, or both.

F23. The method of any one of F19 to F22, wherein the brine fluid comprises a smoke flavorant, umami, maple, a salt, a sweetener, a spice, or a combination of any two or more of the foregoing.

F24. The method of any one of F19 to F23, wherein the brining comprises submerging the at least one boiled strip in the brine fluid.

F25. The method of any one of F19 to F24, wherein the brining further comprises simmering the at least one boiled strip in the brine fluid.

F26. The method of any one of F19 to F25, further comprising removing the at least one brined strip from the brine fluid.

F27. The method of F1 to F26, wherein the drying comprises heating the at least one brined strip.

F28. The method of any one of F1 to F27, wherein the method further comprises cooling the at least one fattened strip.

F29. The method of F28, wherein the cooling comprises cooling the at least one fattened strip until the fat is solidified.

F30. The method of F28 or F29, wherein the cooling comprises refrigerating the at least one fattened strip.

F31. The method of F28, F29 or F30, wherein the method provides at least one finished edible strip.

F32. The method of F1 to F31, further comprising packaging the at least one strip.

F33. The method of F1 to F32, wherein each said at least one strip is a plurality of strips.

F34. The method of F1 to F33, wherein the panel is characterized as having a mean native thickness of at least about 20 mm, at least about 30 mm, at least about 40 mm or at least about 50 mm.

F35. The method of any one of F1 to F34, wherein the edible aerial mycelium is the edible aerial mycelium of the present disclosure.

F36. The method of F1 to F35, wherein the panel consists essentially of the edible aerial mycelium.

F37. The method of F1 to F35, wherein the panel consists of the edible aerial mycelium.

F38. The method of F1 to F37, wherein the fat is almond oil, animal fat, avocado oil, butter, canola oil, coconut oil, corn oil, grapeseed oil, hempseed oil, lard, mustard oil, olive oil, palm oil, peanut oil, rice bran oil, safflower oil, soybean oil, sunflower seed oil, vegetable oil, vegetable shortening or animal fat; or a combination thereof.

F39. The method of F1 to F38, wherein the fat further comprises a colorant, flavorant, or both.

F40. The method of F39, wherein the flavorant is umami, maple, a salt, a sweetener, a spice, or a combination of any two or more of the foregoing.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from (i.e. plus or minus) exactly parallel by less than or equal to 45 degrees, 40 degrees, 35 degrees, 30 degrees, 25 degrees, 20 degrees, 15 degrees, 10 degrees, 5 degrees, 3 degrees, 1 degree, or 0.1 degree, and any ranges therebetween. As another example, in certain embodiments, the term “substantially non-parallel” refers to a value, amount, or characteristic that departs from (i.e. plus or minus) exactly zero or 180 degrees by more than 45 degrees, 50 degrees, 55 degrees, 60 degrees, 65 degrees, 70 degrees, 75 degrees, 80 degrees, 85 degrees, and up to 90 degrees, and any ranges therebetween. As another example, in certain embodiments, the terms “generally orthogonal,” “generally perpendicular,” “substantially orthogonal” and “substantially perpendicular” refer to a value, amount, or characteristic that departs from (i.e. plus or minus) exactly 90 degrees by less than or equal to 15 degrees, 10 degrees, 5 degrees, 3 degrees, 1 degree, or 0.1 degree, and any ranges therebetween.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1-212. (canceled)

213. A system for growing an edible aerial mycelium, comprising:
a growth matrix comprising a substrate and a fungal inoculum, wherein the fungal inoculum comprises a fungus;
a growth environment configured to incubate the growth matrix as a solid-state culture for an incubation time period;
a misting apparatus; and
an atmospheric control system with an electronic controller configured to maintain a carbon dioxide (CO₂) level within the growth environment between at least about 0.02% (v/v) and less than about 8% (v/v) and to introduce aqueous mist into the growth environment with the misting apparatus throughout the incubation time period, or a portion thereof, at a mist deposition rate and a mean mist deposition rate, wherein the mist deposition rate is less than or equal to about 150 microliter/cm²/hour, and the mean mist deposition rate is less than or equal to about 3 microliter/cm²/hour.

214. The system of claim 213, wherein the atmospheric control system is configured to maintain the growth environment at a relative humidity of at least about 95%.

215. The system of claim 213, wherein the atmospheric control system is configured to maintain the growth environment at a temperature within a range of 55° F. to 100° F.

216. The system of claim 213, wherein the atmospheric control system is configured to maintain the ratio of the mist deposition rate to the mean mist deposition rate within a range of about 100:1 to about 1000:1.

217. The system of claim 213, wherein the aqueous mist comprises one or more solutes.

218. The system of claim 213, wherein the misting apparatus is configured to deposit the aqueous mist onto the growth matrix, the extra-particle aerial mycelial growth, or both.

219. The system of claim 213, wherein the system is configured to provide a substantially horizontal airflow across the growth matrix.

220. The system of claim 219, wherein the substantially horizontal airflow has a velocity of no greater than about 275 linear feet per minute.

221. The system of claim 213, wherein the atmospheric control system is configured to maintain an O₂ level within the growth environment between a range of about 14% to about 21% (v/v).

222. The system of claim 213, wherein the CO₂ level is within a range of about 0.2% to about 7% (v/v).

223. The system of claim 213, wherein the fungus is a filamentous fungus.

224. The system of claim 213, wherein the growth environment is a dark environment.

225. The system of claim 213, wherein the substrate is a natural substrate.

226. The system of claim 225, wherein the natural substrate comprises a lignocellulosic material.

227. The system of claim 225, wherein the natural substrate comprises a cellulosic material.

228. The system of claim 227, wherein the cellulosic material is a lignin-free material.

229. The system of claim 225, wherein the natural substrate comprises an inorganic material.

230. The system of claim 229, wherein the inorganic material is comprises a mineral-based material.

231. The system of claim 213, wherein the fungal inoculum produces mycelium.

232. The system of claim 231, wherein the mycelium does not include any visible stipe, cap or spores.

233. The system of claim 231, wherein the mycelium grows vertically to produce an aerial mycelium.

234. The system of claim 233, wherein the aerial mycelium has a mean density of at least 1 pound per cubic food (pcf).

235. The system of claim 233, wherein the aerial mycelium has a mean thickness of at least 15 millimeters.
